(19) United States
(12) Patent Application Publication
(10) Pub. No.: US 2011/0195847 A1
(43) Pub. Date: Aug. 11, 2011

(54) METHODS TO TREAT SOLID TUMORS

(76) Inventors: Nabil Arrach, Newport Beach (CA); Michael McClelland, Carlsbad (CA)

(21) Appl. No.: 12/996,754

(22) PCT Filed: Jun. 12, 2009
(86) PCT No.: PCT/US09/47285

§ 371 Date: Apr. 28, 2011

Related U.S. Application Data
(60) Provisional application No. 61/061,576, filed on Jun. 13, 2008.

(51) Int. Cl. C40B 20/00; C12N 15/63; C12N 1/21

(52) U.S. Cl. 506/2; 435/320.1; 435/252.3

(57) ABSTRACT

A high throughput method for identifying promoters differentially activated in solid tumors as compared to normal tissues is described. The promoters so identified may be used to drive production of RNA's or proteins useful in treating solid tumors including toxic RNA's or proteins and other therapeutic RNA's or proteins.

### RELATED PATENT APPLICATION(S)

This application is a national stage of international patent application number PCT/US2009/047285, filed on Jun. 12, 2009, entitled “Methods to Treat Solid Tumors”, naming Nabil Arrach and Michael McClelland as inventors, and designated by attorney docket no. VIV-1001-PC, which claims the benefit of U.S. provisional patent application No. 61/061,576 filed on Jun. 13, 2008, entitled “Method to Treat Solid Tumors, and designated by Attorney Docket number 655233000100. The entire content of the foregoing patent applications is incorporated herein by reference, including, without limitation, all text, tables and drawings.

### STATEMENT OF GOVERNMENT SUPPORT

This invention was made in part with government support under Grant Nos. R01 AI034829, R01 AI052237, and R21 AI057733 awarded by the National Institutes of Health (NIH) and Grant Nos. TRDRP 16KT-0045 to Sidney Kimmel Cancer Center from the Tobacco-Related Disease Research Program of California and grants CA 103563; CA 119811 and DCD grant W81XWH-06-0117 to AntiCancer. The government has certain rights in this invention.

### FIELD OF THE INVENTION

The invention relates in part to compositions and methods selectively to target solid tumors. More specifically, it concerns compositions comprising expression systems for cytotoxic proteins under the control of promoters active in tumors.

### BACKGROUND

A wide range of bacteria (e.g., Escherichia, Salmonella, Clostridium, Listeria, and Bifidobacterium, for example) have been shown to preferentially colonize solid tumors. Salmonella enterica and avirulent derivatives may effect some degree of tumor reduction by the presence of the bacteria in the solid tumor. The internal environment of solid tumors is not well understood and may present favorable growing conditions to colonizing bacteria.

### SUMMARY

The environment inside solid tumors is very different from that in normal, healthy tissue. Solid tumors often are poorly vascularized and sometimes have areas of necrosis. The poor vascularization contributes to hypoxic or anoxic areas that can extend to about 100 micrometers from the vasculature of the solid tumor. Solid tumors also can have an internal pH lower than the organism's normal pH. Necrosis in solid tumors can lead to a nutrient rich environment where bacteria capable of growing in low oxygen conditions can flourish. In addition to the nutrient rich environment, the internal spaces of solid tumors also offer some degree of protection from a host organisms' immune system, and thus shield the bacteria from the hosts' immune response. These conditions may cause bacteria to express genes that are not normally expressed in normal, healthy tissues. These factors may contribute to the preferential colonization of solid tumors as compared to other normal tissue.

The internal environment of tumors may offer regulatory conditions not well understood, in addition to low oxygen and low pH. Promoters are nucleotide sequences that in part regulate the production of mRNA from coding sequences in genomic DNA. The mRNA then can be translated into a polypeptide having a particular biological activity. Bacterial promoters that are preferentially activated in tumors have been identified by methods described herein, and compositions that contain such promoters, and methods for using them, also are described.

Thus, provided herein are isolated nucleic acid molecules that comprise a recombinant expression system, which expression system comprises a nucleotide sequence encoding a toxic or therapeutic RNA (e.g., mRNA, tRNA, rRNA, siRNA, ribozyme, and the like), a protein or an RNA or protein that participates in generating a toxin or therapeutic agent, or a nucleotide sequence encoding a toxic or therapeutic agent, RNA or protein which can mobilize the subjects immune response, operably linked to a heterologous promoter which promoter is preferentially activated in solid tumors. In certain embodiments, the heterologous promoter sequence can be a naturally occurring promoter sequence. In some embodiments the promoter can be an Enterobacteriaceae promoter, and in certain embodiments the promoter is a Salmonella promoter. In some embodiments, the promoter may comprise (i) a nucleotide sequence of Table 2A, (ii) a functional promoter nucleotide sequence 80% or more identical to a nucleotide sequence of Table 2A, or (iii) or a functional promoter subsequence of (i) or (ii). In certain embodiments, the functional promoter subsequence is about 20 to about 150 nucleotides in length.

The term “preferentially activated in solid tumors” as used herein refers to a nucleotide sequence that expresses a polypeptide from a coding sequence in tumors at a level of at least two-fold more than the same polypeptide from the same coding sequence is expressed in non-tumor cells. The polypeptide may be expressed at detectable levels in non-tumor cells or tissue in some embodiments, and in certain embodiments, the polypeptide is not detectably expressed in non-tumor cells or tissue. As an example, preferential activation can be determined using (i) cells from the spleen as non-tumor cells and (ii) PC3 prostate cancer cells in a tumor xenograft for tumor cells. A reference level of the amount of polypeptide produced can be determined by the promoter expression in the bacterial culture samples, before injecting aliquots of the sample into mice (e.g., measuring GFP expression in the overnight cultures prepared to inject mice, also known as the input library). In some embodiments, preferential activation in solid tumors is identified by utilizing spleen, PC3 tumor xenograft and reference level (i.e., input) determinations described in Example 2 hereafter. In certain embodiments, a promoter is preferentially activated in a tumor of a living organism. In some embodiments, there can be two references used on the arrays described in Examples 1 and 2. One reference can be a library of all plasmids extracted from bacteria grown overnight in LB+ Amp (see below) culture broth, as described above. Another suitable reference that can be used would be to compare the profile of bacteria expressing GFP from a particular tissue of interest to the profile of all bacteria (e.g., GFP expresser and non-expressers, for example) isolated from the same tissue of interest.

Also provided are suitable delivery vectors for administering the isolated nucleic acid which may comprise a recombinant expression system. In some embodiments, recombinant host cells that contain the nucleic acid molecules described above or below may be used to delivery the expression system to a patient or subject. In certain embodiments, the cells may be avirulent Salmonella cells. Also provided are pharmaceutical compositions which can comprise the nucleic acid reagents isolated, generated or modified by methods described herein, or cells which harbor such nucleic acid reagents.

Also provided, in certain embodiments, are methods to treat solid tumors, which methods can comprise administering to a subject harboring a tumor the nucleic acid molecules isolated or generated as described herein, the cells containing them or compositions comprising the nucleic acid reagents and/or cells harboring them.

Also provided, in some embodiments, are methods for identifying a promoter preferentially activated in tumor tissue which method comprises: (a) providing a library of expression systems each may comprise a nucleotide sequence encoding a detectable protein operably linked to a different candidate promoter; (b) providing the library to solid tumor tissue and to normal tissue; (c) identifying cells from each tissue that show high levels of expression of the detectable protein; and (d) obtaining the expressions systems from the cells that produce greater levels of detectable protein in tumor tissue as compared to normal tissue, and identifying the promoters of the expression system. In some embodiments, the method may further comprise scoring the promoters identified in (d) (e.g., described below in Example 2). In some embodiments, the library is provided in recombinant host cells. In certain embodiments, the library of DNA fragments can be a random set of fragments from a bacterial genome (e.g., Salmonella genome, for example) in the range of about 25 to about 10,000 base pairs (bp) in length, for example. In some embodiments, the library may comprise known nucleic acid regions or known promoter regions from a bacterial genome in the range of about 25 to about 10,000 by in length, for example.

In certain embodiments, the promoters can be Salmonella promoters and the recombinant host cells can be Salmonella. In some embodiments, the candidate promoters are from bacteria, or are 80% or more identical to promoters from bacteria. In certain embodiments, the bacteria can be Enterobacteriaceae, and in some embodiments the Enterobacteriaceae can be Salmonella. Also provided, in some embodiments, is an expression system which comprises a nucleotide sequence encoding a toxic or therapeutic RNA or protein or an RNA or protein that participates in generating a desired toxin or therapeutic agent operably linked to a promoter identified by the methods described herein. Also provided herein, in certain embodiments, are recombinant host cells that may comprise an expression system described herein.

Also provided, in certain embodiments, are methods to treat solid tumors which methods comprise administering an expression system described herein or cells containing an expression system described herein, to a subject harboring a solid tumor.

Also provided, in some embodiments, is an expression system which may comprise a first promoter nucleotide sequence operably linked to a first coding sequence and second promoter nucleotide sequence operably linked to a second coding sequence, where: the first coding sequence and the second coding sequence encode polypeptides that individually do not inhibit tumor growth; polypeptides encoded by the first coding sequence and the second coding sequence, in combination, inhibit tumor growth; and the first promoter nucleotide sequence and the second promoter nucleotide sequence can be preferentially activated in solid tumors of living organisms. In certain embodiments, one or more of the promoter nucleotide sequences can be preferentially activated in solid tumors (e.g., one promoter is constitutive and one promoter is preferentially activated in solid tumors). In some embodiments, the first promoter nucleotide sequence and the second promoter nucleotide sequence can be in the same nucleic acid molecule. In certain embodiments, the first promoter nucleotide sequence and the second promoter nucleotide sequence may be in different nucleic acid molecules. In some embodiments, the first promoter nucleotide sequence and the second promoter nucleotide sequence can be bacterial nucleotide sequences. In certain embodiments, the bacterial sequences may be Enterobacteriaceae sequences, and in some embodiments the Enterobacteriaceae sequences can be Salmonella sequences. In certain embodiments, the different nucleic acid molecules can be disposed in the same recombinant host cell, and in some embodiments, the different nucleic acid molecules can be disposed in different recombinant host cells of the same species. In some embodiments, the different recombinant host cells can be different bacterial species.

In some embodiments, expression systems as described herein can produce two components that interact to provide a functional therapeutic agent, where: a first coding sequence may encode an enzyme, a second coding sequence may encode a prodrug, and the enzyme can process the prodrug into a drug that inhibits tumor growth. In certain embodiments, expression systems as described herein can produce two components that interact to provide a functional therapeutic agent, where; the first coding sequence may encode a first polypeptide, the second coding sequence can encode a second polypeptide, and the first polypeptide and the second polypeptide can form a complex that inhibits tumor growth.

In some embodiments, the first promoter nucleotide sequence, the second promoter nucleotide sequence, or the first promoter nucleotide sequence and the second promoter nucleotide sequence can comprise (i) a nucleotide sequence of Table 2A, (ii) a functional promoter nucleotide sequence 80% or more identical to a nucleotide sequence of Table 2A, or (iii) or a functional promoter subsequence of (i) or (ii). In certain embodiments, the functional promoter subsequence is about 20 to about 150 nucleotides in length. In some embodiments, expression systems described herein may be contained in recombinant host cells, and in certain embodiments, the recombinant host cells can be avirulent Salmonella.

Also provided, in certain embodiments, is an expression system which comprises three or more promoters operably linked to three or more coding sequences, where one, two, or more of the promoter nucleotide sequences are preferentially activated in solid tumors. In some embodiments, the coding sequences encode polypeptides that individually do not inhibit tumor growth and polypeptides encoded by the coding sequences, in combination, inhibit tumor growth.

Certain embodiments are described further in the following description, examples, claims and drawings.

### BRIEF DESCRIPTION OF THE DRAWINGS

The drawings illustrate embodiments of the invention and are not limiting. For clarity and ease of illustration, the drawings are not made to scale and, in some instances, various aspects may be shown exaggerated or enlarged to facilitate an understanding of particular embodiments.

FIG. 1 is a flow diagram illustrating the procedure used to construct the nucleic acid libraries used to identify and isolate Salmonella genomic sequences corresponding to promoter elements.

FIG. 2 shows photographs taken of tumors expressing GFP, demonstrating the in vivo function of the promoter elements identified and isolated using the methods described herein.

### DETAILED DESCRIPTION

Methods and compositions described herein have been designed to identify and isolate nucleic acid promoter sequences that can be preferentially activated under unique conditions found inside solid tumors of living organisms. Without being limited by any particular theory or to any particular class of inducible promoters, promoter identification methods described herein may be utilized to identify all classes of promoters that are preferentially active in solid tumors of living organisms. In some embodiments, promoter identification methods described herein can potentially identify promoters activated by the following classes of regulatory agents, including but not limited to, gases (e.g., oxygen, nitrogen, carbon dioxide and the like), pH (e.g., acidic pH or basic pH), metals (e.g., iron, copper and the like), hormones (e.g., steroids, peptides and the like), and various cellular components (e.g., purines, pyrimidines, sugars, and the like). The methods and compositions described herein also can be used to identify promoters preferentially active in any part of the body of a living organism, including wounds or diseased parts of the body, for example.

Non-limiting examples of solid tumors that may be treated by methods and compositions described herein are sarcomas (e.g., rhabdomyosarcoma, osteosarcoma, and the like, for example), lymphomas, blastomas (e.g., hepatocblastoma, retinoblastoma, and neuroblastom, for example), germ cell tumors (e.g., choriocarcinoma, and endodermal sinus tumor, for example), endocrine tumors, and carcinomas (e.g., adrenocortical carcinoma, colorectal carcinoma, hepatocellular carcinoma, for example).

Promoter elements preferentially activated in solid tumors of living organisms, identified and isolated using the methods described herein, can be used in targeted, tumor specific therapies. In some embodiments a promoter nucleotide sequence (e.g., heterologous promoter) is operably linked to a nucleotide sequence encoding one or more therapeutic agents. In some embodiments, the promoter sequence can be a naturally occurring nucleic acid sequence. A therapeutic agent includes, without limitation, a toxin (e.g., ricin, diphtheria toxin, abrin, and the like), a peptide, polypeptide or protein with therapeutic activity (e.g., methioninase, nitroreductase, antibody, antibody fragment, single chain antibody), a prodrug (e.g., CB1954), an RNA molecule (e.g., siRNA, ribozyme and the like, for example). The structures of such therapeutic agents are known and can be adapted to systems described herein, and can be from any suitable organism, such as a prokaryote (e.g., bacteria) or eukaryote (e.g., yeast, fungi, reptile, avian, mammal (e.g., human or non-human)), for example.

Antibodies sometimes are IgG, IgM, IgA, IgE, or an isotype thereof (e.g., IgG1, IgG2a, IgG2b or IgG3), sometimes are polyclonal or monoclonal, and sometimes are chimeric, humanized or bispecific versions of such antibodies. Polyclonal and monoclonal antibodies that bind specific antigens are commercially available, and methods for generating such antibodies are known. In general, polyclonal antibodies are produced by injecting an isolated antigen into a suitable animal (e.g., a goat or rabbit); collecting blood and/or other tissues from the animal containing antibodies specific for the antigen and purifying the antibody. Methods for generating monoclonal antibodies, in general, include injecting an animal with an isolated antigen (e.g., often a mouse or a rat); isolating splenocytes from the animal; fusing the splenocytes with myeloma cells to form hybridomas; isolating the hybridomas and selecting hybridomas that produce monoclonal antibodies which specifically bind the antigen (e.g., Kohler & Milstein, Nature 256:495 497 (1975) and StGroth & Scheidegger, J Immunol Methods 5:1 21 (1980)). Examples of monoclonal antibodies are anti MDM 2 antibodies, anti-p53 antibodies (pAB421, DO 1, and an antibody that binds phosphoryl-ser15), anti-dsDNA antibodies and anti-BrdU antibodies, are described hereafter.

Methods for generating chimeric and humanized antibodies also are known (see, e.g., U.S. Pat. No. 5,530,101 (Queen, et al.), U.S. Pat. No. 5,707,622 (Fung, et al.) and U.S. Pat. Nos. 5,994,524 and 6,245,894 (Matsushima, et al.)), which generally involve transplanting an antibody variable region from one species (e.g., mouse) into an antibody constant domain of another species (e.g., human). Antigen-binding regions of antibodies (e.g., Fab regions) include a light chain and a heavy chain, and the variable region is composed of regions from the light chain and the heavy chain. Given that the variable region of an antibody is formed from six complementarity-determining regions (CDRs) in the heavy and light chain variable regions, one or more CDRs from one antibody can be substituted (i.e., grafted) with a CDR of another antibody to generate chimeric antibodies. Also, humanized antibodies are generated by introducing amino acid substitutions that render the resulting antibody less immunogenic when administered to humans.

An antibody sometimes is an antibody fragment, such as a Fab, Fab′, F(ab)′2, Dab, Fv or single-chain Fv (ScFv) fragment, and methods for generating antibody fragments are known (see, e.g., U.S. Pat. Nos. 6,099,842 and 5,990,296 and PCT/GB00/04317). In some embodiments, a binding partner in one or more hybrids is a single-chain antibody fragment, which sometimes are constructed by joining a heavy chain variable region with a light chain variable region by a polypeptide linker (e.g., the linker is attached at the C-terminus or N-terminus of each chain) by recombinant molecular biology processes. Such fragments often exhibit specificities and affinities for an antigen similar to the original monoclonal antibodies. Bifunctional antibodies sometimes are constructed by engineering two different binding specificities into a single antibody chain and sometimes are constructed by joining two Fab′ regions together, where each Fab′ region is from a different antibody (e.g., U.S. Pat. No. 6,342,221). Antibody fragments often comprise engineered regions such as CDR-grafted or humanized fragments. In certain embodiments the binding partner is an intact immunoglobulin, and in other embodiments the binding partner is a Fab monomer or a Fab dimer.

In some embodiments, one or more promoter elements preferentially active in the solid tumors of living organisms may be operably linked, on the same or different nucleic acid reagents, to nucleotide sequences that can encode one or more components of a multi-component (e.g., two or more components) therapeutic agent. Therapeutic agents for such applications include, without limitation, an enzyme coding sequence, a prodrug coding sequence; a protein comprising two peptide sequences that interact to form the therapeutic agent; related genes from a metabolic pathway; or one or more RNA molecules that functionally interact to form a therapeutic agent, for example. In certain embodiments targeted, tumor specific therapies may comprise an expression system that may comprise a nucleic acid reagent contained in a recombinant host cell. The term “operably linked” as used herein refers to a nucleic acid sequence (e.g., a coding sequence) present on the same nucleic acid molecule as a promoter element and whose expression is under the control of said promoter element.

Expression Systems

Embodiments described herein provide an expression system useful for delivering a therapeutic agent or pharmaceutical composition (e.g., toxin, drug, prodrug, or microorganism (e.g. recombinant host cell) expressing a toxin, drug, or prodrug) to a specific target or tissue within a living subject exhibiting a condition treatable by the therapeutic agent or pharmaceutical composition (e.g., living organism with a solid tumor, for example). Embodiments described herein also may be useful for driving production of a system for generating toxic substances or to elicit responses from the host, for example by expressing cytokines, interleukins, growth inhibitors, or therapeutic RNA's or proteins from the expression system or causing the host organism to increase expression of cytokines, interleukins, growth inhibitors, or therapeutic RNA's or proteins by expression of an agent which can elicit the appropriate metabolic or immunological response. In some embodiments, the expression system may comprise a nucleic acid reagent and a delivery vector. The delivery vector sometimes can be a microorganism (e.g., bacteria, yeast, fungi, or virus) that harbors the nucleic acid reagent, and can express the product of the nucleic acid reagent or can deliver the nucleic acid reagent to the subject for expression within host cells.

In some embodiments, an expression system may comprise a promoter element operably linked to a therapeutic gene of a nucleic acid reagent. The nucleic acid reagent may be disposed in a bacterial host, where the bacterial host comprising the nucleic acid reagent is delivered to a eukaryotic organism such that expression of the nucleic acid reagent, in the appropriate tissue or structure (e.g., inside a solid tumor, for example) causes a therapeutic effect. In certain embodiments, the expression system promoter elements sometimes can be regulated (e.g., induced or repressed) in a eukaryotic environment (e.g., bacteria inside a eukaryotic organism or specific organ or structure in an organism). In some embodiments, the expression system promoter elements, isolated using methods described herein, can be selectively regulated. That is, the promoter elements sometimes can be influenced to increase transcription by providing the appropriate selective agent (e.g., administering tetracycline or kanomycin, metals, or starvation for a particular nutrient, for example, and described further below) to the host organism, such that the recombinant host cell containing the nucleic acid reagent comprising a selectable promoter element responds by showing a demonstrable (e.g., at least two fold, for example) increase in transcription activity from the promoter element.

In certain embodiments, an expression system may comprise a nucleotide sequence encoding a toxic or therapeutic RNA or protein or an RNA or protein that participates in generating a toxin or therapeutic agent operably linked to a promoter identified by the methods described herein. In some embodiments, an expression system as described herein may comprise a first promoter nucleotide sequence operably linked to a first coding sequence and a second promoter nucleotide sequence operably linked to a second coding sequence, where: the first coding sequence and the second coding sequence may encode RNA or polypeptides that individually do not inhibit tumor growth; RNA or polypeptides encoded by the first coding sequence and the second coding sequence, in combination, inhibit tumor growth; and the first promoter nucleotide sequence and the second promoter nucleotide sequence can be preferentially activated in solid tumors of living organisms. In some embodiments an expression system as described herein may comprise two or more sequences encoding toxic or therapeutic RNA or proteins, or RNA or proteins that participate in generating a toxin or therapeutic agent, operably linked to a similar number of promoter elements identified by methods described herein.

In some embodiments, a nucleotide coding sequence can encode an RNA that has a function other than encoding a protein. Non-limiting examples of coding sequences that do not encode proteins are tRNA, rRNA, siRNA, or anti-sense RNA. rRNA's (e.g., ribosomal RNA's) of various organisms sometimes have point mutations that confer antibiotic resistance. Expression of rRNA's that contain antibiotic resistance mutations inside a solid tumor, when the rRNA's are operably linked to a heterologous promoter sequence isolated using methods described herein, may provide a method for ensuring the survival of the recombinant cells only in the tumor environment, due to the resistance phenotype induced in the solid tumors. Therefore, all recombinant cells carrying the expression system would be susceptible to the antibiotic administered to the organism, except in the inside of the solid tumor.

In some embodiments, there is provided an expression system described above, where the first coding sequence can encode an enzyme, the second coding sequence can encode a prodrug, and the enzyme can process the prodrug into a drug that inhibits tumor growth. A non-limiting example of this type of combination is an inactive peptide toxin and an enzyme which cleaves the inactive form to release the active form of the toxin. Another example may be an antibody, whose protein sequence has been determined and a synthetic gene has been generated, and which requires processing (e.g., polypeptide cleavage) for assembly into an active form. In such examples, the first and second coding sequences are preferentially expressed inside the solid tumors, as the methods described herein select promoter elements preferentially activated in solid tumors. The combination of targeted, tumor specific expression, by delivery of the expression system comprising the nucleic acid reagent further comprising promoter elements preferentially activated in solid tumors of living organisms, as identified and isolated as described herein, and enzyme catalyzed activation of prodrugs, offers a significant improvement in gene-directed enzyme prodrug therapies. The expression systems described herein can be used to express prodrugs that, when activated, increase the bioavailability of therapeutic agents in solid tumor, or directly inhibit tumor growth by the action of the activated prodrug. In some embodiments, the second coding sequence can be a bacterial operon encoding a number of peptides, polypeptides or proteins which functionally form the prodrug. In some embodiments the first and second coding sequences can encode synthetically engineered enzymes or proteins specifically designed as prodrugs for anticancer therapies.

In some embodiments, there is provided an expression system, where the first coding sequence can encode a first polypeptide, the second coding sequence can encode a second polypeptide, and the first polypeptide and the second polypeptide form a complex that inhibits tumor growth. Non-limiting examples of two component protein or peptide toxins that can be used as therapeutic agents include Diphtheria toxin, various Pertussis toxins, Pseudomonas endotoxin, various Anthrax toxins, and bacterial toxins that act as superantigens (e.g., Staphylococcus aureus Exfoliatin B, for example). A combination of targeted, tumor specific expression, by delivery of an expression system comprising a nucleic acid reagent further comprising promoter elements preferentially activated in solid tumors as identified and isolated as described herein, and the use of two component protein or peptide toxins, offers a significant improvement in targeted, in situ delivery of anticancer therapies. Another example of a complex can include expressing two or more portions of an antibody (e.g., a light chain and a heavy chain), where the two or more portions can self assemble into a complex having antibody binding activity (e.g., antibody fragment).

In some embodiments, the promoter elements of the expression systems described herein (e.g., the first promoter nucleotide sequence, the second promoter nucleotide sequence, or both promoter nucleotide sequences) comprise (i) a nucleotide sequence of Table 2A, (ii) a functional promoter nucleotide sequence 80% or more identical to a nucleotide sequence of Table 2A, or (iii) or a functional promoter subsequence of (i) or (ii). That is, a functional promoter nucleotide sequences that is at least 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to a nucleotide sequence of Table 2A. The term “identical” as used herein refers to two or more nucleotide sequences having substantially the same nucleotide sequence when compared to each other. One test for determining whether two nucleotide sequences or amino acids sequences are substantially identical is to determine the percent of identical nucleotide sequences or amino acid sequences shared.

Sequence identity can also be determined by hybridization assays conducted under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C.

Calculations of sequence identity can be performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, often 60% or more, and more often 70% or more, 80% or more, 90% or more, or 100% of the length of the reference sequence. The nucleotides or amino acids at corresponding nucleotide or polypeptide positions, respectively, are then compared among the two sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences. Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available at the http address www.gcg.com), using either a Blossum 62 matrix or a PAM250 matrix, and a gap weight of 16, 14, 12, 10, 8, 6, or 4 and a length weight of 1, 2, 3, 4, 5, or 6. Percent identity between two nucleotide sequences can be determined using the GAP program in the GCG software package (available at http address www.gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

In some embodiments, the first promoter nucleotide sequence and the second nucleotide sequence can be in the same nucleic acid molecule (e.g., the same nucleic acid reagent, for example). In certain embodiments, the first promoter nucleotide sequence and the second nucleotide sequence can be in different nucleic acid molecule (e.g., different nucleic acid reagents, for example). In some embodiments, three or more promoters can be in the same nucleic acid molecule, and in certain embodiments, three or more promoters can be on different nucleic acid molecules. In some embodiments, an expression system may comprise functional promoter subsequences that are about 20 to about 150 nucleotides in length.

In some embodiments, the first promoter nucleotide sequence (e.g., promoter element) and the second promoter nucleotide sequence can be bacterial nucleotide sequences. In some embodiments, three or more promoter nucleotide sequences can be bacterial nucleotide sequences. In certain embodiments, the bacterial sequences are Enterobacteriaceae sequences, and in some embodiments, the Enterobacteriaceae sequences are Salmonella sequences. In some embodiments, the expression systems described herein are contained within recombinant host cells. In certain embodiments, the cells can be Enterobacteriaceae. In some embodiments, the Enterobacteriaceae can be Salmonella, and in certain embodiments, the Salmonella can be avirulent Salmonella.

Nucleic Acids

A nucleic acid can comprise certain elements, which often are selected according to the intended use of the nucleic acid. Any of the following elements can be included in or excluded from a nucleic acid reagent. A nucleic acid reagent, for example, may include one or more or all of the following nucleotide elements: one or more promoter elements, one or more 5′ untranslated regions (5′UTRs), one or more regions into which a target nucleotide sequence may be inserted (an “insertion element”), one or more target nucleotide sequences, one or more 3′ untranslated regions (3′UTRs), and a selection element. A nucleic acid reagent can be provided with one or more of such elements and other elements (e.g., antibiotic resistance genes, multiple cloning sites, and the like) can be inserted into the nucleic acid reagent before the nucleic acid is introduced into a suitable expression host or system (e.g., in vivo expression in host, or in vitro expression in a cell free expression system, for example). The elements can be arranged in any order suitable for expression in the chosen expression system.

In some embodiments, a nucleic acid reagent may comprise a promoter element where the promoter element comprises two distinct transcription initiation start sites (e.g., two promoters within a promoter element, for example). In some embodiments, a promoter element in a nucleic acid reagent may comprise two promoters. In certain embodiments, the promoter element may comprise a constitutive promoter and an inducible promoter, and in some embodiments a promoter element may comprise two inducible promoters. In certain embodiments a nucleic acid reagent may comprise two or more distinct or different promoter elements. In some embodiments, the promoters may respond to the same or different inducers or repressors of transcription (e.g., induce or repress expression of a nucleic acid reagent from the promoter element). A nucleic acid reagent sometimes can contain more than one promoter element that is turned on at specific times or under specific conditions.

A nucleic acid reagent sometimes can comprise a 5′ UTR that may further comprise one or more elements endogenous to the nucleotide sequence from which it originates, and sometimes includes one or more exogenous elements. A 5′ UTR can originate from any suitable nucleic acid, such as genomic DNA, plasmid DNA, RNA or mRNA, for example, from any suitable organism (e.g., virus, bacterium, yeast, fungi, plant, insect or mammal). The artisan may select appropriate elements for the 5′ UTR based upon the expression system being utilized. A 5′ UTR sometimes comprises one or more of the following elements known to the artisan: enhancer sequences, silencer sequences, transcription factor binding sites, accessory protein binding site, feedback regulation agent binding sites, Pribnow box, TATA box, −35 element, E-box (helix-loop-helix binding element), transcription initiation sites, translation initiation sites, ribosome binding site and the like. In some embodiments, a promoter element may be isolated such that all 5′ UTR elements necessary for proper conditional regulation are contained in the promoter element fragment, or within a functional sub sequence of a promoter element fragment.

A nucleic acid reagent sometimes can have a 3′ UTR that may comprise one or more elements endogenous to the nucleotide sequence from which it originates, and sometimes includes one or more exogenous elements. A 3′ UTR can originate from any suitable nucleic acid, such as genomic DNA, plasmid DNA, RNA or mRNA, for example, from any suitable organism (e.g., virus, bacterium, yeast, fungi, plant, insect or mammal). The artisan may select appropriate elements for the 3′ UTR based upon the expression system being utilized. A 3′ UTR sometimes comprises one or more of the following elements, known to the artisan, which may influence expression from promoter elements within a nucleic acid reagent: transcription regulation site, transcription initiation site, transcription termination site, transcription factor binding site, translation regulation site, translation termination site, translation initiation site, translation factor binding site, ribosome binding site, replicon, enhancer element, silencer element and polyadenosine tail. A 3′ UTR sometimes includes a polyadenosine tail and sometimes does not, and if a polyadenosine tail is present, one or more adenosine moieties may be added or deleted from it (e.g., about 5, about 10, about 15, about 20, about 25, about 30, about 35, about 40, about 45 or about 50 adenosine moieties may be added or subtracted).

A nucleic acid reagent that is part of an expression system sometimes comprises a nucleotide sequence adjacent to the nucleic acid sequence encoding a therapeutic agent or pharmaceutical composition that is translated in conjunction with the ORF and encodes an amino acid tag. The tag-encoding nucleotide sequence is located 3′ and/or 5′ of an ORF in the nucleic acid reagent, thereby encoding a tag at the C-terminus or N-terminus of the protein or peptide encoded by the ORF. Any tag that does not abrogate transcription and/or translation may be utilized and may be appropriately selected by the artisan.

A tag sometimes comprises a sequence that localizes a translated protein or peptide to a component in a system, which is referred to as a “signal sequence” or “localization signal sequence” herein. A signal sequence often is incorporated at the N-terminus of a target protein or target peptide, and sometimes is incorporated at the C-terminus. Examples of signal sequences are known to the artisan, are readily incorporated into a nucleic acid reagent, and often are selected according to the expression chosen by the artisan. A tag sometimes is directly adjacent to an amino acid sequence encoded by a nucleic acid reagent (i.e., there is no intervening sequence) and sometimes a tag is substantially adjacent to the amino acid sequence encoded by the nucleic acid reagent (e.g., an intervening sequence is present). An intervening sequence sometimes includes a recognition site for a protease, which is useful for cleaving a tag from a target protein or peptide. A signal sequence or tag, in some embodiments, localizes a translated protein or peptide to a cell membrane.

Examples of signal sequences include, but are not limited to, a nucleus targeting signal (e.g., steroid receptor sequence and N-terminal sequence of SV40 virus large T antigen); mitochondria targeting signal (e.g., amino acid sequence that forms an amphipathic helix); peroxisome targeting signal (e.g., C-terminal sequence in YFG from S. cerevisiae); and a secretion signal (e.g., N-terminal sequences from invertase, mating factor alpha, PHO5 and SUC2 in S. cerevisiae; multiple N-terminal sequences of B. subtilis proteins (e.g., Tjalsma et al., Microbiol. Molec. Biol. Rev. 64: 515-547 (2000)); alpha amylase signal sequence (e.g., U.S. Pat. No. 6,288,302); pectate lyase signal sequence (e.g., U.S. Pat. No. 5,846,818); precollagen signal sequence (e.g., U.S. Pat. No. 5,712,114); OmpA signal sequence (e.g., U.S. Pat. No. 5,470,719); lam beta signal sequence (e.g., U.S. Pat. No. 5,389,529); B. brevis signal sequence (e.g., U.S. Pat. No. 5,232,841); and P. pastoris signal sequence (e.g., U.S. Pat. No. 5,268,273)).

A nucleic acid reagent sometimes contains one or more origin of replication (ORI) elements. In some embodiments, a template comprises two or more ORIs, where one functions efficiently in one organism (e.g., a bacterium) and another functions efficiently in another organism (e.g., a eukaryote). A nucleic acid reagent often includes one or more selection elements. Selection elements often are utilized using known processes to determine whether a nucleic acid reagent is included in a cell. In some embodiments, a nucleic acid reagent includes two or more selection elements, where one functions efficiently in one organism and another functions efficiently in another organism.

Examples of selection elements include, but are not limited to, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic (e.g., Diphtheria toxin) or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like).

Nucleic acid reagents can comprise naturally occurring sequences, synthetic sequences, or combinations thereof. Certain nucleotide sequences sometimes are added to, modified or removed from one or more of the nucleic acid reagent elements, such as the promoter, 5′UTR, target sequence, or 3′UTR elements, to enhance or potentially enhance transcription and/or translation before or after such elements are incorporated in a nucleic acid reagent. Certain embodiments are directed to a process comprising: determining whether any nucleotide sequences that increase or potentially increase transcription efficiency are not present in the elements, and incorporating such sequences into the nucleic acid reagent. A nucleic acid reagent can be of any form useful for the chosen expression system.

In some embodiments, a nucleic acid reagent sometimes can be an isolated nucleic acid molecule which may comprise a recombinant expression system, which expression system can comprise a nucleotide sequence encoding a toxic or therapeutic RNA or protein, or an RNA or protein that participates in generating a toxin or therapeutic agent operably linked to a heterologous promoter which promoter is preferentially activated in solid tumors in living organisms. In some embodiments, the promoter sequence can be a naturally occurring nucleotide sequence. In certain embodiments, a nucleic acid reagent sometimes can be two or more isolated nucleic acid molecules which may comprise a recombinant expression system, which expression system can comprise two or more nucleotide sequences encoding toxic or therapeutic RNA's or proteins, or RNA's or proteins that participate in generating a toxin or therapeutic agent operably linked to two or more heterologous promoters which promoters is preferentially activated in solid tumors in living organisms. In some embodiments, the isolated nucleic acid of the recombinant expression system is a promoter nucleic acid. In certain embodiments, the promoter is an Enterobacteriaceae promoter, and in some embodiments, the promoter is a Salmonella promoter.

Promoters

A promoter element typically comprises a region of DNA that can facilitate the transcription of a particular gene, by providing a start site for the synthesis of RNA corresponding to a gene. Promoters often are located near the genes they regulate, are located upstream of the gene (e.g., 5′ of the gene), and are on the same strand of DNA as the sense strand of the gene, in some embodiments. A promoter often interacts with a RNA polymerase, an enzyme that catalyses synthesis of nucleic acids using a preexisting nucleic acid. When the template is a DNA template, an RNA molecule is transcribed before protein is synthesized. Promoter elements can be found in prokaryotic and eukaryotic organisms

A promoter element generally is a component in an expression system comprising a nucleic acid reagent. An expression system often can comprise a nucleic acid reagent and a suitable host for expression of the nucleic acid reagent. For example, an expression system may comprise a heterologous promoter operably linked to a toxin gene, carried on a nucleic acid reagent that is expressed in a bacterial host, in some embodiments. Promoter elements isolated using methods described herein may be recognized by any polymerase enzyme, and also may be used to control the production of RNA of the therapeutic agent or pharmaceutical composition operably linked to the promoter element in the nucleic acid reagent. In some embodiments, additional 5′ and/or 3′ UTR's may be included in the nucleic acid reagent to enhance the efficiency of the isolated promoter element.

Methods described herein can be used to identify a promoter preferentially activated in tumor tissue. In some embodiments the method comprises; (a) providing a library of expression systems each comprising a nucleotide sequence encoding a detectable protein operably linked to a different candidate promoter; (b) providing the library to solid tumor tissue and to normal tissue; (c) identifying cells from each tissue that show high levels of expression of the detectable protein; and (d) obtaining the expression systems from the cells that produce greater levels of detectable protein in tumor tissue as compared to normal tissue, and identifying the promoters of the expression system. In some embodiments, the method further comprises scoring the promoters identified in (d) (e.g., by detecting a detectable protein, GFP for example). In certain embodiments, the library is provided in recombinant host cells. In some embodiments, the library of DNA fragments ranged in size from about 25 base pairs to about 10,000 base pairs in length. In some embodiments, the fragments can be randomly sized fragments. In certain embodiments, the fragments can be an ordered set of specific sequences in a particular size range.

In some embodiments, the promoters are Salmonella promoters and the recombinant host cells are Salmonella. In certain embodiments, the candidate promoters are from bacteria, or are 80% or more identical to promoters from bacteria. That is, the candidate promoters can be at least 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to promoters from bacteria. In some embodiments, the bacteria are Enterobacteriaceae (e.g., Salmonella).

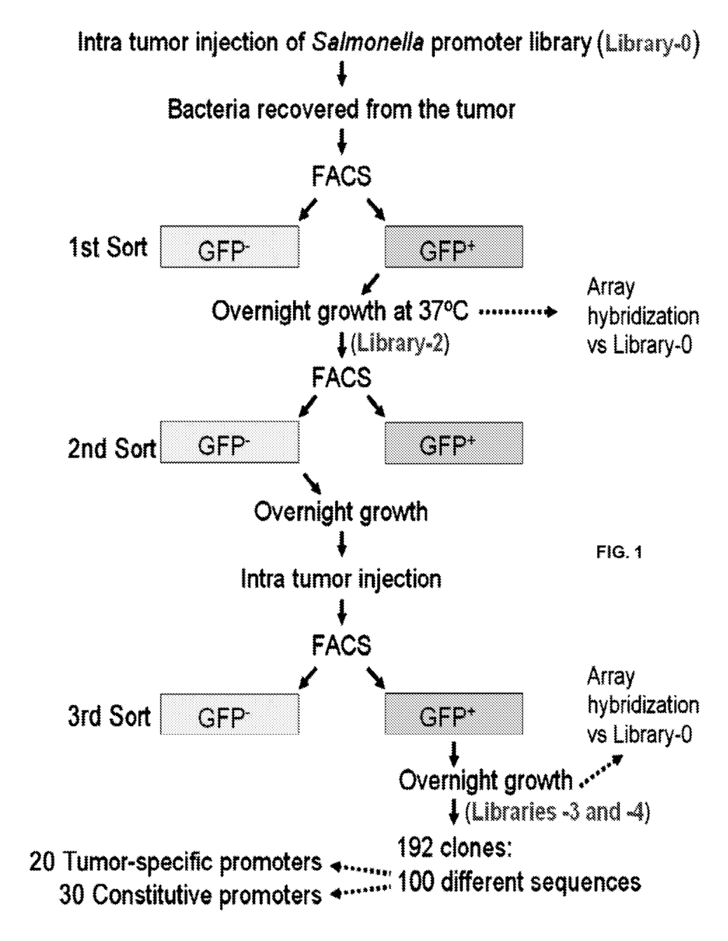

Detailed experimental procedures for construction of promoter trap constructs and libraries are presented below in Example 1 and in FIG. 1. FIG. 1 is a flow diagram outlining how the libraries were enriched for promoter sequences preferentially activated in solid tumors. The initial library was constructed by ligating sonicated, end repaired Salmonella genomic DNA, size selected for fragments 300 to 500 base pairs in length into a promoter trap construct upstream of a promoterless green fluorescent protein (GFP) sequence. Although GFP was the detectable protein used herein, due to ease of detection, any detectable protein that can be easily and efficiently detected can be used in place of GFP. Non-limiting examples of detectable proteins are other fluorescent proteins, peptides or proteins that inactivate antibiotics (e.g., beta-lactamase, the enzyme responsible for penicillin resistance, for example) and the like.

The library contained in recombinant cells can be injected into rodents (e.g., mice, rats) bearing solid tumor xenografts, as described below. Enrichment for promoters preferentially active in tumors was performed as described in Example 2. The experimental results from the enrichment process are presented in Tables 2-7. Tables 2-7 contain sequences of promoters active in normal tissue (e.g., spleen), promoters active in both normal tissue and solid tumors and promoters preferentially activated in solid tumors (see Tables 2A, 2B, 6A and 6B).

The sequences isolated using the methods described herein were mapped to genome positions as described in Example 2, using high density, high resolution arrays constructed as described in Example 1. The nucleotide position of the library construct that had the highest enrichment signal for a particular library construct is given in the Tables as the nucleotide position. The nucleotide position may correspond to the start site of the isolated promoter element. Definitive promoter start site mapping can be performed using a suitable method. One method is 5′ RACE (e.g., rapid amplification of cDNA ends), for example, which can be routinely performed. 5′ RACE can be used to identify the first nucleotide in an mRNA or other RNA molecule and also be used to identify and/or clone a gene when only a small portion of the sequence is known. An example of a 5′ RACE procedure suitable for identifying a transcription start site from promoter elements isolated using the methods described herein is Schramm et al, “A simple and reliable 5′ RACE approach”, Nucleic Acids Research, 28(22):e96, 2000.

Where identifiable, gene names and functions are presented along with the sequence information for the isolated nucleic acid sequences that exhibited promoter activity (e.g., showed at least a two fold increase in detectable GFP over input). Table 6 describes the distribution of sequences isolated using the methods described herein. The majority of sequences that exhibited promoter activity (e.g., transcription of GFP) were isolated from intergenic sequences. This observation is in keeping with the finding that many bacterial promoters lie outside of gene coding sequences. Further distribution results are discussed in Example 2.

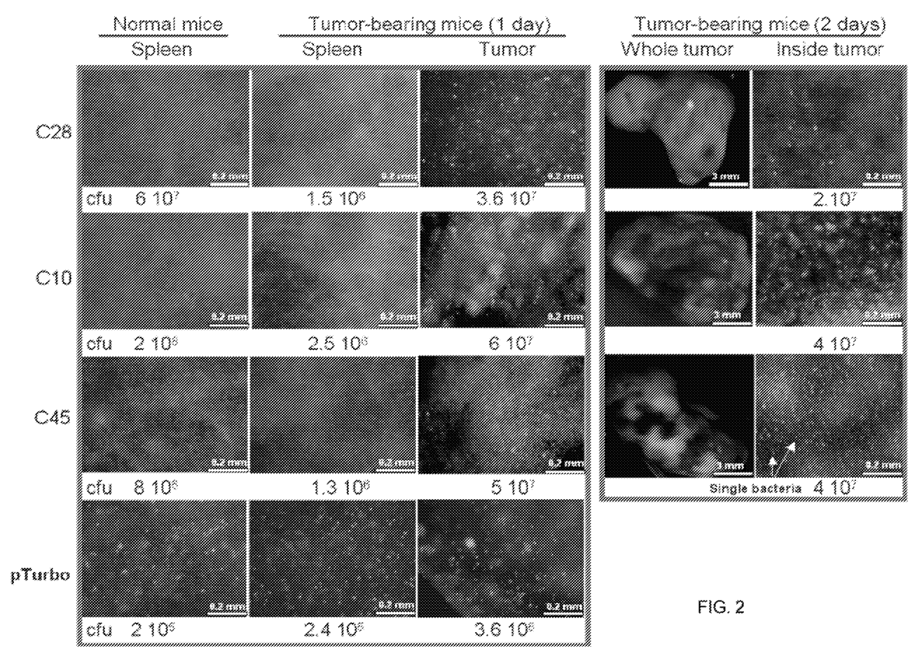

To confirm the tumor specificity of the isolated sequences, a number of clones were further investigated (see Example 2, Confirmation of tumor specificity in vivo). In particular, Clone ID Nos. 10, 28, 45, 44, and 84 were further investigated in vivo as described in Example 2. Three clones in particular were induced to a greater degree in tumor as compared to spleen (e.g., Clones 10, 28 and 45). FIG. 2 illustrates the expression of GFP from these clones in vivo in whole mice and in tumor alone. FIG. 2 presents the microscopic imaging (Olympus OV100 small animal imaging system) of fluorescent bacteria in mouse spleen and tumors. Clone C28 maps to the upstream intergenic region of the flhB gene, clone C10 maps to the pefL intergenic region, and C45 maps to the intergenic region of the gene ansB. The number of colony forming units for each trial is given below the image, to account for differences in signal intensities. The number of colony forming units isolated in each trial was approximately equal, and therefore did not contribute to the differences in intensity seen in the images.

Certain promoter elements can be regulated in a conditional manner. That is, promoters sometimes can be turned on, turned off, up-regulated or down-regulated by the influence of certain environmental, nutritional, or internal signals (e.g., heat inducible promoters, light regulated promoters, feedback regulated promoters, hormone influenced promoters, tissue specific promoters, oxygen and pH influenced promoters and the like, for example). Promoters influenced by environmental, nutritional or internal signals frequently are influenced by a signal (direct or indirect) that binds at or near the promoter and increases or decreases expression of the target sequence under certain conditions and/or in specific tissues. Certain promoter elements can be regulated in a selective manner, as noted above. In some embodiments, the promoter does not include a nucleotide sequence to which a bacterial (e.g., gram negative (e.g., E. coli, Salmonella) oxygen-responsive global transcription factor (FNR) binds substantially. In certain embodiments, the promoter sequence does not include one or more of the following subsequences:

GGATAAAAGTGACCTGACGCAATATTTGTCTTTTCTTGCTTAATAATGTT
GTCA,
GGATAAAAGTGACCTGACGCAATATTTGTCTTTTCTTGCTTTATAATGTT
GTCA,
GGATAAAATTGATCTGAATCAATATTTGTCTTTTCTTGCTTAATAATGTT
GTCA,
or
GGATAAAAGGATCCGACGCAATATTGTCTTTTCTTGCTTAATAATGTTGT
CA.

In some embodiments, the promoter sequence is not identical to a bacterial promoter that regulates the bacterial pepT gene.

Non-limiting examples of selective agents that can be used to selectively regulate promoters in therapeutic methods using expression systems and promoter elements described herein include, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic (e.g., Diphtheria toxin) or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like). In some embodiments, the nucleic acids identified and isolated using methods described herein (e.g., promoter elements preferentially activated in solid tumors of living organisms) can be selectively regulated by administration of a suitable selective agent, as described above or known and available to the artisan.

Methods presented herein take into account the unique environment inside a tumor. Therefore, while hypoxia induced tumors may be identified, other promoters preferentially activated in the unique tumor environment can also be identified and isolated. Some specific classes of promoters preferentially activated inside tumors were presented above. Therefore, the promoters isolated using methods described herein may be preferentially activated under a wide variety of regulatory molecules and conditions.

Therapeutic Agents and Methods of Treatment

Expression systems, nucleic acid reagents and pharmaceutical compositions described herein that comprise promoter elements preferentially activated in solid tumors, or cells containing the expression system, nucleic acid reagents and pharmaceutical compositions described herein, can be used to treat solid tumors in a living organism. In some embodiments, methods for treating solid tumors comprise administering to a subject harboring the tumors the nucleic acid molecules or nucleic acid reagents comprising nucleic acid sequences preferentially activated in tumors (e.g., nucleic acids bearing promoter elements isolated using the methods described herein, for example), cells containing the above described nucleic acids, or compositions comprising the isolated nucleic acids. In some embodiments, the expression system, nucleic acid reagent, and/or pharmaceutical compositions comprise a nucleotide sequence encoding a toxic or therapeutic RNA or protein, or an RNA or protein that participates in generating a desired toxin or therapeutic agent operably linked to a promoter identified by the methods described herein.

In some embodiments, the therapeutic RNA or protein can be an enzyme which catalyzes the activation of a prodrug. That is, the enzyme can be operably linked to a promoter element preferentially activated in solid tumors. The nucleic acid reagent/expression system/pharmaceutical composition contained in a recombinant cell can be administered along with the prodrug (e.g., administered by intramuscular or intravenous injection, for example). The avirulent recombinant host cell sometimes can preferentially colonize the solid tumor, and the prodrug will remain inactive in all tissues except inside the solid tumor, due to the enzyme only being produced by recombinant cells that have colonized the tumor, due to the heterologous promoter that is preferentially activated in the solid tumors of living organisms. Non-limiting examples of this type of combination are the enzymes nitroreductase or quinone reductase 2 and the prodrug CB1954 (5-[aziridin-1-yl]-2,4-dinitrobenzamide), or Cytochrome P450 enzymes 2B1, 2B4, and 2B5 and the anticancer prodrugs Cyclphosphamide and Ifosfamide. Further non-limiting examples of enzyme prodrug combinations can be found in Rooseboom et al, “Enzyme-Catalyzed Activation of Anticancer Prodrugs”, Pharmacol. Rev. 56:53-102, 2004, hereby incorporated by reference in its entirety.

In certain embodiments, bacterial two component toxins can also be utilized as the toxic or therapeutic proteins or peptide sequences operably linked to the promoters isolated using methods described herein. Non-limiting examples of bacterial toxins suitable for use in compositions described herein were presented above. Several of these toxins offer attractive modes of toxicity that when combined with the expression only inside a solid tumor, may offer novel therapies for inhibiting tumor growth. For example, Diphtheria toxin and Pseudomonas Exotoxin A are both two component toxins (e.g., has two distinct peptides) that inhibit protein synthesis, resulting in cell death. The nucleic acid sequences of these toxins could be operably linked to promoters preferentially activated in solid tumors, and administered to a subject harboring a solid tumor, with little or no toxicity to the organism outside of the targeted solid tumor.

In some embodiments, multiple nucleic acid reagents can be administered, where each nucleic acid reagent comprises a nucleic acid sequence for a gene in a metabolic pathway, the pathway producing a therapeutic agent that can inhibit tumor growth. In certain embodiment the nucleic acid reagents can have the same or different heterologous promoters preferentially activated in tumors operably linked to the sequences for the metabolic pathway genes.

In certain embodiments, the expression systems described herein may generate RNA's or proteins that are themselves toxic, or RNA's or proteins that are known to have a therapeutic effect by selective toxicity to solid tumors. A non-limiting example of a protein known to have a therapeutic effect by selective toxicity to solid tumors is Methioninase, which is known to be selectively inhibitory to tumors. Additional known toxic proteins include, but are not limited to, ricin, abrin, and the like. In addition to proteins that are toxic per se, the expression systems may generate proteins that convert non-toxic compounds into toxic ones. A non-limiting example is the use of lyases to liberate selenium from selenide analogs of sulfur-containing amino acids. Other non-limiting examples include generation of enzymes that liberate active compounds from inactive prodrugs. For example, derivatized forms of palytoxin can be provided that are non-toxic and the expression system used to produce enzymes that convert the inactive form to the toxic compound. In addition, proteins that attract systems in the host can also be expressed, including immunomodulatory proteins such as interleukins.

The subjects that can benefit from the embodiments, methods and compositions described herein include any subject that harbors a solid tumor in which the promoter operably linked to a therapeutic agent is preferentially active. Human subjects can be appropriate subjects for administering the compositions described herein. The methods and compositions described herein can also be applied to veterinary uses, including livestock such as cows, pigs, sheep, horses, chickens, ducks and the like. The methods and compositions described herein can also be applied to companion animals such as dogs and cats, and to laboratory animals such as rabbits, rats, guinea pigs, and mice.

The tumors to be treated include all forms of solid tumor, including tumors of the breast, ovary, uterus, prostate, colon, lung, brain, tongue, kidney and the like. Localized forms of highly metastatic tumors such as melanoma can also be treated in this manner.

Thus, the methods and compositions described herein may provide a selective means for producing a therapeutic or cytotoxic effect locally in tumor or other target tissue. As the encoded RNA's or proteins are produced uniquely or preferentially in tumor tissue, side effects due to expression in normal tissue is minimized.

Nucleic acid molecules may be formulated into pharmaceutical compositions for administration to subjects. The nucleic acid molecules sometimes are transfected into suitable cells that provide activating factors for the promoter. In some cases, the tumor cells themselves may contain workable activators. If the promoter is a bacterial promoter, bacteria, such as Salmonella itself, may be used. Any cell closely related to that from which the promoter derives is a suitable candidate. A preferred mode of administration is the use of bacteria that preferentially reside in hypoxic environments of solid tumors. The compositions which contain the nucleic acids, vectors, bacteria, cells, etc., sometimes are administered parenterally, such as through intramuscular or intravenous injection. The compositions can also be directly injected into the solid tumor. Nucleic acids sometimes are administered in naked form or formulated with a carrier, such as a liposome. A therapeutic formulation may be administered in any convenient manner, such as by electroporation, injection, use of a gene gun, use of particles (e.g., gold) and an electromotive force, or transfection, for example. Compositions may be administered in vivo, ex vivo or in vitro, in certain embodiments.

As noted above, ancillary substances may also be needed such as compounds which activate inducible promoters, substrates on which the encoded protein will act, standard drug compositions that may complement the activity generated by the expression systems of the invention and the like. These ancillary components may be administered in the same composition as that which contains the expression system or as a separate composition. Administration may be simultaneous or sequential and may be by the same or different route. Some ancillary agents may be administered orally or through transdermal or transmucosal administration.

The pharmaceutical compositions may contain additional excipients and carriers as is known in the art. Suitable diluents and carriers are found, for example, in Remington's Pharmaceutical Sciences, latest edition, Mack Publishing Co., Easton, Pa., incorporated herein by reference.

### EXAMPLES

The examples set forth below illustrate certain embodiments and do not limit the invention.

### Example 1

### Materials and Methods

Vector Construction.

Promoter trap plasmids with TurboGFP (e.g., promoter reporter plasmid comprising a destabilized TurboGFP, World Wide Web URL evrogen.com/TurboGFP.shtml) were generated by PCR from the pTurboGFP plasmid. The pTurboGFP plasmid was PCR amplified using the primers Turbo-LVA R1 (SEQ ID NO. 1, see Table 1) and Turbo-F1 (SEQ ID NO. 2, see Table 1) to generate a fusion of the peptide motif AANDENYALVA (SEQ ID NO. 3) to the 3′ end of the protein (Andersen et al., 1998; Keiler and Sauer, 1996). The PCR product was digested by EcorRV and self ligated to generate pTurboGFP-LVA. The plasmids pTurboGFP and pTurboGFP-LVA were each double digested by XhoI and BamH1 to remove the T5 promoter sequence. The pairs of oligos PR1-1F/PR1-1R (SEQ ID NOS. 4 and 5, respectively, see Table 1) and PRL3-1F/PR3-1R (SEQ ID NOS. 6 and 7, respectively, see Table 1), containing multi-cloning sites, transcriptional terminators, and a ribosomal binding site, were used to replace the T5 constitutive promoter of pTurbo-GFP and pTurboGFP-LVA respectively. Primers Turbo-4F and Turbo-1R (SEQ ID NOS. 8 and 9, respectively, see Table 1) were used to amplify promoter inserts before and after FACS sort.

TABLE 1

Sequences of oligonucleotides use to construct
promoter trap constructs

| | |
|---|---|
| Oligos | Sequence |

| | |
|---|---|
| Turbo-LVA R1 | SEQ. ID. NO. 1: |
| | ACTGATATCTTAAGCTACTAAAGCGTAGTTTTCGTCGTTTGCTGCAGGCCTT |
| | TCTTCACCGGCATCTGCA |
| T urbo-F1 | SEQ. ID. NO. 2: CTGATATCGCTTGGACTCCTGTTGATAGAT |
| PRL1-1F | SEQ. ID. NO. 4: |
| | TCGAGAGATCTCCATCGAATTCGTGGGTCGACCCCGGGAGGCCTAAAGAG |
| | GAGAAATTAACTATGAGAGGATCGG |
| PRL1-1R | SEQ. ID. NO. 5: |
| | GATCCCGATCCTCTCATAGTTAATTTCTCCTCTTTAGGCCTCCCGGGGTCGA |
| | CCCACGAATTCGATGGAGATCTC |
| PRL3-1F | SEQ. ID. NO. 6: |
| | TCGAGCGAAATTAATACGACTCACTATAGGGAGACCCCCGGGTTAACACTA |
| | GTAAAGAGGAGAAATTAACTATGAGAGGATCGG |
| PRL3-1R | SEQ. ID. NO. 7: |
| | GATCCCGATCCTCTCATAGTTAATTTCTCCTCTTTACTAGTGTTAACCCGGG |
| | GGTCTCCCTATAGTGAGTCGTATTAATTTCGC |
| Turbo-4F | SEQ. ID. NO. 8: AAAGTGCCACCTGACGTCT |
| Turbo-1R | SEQ. ID. NO. 9: CCACCAGCTCGAACTCCAC |

Promoter Library Construction.

10 μg of Salmonella enterica serovar typhimurium 14028 (S. enterica. Typhimurium 14028, ATCC) genomic DNA was eluted in TE buffer and sonicated with 3 pulses for 5 seconds on ice. Sonicated DNA was precipitated with 2 volumes ethanol and 0.1 volumes of Sodium Acetate (100 mM) and separated on a 1% agarose gel. 300 to 500 base pair (bp) fragments were recovered from the gel and DNA ends were repaired by T4 DNA polymerase. Repaired fragments were cloned in a dephosphorylated promoterless GFP plasmid upstream of a StuI and HpaI restriction site in the stable and destabilized GFP, respectively. These fragments were located just upstream of the GFP start codon, and were therefore capable of promoting transcription, depending on their sequence properties. The number of independent clones was approximately 120,000 for the stable variant and 60,000 for the unstable variant. The two libraries were mixed 1:1 and designated “Library-0”. This library contained about 180,000 independent Typhimurium fragments, representing about 15-fold coverage of the 4.8 Mb genome with clone spacing averaging every 25 bases. Hybridization to a Salmonella array showed that library-0 represented sequences from almost the entire genome.

Array Design.

A high-resolution array was generated using Roche NimbleGen high definition array technology (World Wide Web URL nimblegen.com/products/index.html). The array comprised 387,000 46-mer to 50-mer oligonucleotides, with length adjusted to generate similar predicted melting temperatures (Tm). 377,230 of these probes were designed based on the Typhimurium LT2 genome (NC-003197; McClelland et al, “Complete genome sequence of Salmonella enterica serovar Typhimurium LT2”, Nature 413:852-856, 2001). Oligonucleotides tiled the genome every 12 bases, on alternating strands. Thus, each base pair in the genome was represented in four to six oligonucleotides, with two to three oligonucleotides on each strand. Probes representing the three LT2 regions not present in the genome of the very closely related 14028s strain (phages Fels-1 and Fels-2, STM3255-3260) and greater than 9,000 other oligonucleotides were included as controls for hybridization performance, synthesis performance, and grid alignment. The oligonucleotides were distributed in random positions across the array.

Fluorescence Activated Cell Sorting (FACS) Analysis.

Bacteria harboring the constitutive pTurboGFP plasmid were used as a positive control for the Becton Dickinson FACSAria FACS system. Side scatter ssc-w (X-axis) and ssc-H(Y-axis) were used to gate on single bacterial cells. GFP-fluorescence (GFP-A) on the X-axis and auto-fluorescence (PE) on the Y-axis permitted discrimination between green Salmonella cells and other fluorescent particles of different sizes. Fluorescent particles tended to be distributed on the diagonal of the GFP-A/PE plot, and had a fluorescence/auto-fluorescence ratio close to 1. Individual GFP-positive Salmonella cells had a higher ratio of fluorescence/auto-fluorescence and tended to be distributed close to the X-axis of the GFP-A/PE plot. Putative GFP-positive events in the window enriched for GFP-expressing Salmonella were sorted at a speed of ‘5,000 total events per second.

### Example 2

### Experimental Results

Enrichment of Active Promoters in Spleen.

To identify active Salmonella promoters in the spleen, five tumor-free nude mice were i.v. injected with 10⁷ colony forming units (cfu) of Salmonella carrying a promoter library. This library, designated “library-0”, consisted of ˜180,000 plasmid clones each containing a fragment of the Salmonella genome upstream of a promoterless GFP gene (described above). Two days after injection, spleens were combined, homogenized on ice, and treated thrice with PBS containing 0.1% Triton X-100. An aliquot of the final homogenized sample was plated on Luria-Bertani (LB) medium with 50 μg/mL of ampicillin (Amp) to determine the number of bacterial colony-forming units (cfu). The remainder of the bacteria in the sample was immediately separated by FACS. Fifty thousand potentially GFP-positive events were sorted and this sublibrary was grown overnight in LB+ Amp and designated “library-1”. The spleen was chosen because it is the primary site of Salmonella accumulation in normal mice (Ohl and Miller, “Salmonella: a model for bacterial pathogenesis”, Annu. Rev. Med. 52:259-274, 2001).

Enrichment of Active Promoters in Tumor.

The experimental design for tumor samples is described in FIG. 1. Five nude mice bearing human-PC3 prostate tumors, between 0.5 and 1 cm³ in size, were injected intratumorally with 10⁷ cfu of Salmonella promoter library-0. Two days after injection, tumors were combined, homogenized on ice and washed, as above. An aliquot was plated to determine the number of bacterial colony-forming units. The remainder of the sample was immediately separated by FACS. Fifty thousand GFP-positive events were recovered and grown overnight in LB containing ampicillin (library-2). A small aliquot of these bacteria were then pelleted and resuspended in PBS (10⁶ cfu/mL) and FACS sorted. GFP-negative events (10⁶) were collected, grown in LB overnight, washed in PBS and reinjected into five human-PC3 tumors in nude mice. After 2 days, bacteria were extracted from tumors and 50,000 GFP-positive events were FACS sorted and expanded in LB+ Amp (library-3). A biological replicate of library-3 was obtained by repeating the experiment from the beginning using library-0. This was designated library-4.

Genome wide Survey on Tumor-Activated Promoters Using Arrays.

Plasmid DNA was extracted from the original promoter library (library-0), from clones activated in spleen (library-1), and from clones activated in subcutaneous PC3 tumors in nude mice after one (library-2) or two passages (library-3 and library-4) in tumors. Promoter sequences were recovered by PCR using primers Turbo-4F and Turbo-1R (see Table 1, presented above), and the PCR product was labeled by CY 5 (library-0) and CY 3 (library-1, library-2, library-3, library-4). The resulting products were then hybridized to the array of 387,000 oligonucleotide sequences (described above in Array Design) positioned at 12-base intervals around the Typhimurium genome (using the manufacturer's protocol) (Panthel et al, “Prophylactic anti-tumor immunity against a murine fibrosarcoma triggered by the Salmonella type III secretion system”, Microbes Infect. 8:2539-2546, 2006). Spot intensities were normalized based on total signal in each channel. The enrichment of genomic regions was measured by the intensity ratio of the tumor or the spleen sample versus the input library (library-0). A moving median of the ratio of tumor versus input library from 10 data points (−170 bases) was calculated across the genome.

The highest median of each intergenic and intragenic region was chosen to represent the most highly overrepresented region of that promoter or gene in the tested library. Using a threshold of (exp/control) greater than or equal to 2, and enrichment in both replicates of the experiment (library-4, plus at least one of library-2 or library-3), there were 86 intergenic regions enriched in tumors but not in the spleen (see Table 2A and 2B, presented below), and 154 intergenic regions enriched in both tumor and spleen (see Table 3A and 3B, presented below). There were at least 30 regions enriched in spleen alone (see Table 4, presented below).

TABLE 2A
Intergenic regions that induce higher GFP expression in tumor than in spleen

Median ratio of experiment
versus input

| | | | | | | |
|---|---|---|---|---|---|---|
| | Genome | | | | Tumor | Tumor |
| Inter- | position | Arbitrary | | Tumor | (+) | (+) |
| genic | of peak | clone | Spleen | (+) | (−)(+) | (−)(+) |
| region | signal | number | Lib-1 | Lib-2 | Lib-3 | Lib-4 |

| | | | | | | | |
|---|---|---|---|---|---|---|---|
| STM0468- | 526177 | 85 | 0.9 | 2.3 | 5.5 | 9.5 | TCAACTTGACGGTGCGCCAGCCACAGACTCAATCCTATCGGGAAA |
| STM0469 | | | | | | | AGGACAGACAGGATAAGCACTCCCGTTACCAGGCTGACCAGATGT |
| | | | | | | | CGTGTTGTCACAGTGATGTCCTTATAAACACAGCGTAGAGAAAGTA |
| | | | | | | | TATCCGATCGTAAATCGCGCCCTCGAATGATAAAGCTATTTTATCG |
| | | | | | | | ATTTTACAGATTCAGGCGCCAGGCTAACGCGTTACGCCACGTTGCT |
| | | | | | | | TTTGCCGCCAGGAAGAGATCGTGAATGTTTACCGGTTGAAAAAGG |
| | | | | | | | AGCGTTGATAGCGTATTTTATTGTTATG |
| STM0474- | 529126 | 86 | 1.9 | 1.7 | 3.2 | 2.6 | TATTGTTTGTGTAATCATTGGGTTAACGTTTTTTAGCTTTTCAGGCTA |
| STM0475 | | | | | | | AAACAATAGACTCTGACAGGAGAAAATAGCCAGGAATATTCTTAAT |
| | | | | | | | ATTTCTTAATTAATGGCTGAATTAAGAAATGGCCAACTTTCCTAAGA |
| | | | | | | | AAAGCCTTTAACGCAGTAAGGATTATACCTTTTATTAATATGGCAAA |
| | | | | | | | AAATAATCAATCTAACAATAAGCGTATTTTATGATTTTTGCGTAAAA |
| | | | | | | | AAGGCCGCTTGCGCGGCCTTATCAACAGTGAGCAAATCAGCGATG |
| | | | | | | | TTCTGTCGAATGACTATGCTC |
| STM0580- | 638735 | 87 | 0.9 | 3.2 | 0.3 | 8.5 | AAATAGCGAAACAATGTTCCTTCTGCAACACCTGCGTTACGCGCAA |
| STM0581 | | | | | | | TCACCGCCGTTGAGGCGGCGATACCGGATTGCGCTATCGCCTGGG |
| | | | | | | | TTGCCGCTTCCAGTAATGCTTGTTTTTTGTCTTCACTCTTCGGACGA |
| | | | | | | | GCCACTACACGTTACCCTTATGTCTGGAAAAACATGATTGAATCAT |
| | | | | | | | GCCCGTTGTCGCGTCGCAACGGTGAATGTCAACCTTTGAAAAGTAC |
| | | | | | | | CTTGACGGCGTATCTTTGCTTTCTATAATGAGTGCTTACTCACTCAT |
| | | | | | | | AATCAAGGGCTGCCGCATGAAGTG |
| STM0844- | 914762 | 10 | 0.8 | 1.9 | 5.8 | 0.4 | AGCCTTTGAGAAATACTACGGTACGGATACCGGGGCCATCGTGGG |
| STM0845 | | | | | | | TAGAATAGCGCTGAATATTGAAGATCATAAACGGCCTCTCTTATTT |
| | | | | | | | CATATAAAGATTAAATTACTTTCGAATGAAAGCTATCTTGATGTGCG |
| | | | | | | | TCAACGAATGGAGAGGTTCTGACAAAGAGGCGTTAAATGAGGTAC |
| | | | | | | | AACATCACGGTTTGAGGTTGTGGTATGGCGTTTAAGATGATGCCGC |
| | | | | | | | GCTGCTTGAGCCGATCGTCAGTCGGAGCTTGGGTAAGCTGGCTTT |
| | | | | | | | GCGTCTGATGACAGTAATTATCTGTTG |
| STM0937- | 1014704 | 11 | 0.7 | 4.2 | 6.5 | 10.3 | GCGTAGGAGCAGCCGTTTCCGGCTGGTGTACGGATGGTTTGTTCA |
| STM0938 | | | | | | | CATTGCACACAAAACATGGTCACACCTTTTAAAGTTATATTTAATAT |
| | | | | | | | ACATGTTTAAGGTTATGCCTGTGAACAAAGGGATAAAAGGGATTTC |
| | | | | | | | TGCCATAATGTGCAGGGAGATTGATTTAGCGCAATTTTGGCGGCAG |
| | | | | | | | ATGCCTACCGCCAAAGAGGTATCAGGCCGAGAAGAACGCCATTAA |
| | | | | | | | GAGGGGGACCAGCAGGCTGAGGATAAAGCCATGTACGATAGCCG |
| | | | | | | | CCGGAACAATCTCTACGCCGCCGGAGCG |
| STM1382- | 1466034 | 16 | 0.7 | 4.6 | 7.4 | 13.9 | TGAAGCATACCTGATTTCTGGAAATAGCGTAGATCGGAACGAATAG |
| STM1383 | | | | | | | TCTCCTGGCTAACCTTATAAAGGTCTGAAAGTTTACTGACGCTAAC |
| | | | | | | | ACTATTATCCTTTATCAGTAAATTAATGATGGCATGACGTCTTTCTT |
| | | | | | | | CTTTAAACATATTGCCTCCGGGTAGTGAGTTGAATTGTATTTATGGC |
| | | | | | | | AATGTTGTCATGCGGTGAATTCAATCACAGATTATGCGGTCAACCG |
| | | | | | | | GAAGTAACCCCAAATGAATGTCAATAATCAGAAGCGCAGCCAATG |
| | | | | | | | TGTTAAATATTAATTGCTTACAGA |
| STM1529- | 1606103 | 20 | 1.9 | 5.5 | 2.8 | 13 | TACACAAATGACCGTTTGCGCTATGTGATAATTAACCATAGTAAAA |
| STM1530 | | | | | | | ATACACGAAGCGAAGAAGTGCTATTTCAGTAGTACTGATATTTTCA |
| | | | | | | | TAACGCTAATTTAAAAATAAATGTAAACGTAACAAATTATACACAA |
| | | | | | | | AAATAAGAAGGGCTGTGGCCTCAACTGACTGGATTATGATTCCGTC |
| | | | | | | | TTACCGAATGTCAGCCGAATGTTCAGTGCCATTCTCGCCCTGGCAT |
| | | | | | | | CCCCGACCGTAAGCCTGTTCTCTACTGGTAACCCCCTTGTTATTAC |
| | | | | | | | AGCAGAAAACAGGGCATATCATTGA |
| STM1807- | 1909051 | 26 | 1.2 | 1.6 | 6.5 | 9.7 | TGCGCCGAACGCCAGTGGTCGTTTTTAACGCTGGAGATGCCGCAA |
| STM1808 | | | | | | | TGGCTGTTGGGGATCTTTGCCGCTTACCTTGTGGTGGCGATAGCCG |
| | | | | | | | TCGTCATAGCCCAGGCATTTAAGCCTAAAAAACGCGACCTGTTCGG |
| | | | | | | | TCGTTGATACACACGCTCCTTCGGGAGCGTTTTTTTTGCCCGAAGC |
| | | | | | | | GTTGTTTGCCAGTGATTAAAAGGTGTATATTAAATACATCTTTTAAT |
| | | | | | | | CACCACATCAGGGAGATGTCTTATGTCCCACTTACGCATCCCGGCA |
| | | | | | | | AACTGGAAAGTTAAACGCTCTACCC |
| STM1914- | 2011503 | 28 | 0.9 | 3.9 | 7.2 | 7.5 | GGATCTGCCCTTCTTCCCGCGCTTTTTCAAGTCGGTGGGGTGTGGG |
| STM1915 | | | | | | | GGCTTCTGTTTTGTCGTCGTCGCTCTCTTCTGCCACGCAGCAAACC |
| | | | | | | | CTGGATAGATTGATAAGAGAGAATGATGCCAGAACCGCTTTACGC |
| | | | | | | | CAATAGGCAGAGTAAGCGGTAAAAAAGGCGGGGTTTATGGCGTTA |
| | | | | | | | ATAGAGATAGCCGGATACGATAAGAAAGTCTCGTATCCGGCCGGG |
| | | | | | | | TTGACGGATTCGAACCCGATAAGCGCAGCGCCATCAGGTCAAAAA |
| | | | | | | | AGCTTAAAAGCCAAGACTGTCCAGCAGGT |
| STM1996- | 2079476 | 30 | 1.2 | 2.9 | 7.4 | 4 | GAATGGCTGAAAAATGCACAAACACATCTTTGCTGCCATCTTTAGG |
| STM1997 | | | | | | | CGTAATGAAACCAAAGCCCTTTTCAGGGTTAAACCATTTTACTAAA |
| | | | | | | | CCAGTGATTTTCGTCGTCATAATATTGTTACCTTTCGAATGAGCCCT |
| | | | | | | | TGGGCAAAATGGCCTGAAGAAAATTATCAGAGAGAAAAAAACCTA |
| | | | | | | | AAGGAGATCTCAAGAGGAACAAATGATGAGAAATATTACAATCAC |
| | | | | | | | TACTTCAGATAAGTTTGTATCAAACCGCACAACCATTAACGCATGG |
| | | | | | | | TTAACTGAACATAGCAAGCTTTAGTT |
| STM2035- | 2114187 | 31 | 1.3 | 5.9 | 4.7 | 8 | ACCACAAATGTGGCAAACCTGTTGGTTTACGTTATGGCTGTACGGC |
| STM2036 | | | | | | | ACACCCATAACGACAATTAATAATGTGCTACGTTTTACATTTCTGTG |
| | | | | | | | AGCAATAGCCTGAGCGGTTGCTCATCTGACGTTAATCTACTCATCC |
| | | | | | | | TTACCGGTATATTGACGATAAAACGTATCGACAAAGCGTAATAAAA |
| | | | | | | | CTTATCTTTCCTGACACTGTACTTCATCACAAAAATAAAAACTGGTG |
| | | | | | | | CAGTTTATGCCCTAAATTTTATTATTTTGTTGCGCTATGACAATTTAT |
| | | | | | | | TGTTACACCAGATAAATTTTC |
| STM2261- | 2359663 | 34 | 0.6 | 2.1 | 3.5 | 4.8 | CCTGGATGCAGGCGTCGCAACGCAGACAATGTGCGAGAAAATAGG |
| STM2262 | | | | | | | TCGTTTCTCTGGCCCACGGCGGAAGAATCCCATTGCTGGCGTTGCG |
| | | | | | | | CCAACTGCCGGTCAACATGCTTCGACGGGATAAATCAACCATGAT |
| | | | | | | | ATCGCCCTTCCATAACGACACGCTTCCATAGGGAGTGAATACCAAT |
| | | | | | | | AAAAACCGTACAATTTATGAGTAGTTGTTTTTGTAAATAAGATATTT |
| | | | | | | | CAGGATGTGTAAGAGATGCATACCCCGATAGAGGTAAATGCTGTT |
| | | | | | | | GCCGGATCAAAAGAGTGCCGGGTAAAG |
| STM2309- | 2417301 | 36 | 0.6 | 2.7 | 6.5 | 6.3 | TGAATAAAAGCAGGATTCTCTGCCGCCGCCAACGTGAGCGGCGTG |
| STM2310 | | | | | | | GAACGGGAACCAGGGGCGATACAAACATGCCTGACGCCATGACG |
| | | | | | | | GGTTAAGGCTTCCAGGATGACCGCCGCCCAGCGCCGGTTAAATGC |
| | | | | | | | ACTTACTGACATGAGTTTGTCCGGTATCAATCATTGGGACTAAGTA |
| | | | | | | | TAAAGAGCTGCAAAAATGGATTATTGATATGGGTCGGGAATATGTG |
| | | | | | | | ACTCATTACGCATCCATCTGCAATAAGGTACGTAACCCGGCCGCTT |
| | | | | | | | TATTATCTATTTCCTGCCATTCCTGTTCC |
| STM3070- | 3233025 | 44 | 0.8 | 1.4 | 2.8 | 3.1 | CGTTACGCCCGATGCGACCAAAGCCATTAATCGCTATGCGTACGG |
| STM3071 | | | | | | | TCATAGGTCTCCTGCAAGGCTATCCCGATTCAGATGAGGCTGACAG |
| | | | | | | | AGTAATGCAGCTCATCGTCGAGTAAAACCTCACCTGTCGCAAACTG |
| | | | | | | | CGACTGATTGGTTAATTGTCGAACATTTAATTAACTGAAACGCTTCA |
| | | | | | | | GCTAGAATAAGCGAAACGGGGAATAAAAGGAATGTTTGTCCAGTC |
| | | | | | | | GAAGAAGACAGTTATCTGACCTGCATCACATTTCATGGCCGCTTAC |
| | | | | | | | GCTGCAATTTATTCCATATTTAAGAA |
| STM3106- | 3266543 | 45 | 1.1 | 3.5 | 4.6 | 4.6 | TGATTTTGTTGCTGAATCACCACCGCCAGCGATCGTTCCGCCGGTC |
| STM3107 | | | | | | | GCTAAGATGGTGATATTCGGTAAAGCGAACGCTGCGCCGCTGAAA |
| | | | | | | | CCCATTACCAGAGCAGCTAATGCCGTTTTCCTGAAAAACTCCATGT |
| | | | | | | | TATATCTCCAGTTATGTCAACTGGTCGCATTATCTCTATATTGCAGA |
| | | | | | | | CGAATAATGTGACGCCATACGATTAACCAGCGATATATATCCGACA |
| | | | | | | | GAGAGTATTTTTTAGAGATGGATAACAAAATGCAGGAAAAAACAG |
| | | | | | | | AATAAAAAGGCGCAGATACGATCTGC |
| STM3525- | 3688646 | 55 | 0.8 | 3.8 | 1.8 | 5.6 | ACGCCTCTTCTACAGTGATACATTCAAATTGTTCCATGAATCGCTCT |
| STM3526 | | | | | | | TTCATTATTGCCGGTGAAGCCAATTAAGGCATTTTATCGCCCAGTG |
| | | | | | | | TACGTTGACGGAGTAGCTTAGCGCCATAATGTTATACATATCACTC |
| | | | | | | | TAAAATGTTTTTTCGATGTTACCAATAGCGCGTTTCTTTGCTATTATG |
| | | | | | | | TTCGATAACGAACATTTTTGAACTTTAACGAAAGTGCAAGAGGGCA |
| | | | | | | | GCATGGAAACCAAAGATCTGATCGTGATAGGCGGGGGCATTAACG |
| | | | | | | | GTGCAGGCATCGCGGCTGATGCC |
| STM3880- | 4091492 | 61 | 0.9 | 5.4 | 0.1 | 13.8 | GTATTTGCGTCTGCGTGGCAAGCTGTATTTGTTGTTGCAACGCAAC |
| STM3881 | | | | | | | GCCCTGCGCGCGCCGGATCAGTTCGAGATCCCGCCTAACCGCGTG |
| | | | | | | | ATTGAGTTAGGTACGCAGGTCGAGATTTAACCTCCCATCAACATGC |
| | | | | | | | CGGGGGCCGCGTTGGCTTACCCGGCCTGGCCAATCCGTAGATTCC |
| | | | | | | | CACAAGATAATCGCCTGATTTCCGCTAGCGAAACGTTTCGACGGC |
| | | | | | | | GATCACAATTCTGTTACGTCATGATGGTTTTATGAACACATCCGGG |
| | | | | | | | GTTACACTGCGGCCAGCGAAACGTTTCG |
| STM4289- | 4530650 | 71 | 0.9 | 2 | 8.3 | 10 | CATGTTGGTATCCTCAAAAAGTCAGCGGGGGCAAACGCGCCCAAA |
| STM4290 | | | | | | | AATGGCAGATCGCCGAAAAAGGCCGCAATTATACACAAAATCCTT |
| | | | | | | | AGCGTTGTCGGGACTATTGCCGCTTTTATAAAAGGGTCTGCGCCAC |
| | | | | | | | GCCAGTCAGCAATGGTTTACACTCGAATAACCGCTTTTTTACTGTC |
| | | | | | | | ACCACAGCGCATTAGGGCGTCCTTATTTACACCTTTTGACCGAATT |
| | | | | | | | GACATATATGTGTGAAGTTGATCACATATTTAAACCCTGTTAGGGT |
| | | | | | | | AAAAAGGTCATTAACTGCCCATTCAGG |
| STM4418- | 4661108 | 77 | 0.8 | 3.4 | 8.3 | 6 | CGATCTTATAGCTATTGAGAACTCTCGTTTCACAACCTATGTTTTAA |
| STM4419 | | | | | | | TTTCAAAACGATCAATAATGAAACTTATGTTTTGTTATGGGTATCAC |
| | | | | | | | ATTTCGAATTTCATAATCCTGGCGTTTTTTATCGTTAAGATGCTGCG |
| | | | | | | | TTTTACGCAGTGCTCTCCTCTATCTTGATGAAGTTACTTGATTTTATT |
| | | | | | | | GATTTCGCGACAGTACCTGAACTCAATTTGTCAGGGGCCGTACTTT |
| | | | | | | | TTGTTCTTTCCTGGAACATCTCCATTTCGTGATCTTTTGCATGGAATT |
| | | | | | | | TTTCTTCTAATGAATGCA |
| STM4430- | 4674477 | 78 | 1.3 | 6.1 | 5.6 | 8 | ACTACTGACTGCTTTATTCATTGACATATCCCCTAACAGAAGACGG |
| STM4431 | | | | | | | TGTTATTTTTGCTCATACTAAGGTTTGGTGATTTCATTTTCAATAAAA |
| | | | | | | | ATGGAAATAATGTTTTCATTTATTGTTTGAACAAGATCACAGAAATG |
| | | | | | | | GCATTTCCGGGCAACGGGCATGATCGTTTTTTGTTGTGTTTTTTGTT |
| | | | | | | | TTAATTGATTGATTATAAATGTGTTATTTATTTTAAAATCGCATGGAA |
| | | | | | | | GATAAATTTCATTTTCATGAAAAATACGCCTGAATGTCGAAATTTTT |
| | | | | | | | TAACCGTTTTTTGATCTC |

TABLE 2B
Intergenic regions that induce higher GFP expression in tumor than in spleen

| Arbitrary | Cloned | | | | | | Stable/ |
|---|---|---|---|---|---|---|---|
| clone | promoter | | 5′ gene | | 3′ gene | Anaerobically | unstable |
| number | orientation | 5′ gene | orientation | 3′ gene | orientation | induced | GFP |
| 85 | + | ylaB | − | rpmE2 | + | | Unstable |
| 86 | − | ybaJ | − | acrB | − | | Stable |
| 87 | − | STM0580 | − | STM0581 | + | | Stable |
| 10 | − | pflE | − | moeB | − | Yes | Unstable |
| 11 | − | hcp | − | ybjE | − | Yes | Unstable |
| 16 | − | orf408 | − | ttrA | − | | stable |
| 20 | − | STM1529 | + | STM1530 | + | | Stable |
| 26 | + | dsbB | + | STM1808 | + | | Stable |
| 28 | − | flhB | − | cheZ | − | | Unstable |
| 30 | − | cspB | − | umuC | − | | Stable |
| 31 | − | cbiA | − | pocR | − | | Stable |
| 34 | − | napF | − | eco | + | Yes | Stable |
| 36 | − | menD | − | menF | − | | Stable |
| 44 | − | epd | − | STM3071 | + | | Unstable |
| 45 | − | ansB | − | yggN | − | Yes | Stable |
| 55 | + | glpE | + | glpD | + | | Stable |
| 61 | + | kup | + | rbsD | + | | Stable |
| 71 | − | phnA | − | proP | + | | Unstable |
| 77 | + | STM4418 | − | STM4419 | + | | Stable |
| 78 | + | STM4430 | − | STM4431 | + | | Stable |

TABLE 3A
Regions that induce GFP expression in both tumor and spleen

| | Tumor | Tumor | Tumor | | | | |
|---|---|---|---|---|---|---|---|
| Spleen | (+) | (+)(−)(+) | (+)(−)(+) | Genome | | | |
| lib1 | lib2 | lib3 | lib4 | position | Genes and | 5′ | cloned |

| | | | | | | | | |
|---|---|---|---|---|---|---|---|---|
| Clone | Median of experiment versus | of peak | intergenic | 5′ | | gene | promoter | |
| No. | input library | signal | regions | gene | Function | orient. | orientation | Sequnce |

Sequenced clones:

| | | | | | | | | | | | |
|---|---|---|---|---|---|---|---|---|---|---|---|
| | 9.42 | 2.94 | 1.48 | 15.51 | 711661 | STM0648 | | | | | |
| 89 | 8.22 | 2.05 | 1.04 | 13.69 | 711724 | IR STM0648- | leuS | leucine | − | − | GAAGGATAGGGAAGCATCGACAGGCA |
| | | | | | | STM0649 | | tRNA | | | GTAATACTTCTCTTTGCTCTCGTCTTCG |
| | | | | | | | | synthetase | | | GTCACTTCAAATGTGCGCTTCTCATCC |
| | | | | | | | | | | | CAGTGAAGCTGTACTTTGGATTCTATCT |
| | | | | | | | | | | | CTTCCGGGCGGTATTGCTCTTGCATGG |
| | | | | | | | | | | | CAGCCAGTAGTCCTGTTTTCGATACAG |
| | | | | | | | | | | | CTACAAATGTAGCTTTAGAGGTGGTGT |
| | | | | | | | | | | | TTAGATCCGCATAGCATAGCCCAAACA |
| | | | | | | | | | | | CGCACGTCAAAACAGGGGGTAGAACAT |
| | | | | | | | | | | | TTGTCGCGCCAGGCGTCCGTGAGGAG |
| | | | | | | | | | | | GTGACGCAAAATGCGACACGACTGAG |
| | | | | | | | | | | | GCAAA |
| | 12.24 | 3.63 | 1.58 | 7.43 | 854765 | STM0789 | | | | | |
| 8 | 12.94 | 4.32 | 1.62 | 7.43 | 854776 | IR STM0789- | hutC | histidine | + | + | CAAGAGTGCGCGTGGTTAACTATCAAA |
| | | | | | | STM0790 | | utilization | | | GAGCATGAGCCTTGTCTGCTCATTCGT |
| | | | | | | | | repressor | | | CGTACAACCTGGTCCGCGTCGCGGATT |
| | | | | | | | | | | | GTTTCTCACGCCCGCTTACTTTTCCCC |
| | | | | | | | | | | | GGGTCGCGCTACCGGCTACAGGGACG |
| | | | | | | | | | | | ATTTATCTCCTGAGCGGACTGCTGCCG |
| | | | | | | | | | | | GAAAACGTGATTGCTGACACAATATAA |
| | | | | | | | | | | | CAAAATTGTATCATTTTTGTTAATTCTAT |
| | | | | | | | | | | | TCTTGTGCTTACTTGTATAGACAAGTAT |
| | | | | | | | | | | | ATGTCTGATTCTTATCTGTGGGTCTGC |
| | | | | | | | | | | | GGCGGTGCCTGATAGTGGCGTTTTAGC |
| | | | | | | | | | | | GT |
| | 5.97 | 2.21 | 2.01 | 6.16 | 854930 | STM0790 | | | | | |
| 12 | 3.55 | 2.26 | 1.48 | 6.75 | 1E+06 | IR STM1055- | STM1055 | Gifsy-2 | − | − | GCTGTATTACTTCTGTAAACGCTGCCTA |
| | | | | | | STM1056 | | prophage; | | | AACTATTTTGAATGTGTCTTAACATAAT |
| | | | | | | | | homologue | | | ATACTCGCCGAATAGTAATTTTGTTAAT |
| | | | | | | | | of msgA | | | GTAATTATATACTACAGTGTGGATATTA |
| | | | | | | | | | | | ATACAATTCTTTTGTTGTTAATTATTATT |
| | | | | | | | | | | | TATGAAATTAATTAAAAGTGAATAAGTT |
| | | | | | | | | | | | AGAGGTGTTTGTTGGCCTTAAAATTACA |
| | | | | | | | | | | | TTTGTTGAGGGGGCTTATATGATATGTT |
| | | | | | | | | | | | TTTATTGTATTGTCGCATTTTTCTTAAGC |
| | | | | | | | | | | | TGAATCCGGATTTTGGGGAGGTGGCTA |
| | | | | | | | | | | | AATGTAAATGACGTGGTTTA |
| | 3.37 | 4.00 | 1.33 | 12.90 | 1E+06 | STM1056 | | | | | |
| | 14.51 | 3.69 | 4.70 | 15.31 | 1E+06 | STM1264 | | | | | |
| 14 | 14.95 | 4.14 | 4.70 | 15.31 | 1E+06 | IR STM1264- | aadA | Aminoglycoside | + | + | CAGTTGCCAGAAGATTATGCTGCCACG |
| | | | | | | STM1265 | | adenyltransferase | | | TTGCGTGCGGCGCAGCGTGAATATTTA |
| | | | | | | | | | | | GGTCTGGAGCAACAGGACTGGCATATT |
| | | | | | | | | | | | TTGCTGCCTGCGGTCGTACGCTTTGTG |
| | | | | | | | | | | | GATTTTGCCAAAGCGCACATCCCCACG |
| | | | | | | | | | | | CAGTTCACATAAGATGCCCCAGGACGT |
| | | | | | | | | | | | CTGTCAGGTTGCGCAAACGGCGTTCCT |
| | | | | | | | | | | | CAACTACTACTTAATAGGTTCTCATCGC |
| | | | | | | | | | | | TGAAGTAAGCAGATGATCTTATGCGGG |
| | | | | | | | | | | | CCATCGAATGGATATTCCCACATGGCT |
| | | | | | | | | | | | CTCGTTTTGTTGAGGTGGATATGACTG |
| | | | | | | | | | | | GTT |
| | 14.98 | 5.19 | 4.38 | 12.05 | 1E+06 | STM1265 | | | | | |
| | 6.70 | 7.16 | 4.44 | 21.25 | 2E+06 | STM1481 | | | | | |
| 19 | 8.71 | 5.95 | 5.19 | 17.03 | 2E+06 | IR STM1481- | STM1481 | putative | − | + | TAATGACGATTTTTAGACCATTGAGCGT |
| | | | | | | STM1482 | | membrane | | | GATGATCGGTTTTGCCATATCAGTCCC |
| | | | | | | | | transport | | | TGTTTTCTGATGCCGACACGAATAATAA |
| | | | | | | | | protein | | | TGTGATGTCGGTCGACCTGTTCTGGTT |
| | | | | | | | | | | | AAAATCAAACACTTCAGGTAAAGAAGT |
| | | | | | | | | | | | GAAAATATTTTGAGTTAATTCCTGGCTT |
| | | | | | | | | | | | ATGATACAAATCAGGCGTGTTCAACTA |
| | | | | | | | | | | | CCGAGGACAATTATCATCCGCGATGAC |
| | | | | | | | | | | | GAGAAGCAACACTGCGGATAATTGTAA |
| | | | | | | | | | | | TATTATGGACAATATGTTCAGCGCTTTT |
| | | | | | | | | | | | TTCTCCACGCAAACGCATCTTCACTCT |
| | 6.11 | 3.79 | 0.21 | 11.96 | 2E+06 | STM1686 | | | | | |
| 23 | 5.95 | 3.26 | 0.41 | 14.78 | 2E+06 | IR STM1686- | pspE | phage | − | − | ATTAATCGCGCCCTGAATATGCTCTCG |
| | | | | | | STM1687 | | shock | | | CTGATATTGTTCCGGAATGCGGACATC |
| | | | | | | | | protein | | | TATCCAGTATTCTGCGGCATAAAGCGG |
| | | | | | | | | | | | CATGGCTATGAATAACGCTAACGCAAA |
| | | | | | | | | | | | TATTCCTTTTTTCAACATACTTCCGTCC |
| | | | | | | | | | | | TGACACGTAATGTATTTCGCACACACTA |
| | | | | | | | | | | | TACGCCAGAGCTTAACGAAATATTATGA |
| | | | | | | | | | | | CCAGACTCGCTATTTGTAACGCTGCGA |
| | | | | | | | | | | | AATTTTATTCGCCGCCTTACGAAGTACT |
| | | | | | | | | | | | GGCTCCAGCGCAAACGCCAGCAACATT |
| | | | | | | | | | | | TTTAGCGGACGACGGGCGACGGATTTT |
| | 5.70 | 3.10 | 0.47 | 12.75 | 2E+06 | STM1687 | | | | | |
| | 4.88 | 2.19 | 4.27 | 4.16 | 2E+06 | STM1697 | | | | | |
| 24 | 11.13 | 4.14 | 5.28 | 9.30 | 2E+06 | IR STM1697- | STM1697 | putative | − | − | ATCTTAACTCCCTGATAATGCGCTTTTA |
| | | | | | | STM1698 | | Diguanylate | | | ACGCAAATCAATCAATAAAAACGATCAA |
| | | | | | | | | cyclase/phosphodiesterase | | | TATATAAAAAATGATCGAAAAAACAATA |
| | | | | | | | | domain 2 | | | TATGTTAACTTCATGATAACTTGCTAAT |
| | | | | | | | | | | | TTTATGTTTTGAGAATGTTCTTCTATTG |
| | | | | | | | | | | | CTATAAGGAAATTTACATACTACGCCGA |
| | | | | | | | | | | | ACAACGCTAATACGACGGCATGAGACC |
| | | | | | | | | | | | ATCCGTAAAGCCAGGTTTTTCTTGTCAG |
| | | | | | | | | | | | GCAGAGGGGAAAAATCAAGGCGAGTTA |
| | | | | | | | | | | | ATGTTGTTACACCATTGCGAGGCATTTC |
| | | | | | | | | | | | ACCCACTATGGCAGCGCGGCATC |
| 25 | 11.89 | 5.62 | 3.76 | 13.35 | 2E+06 | IR STM1805- | fadR | negative | − | − | ATGACCATAGTGAGATTTCCATTACACA |
| | | | | | | STM1806 | | regulator | | | GCAAAACATAGTTGCACTCATCATACCA |
| | | | | | | | | for fad | | | GACGGGCGTAACACCTGATAGCGGAC |
| | | | | | | | | regulon | | | GCAATGAAGAAAAAGGGGATCAAGGCA |
| | | | | | | | | and | | | CCATTTCTGATATCGCCTGCCAATATCG |
| | | | | | | | | positive | | | TTAAGGACTTGCTTGCATTCGTCGCGC |
| | | | | | | | | activator of | | | TCGCTACTCTCTGTGTTTAAACATAAAA |
| | | | | | | | | fabA (GntR | | | ACGCTATTTCATTTTTCTAGGTAAGGAA |
| | | | | | | | | family) | | | AAATTTCATGGAGATCTCATGGGGTCG |
| | | | | | | | | | | | CGCCATGTGGCGCAACTTTTTAGGCCA |
| | | | | | | | | | | | GTCGCCCGACTGGTACAAACTGGCACT |
| | 12.08 | 3.58 | 3.13 | 11.54 | 2E+06 | STM1806 | | | | | |
| 27 | 5.39 | 3.93 | 3.96 | 9.39 | 2E+06 | IR STM1838- | yobF | putative | − | + | CTGAAAAGCCATTTTTCTACCATAGCTC |
| | | | | | | STM1839 | | cytoplasmic | | | AATAACTTCGCTTCTTCCAGTGCATCAA |
| | | | | | | | | protein | | | ATCACATTTAAAAGCTGTATTTTTCATAT |
| | | | | | | | | | | | CACTTTTTATGCTGAGTTATGCATAAAT |
| | | | | | | | | | | | TGTCACAATGATAAAAAACACCTTTTAA |
| | | | | | | | | | | | TCAAAATAATAGAAAAGAAAAGCGATTT |
| | | | | | | | | | | | TCGGCACCGCTTTTTGTGATGTTCTGC |
| | | | | | | | | | | | GTCTTTACAGAATGCCTTAAAATAATGA |
| | | | | | | | | | | | ACAAACAATGACAATCCATAAAGAGAG |
| | | | | | | | | | | | AGAAACGTTTCGCTTTTAATAGAGAATG |
| | | | | | | | | | | | AGCGGTATCACAAAAATGCCAT |
| 32 | 10.42 | 8.43 | 4.63 | 14.61 | 2E+06 | IR STM2122- | udk | uridine/cytidine | − | − | AAGGGGGGCGCCGAAACGCCAAACGC |
| | | | | | | STM2123 | | kinase | | | GGCAATTATAGGGATTTCAGCAGCGCG |
| | | | | | | | | | | | ATACCAGTCCGGCGCTATGCCACGGTG |
| | | | | | | | | | | | AATTTGTTGGCGGCGCATTCGACGTCG |
| | | | | | | | | | | | CGACGTAAAAGCGTTCAGTTTTAACGC |
| | | | | | | | | | | | GGGCAGCGGTTTTATCGACCCGTCTGG |
| | | | | | | | | | | | AGGAGGAATACGCCGGGAGCCACAAT |
| | | | | | | | | | | | TTATATTCAGCCAGCGTATAAATCATTA |
| | | | | | | | | | | | CGCGTTTATACTAGCATAATCACAGAGT |
| | | | | | | | | | | | AAACTGACGCGTCCGGTATTCCGCGAC |
| | | | | | | | | | | | GTTACCGGCGATTCGGATAGAGTGGTA |
| | | | | | | | | | | | ATGA |
| | 8.12 | 6.36 | 3.56 | 11.86 | 2E+06 | STM2123 | | | | | |
| | 14.55 | 10.26 | 7.87 | 17.67 | 2E+06 | STM2182 | | | | | |
| 33 | 14.35 | 7.36 | 8.45 | 14.71 | 2E+06 | IR STM2182- | yohK | putative | + | + | GCGCTGTGCCGAGCTGGATTACCAGG |
| | | | | | | STM2183 | | transmembrane | | | AAGGCGCGTTTAGCTCCCTGGCGCTG |
| | | | | | | | | protein | | | GTGATCTGCGGCATTATTACCTCGCTG |
| | | | | | | | | | | | GTAGCGCCCTTTTTGTTTCCGCTCATTC |
| | | | | | | | | | | | TGGCGGTAATGCGCTAACGACGGGAC |
| | | | | | | | | | | | AAAAGACCGGGTTAAAATTTGCGATAC |
| | | | | | | | | | | | GTCGCGCATTTTTCATTGAAGTTTCACA |
| | | | | | | | | | | | AGTTGCATAAGCAATGAGATTTAGATCA |
| | | | | | | | | | | | CATATTAAGACATAGCAGGCCCGTAAA |
| | | | | | | | | | | | CTACGGTTCCATTACATTGTTATGAGGC |
| | | | | | | | | | | | AACGCCATGCATCCACGTTTTCAAACT |
| | | | | | | | | | | | GCT |
| | 11.03 | 8.54 | 7.69 | 12.87 | 2E+06 | STM2183 | | | | | |
| 38 | 14.28 | 2.96 | 0.91 | 8.76 | 3E+06 | IR STM2524- | yfgA | paral | − | − | ATTGCGCAGACGAACGCCGGTGGTTTG |
| | | | | | | STM2525 | | putative | | | TGCTTCATTTTGGTCGTGCGTGGCTTC |
| | | | | | | | | membrane | | | AGTATTCATTCGCTACAGCTACAGGTA |
| | | | | | | | | protein | | | CGTGTAAATTAGGATTCAGGCGCCGAC |
| | | | | | | | | | | | GAGCCGTAATGCCCGCCCACACCGCG |
| | | | | | | | | | | | AAACATCAGGTTAGTTAACCTTAGTCAG |
| | | | | | | | | | | | ACAGTATAAGCCTGTCAGGCCGCAGAT |
| | | | | | | | | | | | GACAAAACCGCTAAGACACAAGGCTAA |
| | | | | | | | | | | | ACTCTTGTTGCACCATTACATACTGCCT |
| | | | | | | | | | | | TAAAGTCGACAAAAACGCACCGTTATTA |
| | | | | | | | | | | | TTGACCAGACAAGTACAACGCCAGACA |
| | | | | | | | | | | | TT |
| | 11.83 | 3.33 | 0.85 | 8.23 | 3E+06 | STM2525 | | | | | |
| | 13.03 | 2.23 | 6.00 | 10.22 | 3E+06 | STM2817 | | | | | |
| 40 | 6.85 | 4.27 | 7.12 | 9.22 | 3E+06 | IR STM2817- | luxS | quorum | − | + | TCCGGCATCACTTCTTTGTTCGGAATG |
| | | | | | | STM2818 | | sensing | | | CAAAAACGCAGATCAAACACGGTGATT |
| | | | | | | | | protein, | | | GCGTCGCCATGCGGGGTGTTCATCGTT |
| | | | | | | | | produces | | | TTTGCAACCCGGACCGCCGGCGCTTG |
| | | | | | | | | autoinducer- | | | CATCCGGGTATGATCGACTGCGAAGCT |
| | | | | | | | | acyl- | | | ATCTAATAATGGCATTTAGTCACCTCCG |
| | | | | | | | | homoserine | | | ATAATTTTTTAAAAATAAACTGAACTCTT |
| | | | | | | | | lactone- | | | TGTTCCGGGGCGAGTCTGAGTATATGA |
| | | | | | | | | signaling | | | AAGACGCGCATTTGTTATCATCATCCCT |
| | | | | | | | | molecules | | | GTTTTCAGCGATGAAATTTTGGCCACTC |
| | | | | | | | | | | | CGTGAGTGGCCTTTTTCTTTTGGGTCA |
| | 9.62 | 3.07 | 4.43 | 3.70 | 3E+06 | STM3279 | | | | | |
| 49 | 9.70 | 3.07 | 4.43 | 4.57 | 3E+06 | IR STM3279- | mtr | HAAAP | − | − | AAAGACCAGCGCCGCCATCGACCAGA |
| | | | | | | STM3280.S | | family, | | | AGAACCACGCCCCGGACATGACCACC |
| | | | | | | | | tryptophan- | | | GGCAGGGAGAACATCCCCGCGCCAAT |
| | | | | | | | | specific | | | GATGGTGCCGCCGATAATCACCACGCC |
| | | | | | | | | transport | | | GCCAAGCAGCGAAGGTGACGTTTGGG |
| | | | | | | | | protein | | | TGGTGGTAAGTGTTGCCATTCAGCTCT |
| | | | | | | | | | | | CTCTCCAGTCATTTATAGTGTGACTATC |
| | | | | | | | | | | | TCTCAATACGCTGCACTGTACCAGTAC |
| | | | | | | | | | | | ACGAGTACAAAAGAAATAAAAAAAGCC |
| | | | | | | | | | | | CCGATTGTGACGATCGGGGCTGTATAT |
| | | | | | | | | | | | TTTACTTTACGCTGTGAATGCGCAGGT |
| | | | | | | | | | | | CAGCGTG |
| | 8.14 | 2.72 | 5.09 | 7.11 | 4E+06 | STM3441 | | | | | |
| 51 | 9.79 | 4.25 | 6.03 | 9.40 | 4E+06 | IR STM3441- | rpsJ | 30S | − | − | TTCCGCGGTTGATTGATCGATCAGACG |
| | | | | | | STM3442 | | ribosomal | | | ATGATCAAACGCTTTCAGGCGGATACG |
| | | | | | | | | subunit | | | GATTCTTTGGTTCTGCATGAGACCAGA |
| | | | | | | | | protein S10 | | | GCTCCAATTATTTTATAAACGAAAATGA |
| | | | | | | | | | | | TTACTCCTCACACCCATTACGATTGATG |
| | | | | | | | | | | | GGAGAGTGTAACCGTTCTTACGTAGCT |
| | | | | | | | | | | | CCCCGATTGGGAGCATTGTTAAATAGC |
| | | | | | | | | | | | CAAATCGGCTATTCGAGGTTCAAATCG |
| | | | | | | | | | | | AACCTGCCGTCAATTACGACAAGCCCG |
| | | | | | | | | | | | CGCATTATACGTAAATCTCAGCCTGAC |
| | | | | | | | | | | | GCAAGTGTCGGATAGAAATTAAGCGCT |
| | | | | | | | | | | | TT |
| | 8.53 | 3.07 | 1.15 | 9.96 | 4E+06 | STM3499 | | | | | |
| 98 | 12.65 | 3.17 | 3.46 | 9.93 | 4E+06 | IR STM3499- | yhgE | putative | − | + | AGCACAAGACGCCCTGCAGCAAACCG |
| | | | | | | STM3500 | | inner | | | GTGAGCAACATCCCCCAGCGAGTAGTA |
| | | | | | | | | membrane | | | TGTGAAAGCGCTACACTTTCCATGTCG |
| | | | | | | | | protein | | | TTATCCAGAATGATGAGAAAGCCGCAT |
| | | | | | | | | | | | TATTGCACCATCTGTTCACCGCCAGGC |
| | | | | | | | | | | | GTCGTCATGCATAATTCAGAAAAAAAC |
| | | | | | | | | | | | GCAGAGAGGTGAATCGATATTGTTAAT |
| | | | | | | | | | | | GTTGGTGTTACGTAACTTTCTTACATGA |
| | | | | | | | | | | | ATGCGATTACAGTCACATTATGTCGGT |
| | | | | | | | | | | | CAAAAACACTTCCTTTTAACGTTTTCAG |
| | | | | | | | | | | | AACATTTTCCACAACAAAAGTAGGTTTC |
| | | | | | | | | | | | CT |
| | 2.45 | 3.73 | 12.35 | 19.22 | 4E+06 | STM3500 | | | | | |
| | 6.69 | 2.72 | 5.18 | 8.20 | 4E+06 | STM3568 | | | | | |
| 57 | 9.77 | 2.89 | 3.26 | 7.29 | 4E+06 | IR STM3568- | rpoH | sigma H | − | − | CCGTCAGCGAGCAACAACCGTGCCAAA |
| | | | | | | STM3569 | | (sigma 32) | | | GCCGATGAGCAACGAGAATATCACCCA |
| | | | | | | | | factor of | | | CTCTTTTATCAGACAGTGATTTTATCCA |
| | | | | | | | | RNA | | | CAAGTTCAATGTAACACTGTGCATAATT |
| | | | | | | | | polymerase; | | | TGCACAAATCTTGTGACATAAAGATGAC |
| | | | | | | | | transcription | | | GCGCGGGGAAGAGACAACAGGGACTC |
| | | | | | | | | of heat | | | TTTCCCTGCGAACGGAAGCCCATTGCA |
| | | | | | | | | shock | | | GGGAAAGATTATACCACGATTTTATCAA |
| | | | | | | | | proteins | | | TCGGGAGTAAAGTGACGTAAATGTTGC |
| | | | | | | | | induced by | | | ACCGTGGCCAGCCAGGCGGCGATCCA |
| | | | | | | | | cytoplasmic | | | GCCAATCATGGAACAGACCAGCAGCAG |
| | | | | | | | | stress | | | CA |
| | 8.29 | 1.81 | 2.41 | 6.08 | 4E+06 | STM3569 | | | | | |
| 58 | 11.88 | 3.48 | 0.80 | 7.56 | 4E+06 | IR STM3621- | yhjR | putative | − | − | TATTTCTCACTGGCAGCATTACGCCCC |
| | | | | | | STM3622 | | cytoplasmic | | | GTCGTCAATACGGGAGAACGCGCATTT |
| | | | | | | | | protein | | | TTCATCTTTCCGTGACATCATTTATAAT |
| | | | | | | | | | | | GTGTAAAAATGCAAAGCGCAGAGTTAC |
| | | | | | | | | | | | AGGGCATCCTGCCGGGCAAATTGATTC |
| | | | | | | | | | | | ACATGCTAAATCTGATGCGTTTTAATTT |
| | | | | | | | | | | | CAATGTTAGGTTTATTTCTGTGCTTTCG |
| | | | | | | | | | | | CTAGTAAACTGATAAACAGTTAAAATAG |
| | | | | | | | | | | | TGACATGAGGGACACTGTGGACCCCGT |
| | | | | | | | | | | | ATTTTCTCTCGGCATCTCATCATTATGG |
| | | | | | | | | | | | GATGAACTGCGCCATATGCCAACCGG |
| | 16.45 | 3.98 | 8.19 | 0.85 | 4E+06 | STM3622 | | | | | |
| 59 | 7.64 | 2.84 | 0.85 | 8.98 | 4E+06 | IR STM3624- | yhjU | putative | + | + | AAACCGCGCCGGTTTCAGAAAACGCTA |
| | | | | | | STM3624A | | inner | | | ATGCGGTGGTGATTCAGTACCAGGGTA |
| | | | | | | | | membrane | | | AGCCCTACGTTCGTCTGAATGGCGGCG |
| | | | | | | | | protein | | | ACTGGGTGCCTTACCCGCAGTAAACCG |
| | | | | | | | | | | | AAAAAGGCCGCAAGGTTTCCCCTGCGG |
| | | | | | | | | | | | CCTGGTTCGGGCGCATGTTGCCATTAC |
| | | | | | | | | | | | GGCGGACAGACGCTCAAAACGCGTTA |
| | | | | | | | | | | | CTTCCTGTCACGTAGCCAGTTGACGAT |
| | | | | | | | | | | | CACACTGGCGATAATGCCAGCAATGAT |
| | | | | | | | | | | | CGGCGCTGCCAGATCGTGCCAGAAGA |
| | | | | | | | | | | | CCACGCCCAACTGCGTAAGCGTCATAT |
| | | | | | | | | | | | AGCCGC |
| 60 | 7.89 | 2.21 | 5.33 | 8.90 | 4E+06 | IR STM3838- | dnaA | DNA | − | − | ATGATTGTTGGCGCACGTCGATAAGA |
| | | | | | | STM3839 | | replication | | | CCCTGCATGAAGGGTGACGCACGAAC |
| | | | | | | | | initiator | | | CGCTGTCTGCGGTTTTCACGGATCTTT |
| | | | | | | | | protein | | | CAAACGATCGCGACTTCACGCAGTCT |
| | | | | | | | | | | | GAAAAATTTCGTGTTCATGCCTGACCA |
| | | | | | | | | | | | GGATCGTTTGAAACGATCAGGACCGC |
| | | | | | | | | | | | GGATCATAGCCTAAACTGAGCAAGAG |
| | | | | | | | | | | | ATCTTCTGTTTCTCACAGATTCTTCCCT |
| | | | | | | | | | | | ATTTATCCACAGGACTTTCCAGGAAAG |
| | | | | | | | | | | | GATAAGTGTAATCGATCCTGGGGAAC |
| | | | | | | | | | | | TCCTGTACGCTTTCGCGCGCATATTGA |
| | | | | | | | | | | | AAAAATTAA |
| | 9.27 | 4.10 | 3.20 | 7.80 | 4E+06 | STM3938 | | | | | |
| 100 | 9.27 | 4.10 | 2.88 | 8.41 | 4E+06 | IR STM3938- | hemC | porphobilinogen | − | + | GTGTGACCATCGGCACCAGTTCTACCG |
| | | | | | | STM3939 | | deaminase | | | TCAGTCCCGGATGGGTTGCCATCAATG |
| | | | | | | | | (hydroxymethylbilane | | | CGTCTTTGACATAATGTGCCTGCCAAA |
| | | | | | | | | synthase) | | | GCGCAAGGGGACTTTGGCGTGTGGCA |
| | | | | | | | | | | | ATTCTTAAAACATTGTCTAACATGCTTG |
| | | | | | | | | | | | TTACCGTCATTATCAATCATTGACCATC |
| | | | | | | | | | | | CTAACATCCTTATAGAGAGTATGTTAGT |
| | | | | | | | | | | | TTTCCGGTCACCGTGAGTGAGAGGATA |
| | | | | | | | | | | | AGGCGCAGTGTCGTCAATGACAGTGAA |
| | | | | | | | | | | | TAATGACGAGAAACCGCCAGCCCGTAT |
| | | | | | | | | | | | TTAAGAATTTACACGCAGCGAACGGTG |
| | | | | | | | | | | | CT |
| | 9.67 | 4.61 | 4.08 | 6.29 | 4E+06 | STM3939 | | | | | |
| 63 | 11.21 | 8.20 | 5.10 | 11.30 | 4E+06 | IR STM3967- | dlhH | putative | − | + | TAACAAACCACATTGCCTTAAAGCGGC |
| | | | | | | STM3968 | | dienelactone | | | TATCTTTTGTGCAATGCCTGGCGATATT |
| | | | | | | | | hydrolase | | | GATTATTTATTGTGATGAACATCACTTT |
| | | | | | | | | family | | | TTAATGGTAAGCGAGTGCAATTGTTTTA |
| | | | | | | | | | | | CGTCATAGTGATGGCTGTCACGAAAAT |
| | | | | | | | | | | | ATCTTTATGCCTTAGGTAAAGTGTCTCT |
| | | | | | | | | | | | TTGCTTCTTCTGACAAACCCGATTCACA |
| | | | | | | | | | | | GAGGAGTTTTATATGTCCAAGTCTGAT |
| | | | | | | | | | | | GTTTTTCATCTCGGCCTCACCAAAAAC |
| | | | | | | | | | | | GATTTACAAGGGGCCCAGCTCGCCATC |
| | | | | | | | | | | | GTCCCTGGCGATCCTGAGCGTGTGGA |
| | 12.98 | 8.20 | 5.93 | 12.83 | 4E+06 | STM3968 | | | | | |
| 66 | 9.91 | 4.92 | 5.25 | 10.47 | 4E+06 | IR STM4087- | glpF | MIP | − | + | TGAATTGAATCATTTCATTAACCAATAT |
| | | | | | | STM4088 | | channel, | | | GTTAACACTTTTAAGTTATTGAATGAAT |
| | | | | | | | | glycerol | | | GTTACCAGGAGATGGATGAAAATTGCT |
| | | | | | | | | diffusion | | | GCAAACCGCGATCTACGCGGTATGTCG |
| | | | | | | | | | | | CTGGACAGCGAGAGCGGGGCTTCATA |
| | | | | | | | | | | | CAATCGACACTATATATTGTGCGCGTTT |
| | | | | | | | | | | | ACGTGAAGCGTCGCCTTGCAATTCAGG |
| | | | | | | | | | | | AGAGGTAAGATCATGTCTTTAGAAGTG |
| | | | | | | | | | | | TTTGAGAAACTGGAAGCAAAAGTACAG |
| | | | | | | | | | | | CAGGCGATTGACACCATCACCCTGTTA |
| | | | | | | | | | | | CAGATGGAAATTGAAGAGCTGAAAGAA |
| | | | | | | | | | | | AA |
| | 9.91 | 3.66 | 4.69 | 10.65 | 4E+06 | STM4088 | | | | | |
| 69 | 8.48 | 1.96 | 2.59 | 6.91 | 4E+06 | IR STM4164- | thiC | 5′- | − | − | CAGCCTTTTCCACTTCATCCTTCGCGCT |
| | | | | | | STM4165 | | phosphoryl- | | | GCCTCTTCGTTGGCTTCGTCCGCTCAC |
| | | | | | | | | 5- | | | TCCAGTCACTTACTTATGTAAGCTCCTG |
| | | | | | | | | aminoimidazole = | | | GAGATTCACCGACTTGCCGCCTTGACG |
| | | | | | | | | 4- | | | CATCACGAACGCTTTTGTGGAAAATTA |
| | | | | | | | | amino-5- | | | GCACTCCGACAAGATAACCGCCCCTCC |
| | | | | | | | | hydroxymethyl- | | | GAAGAGGGGGCTGAAGTAAACTACCC |
| | | | | | | | | 2- | | | GTTACTCGCGCAGAACTCAAGCGGGAC |
| | | | | | | | | methylpyrimidine-P | | | GTTTGACTCTGGCGCCGTCGTGCATCG |
| | | | | | | | | | | | CGTCAAACACCAGCATAATCAGCTTGT |
| | | | | | | | | | | | CTTCCAGCACAAAGCGGGCTTCCAGCG |
| | | | | | | | | | | | CTT |
| | 16.14 | 4.52 | 2.44 | 17.65 | 4E+06 | STM4165 | | | | | |
| | 9.06 | 5.41 | 2.57 | 13.59 | 5E+06 | STM4335 | | | | | |
| 73 | 4.55 | 3.75 | 1.43 | 7.08 | 5E+06 | IR STM4335- | ecnA | putative | + | + | TTCGCGCCTCAATGATGAAACGCTTTAT |
| | | | | | | STM4336 | | entericidin | | | CGGTCTTGTCGCGCTGGTTCTTCTTAC |
| | | | | | | | | A precursor | | | CAGCACATTATTAACGGCATGTAATACC |
| | | | | | | | | | | | GCCCGCGGCTTCGGCGAAGATATTCA |
| | | | | | | | | | | | GCATCTCGGCCACGCCATCTCCCGTGC |
| | | | | | | | | | | | AGCCAGCTAATCGCTTCTCGTCTTCCT |
| | | | | | | | | | | | AAAATTAGTCGATCGCCCATCATTTTCT |
| | | | | | | | | | | | GGGATGTTGTCTATTATTAAGTTGCTAT |
| | | | | | | | | | | | ACACAAACAACATTGGCTAGAAAAGGA |
| | | | | | | | | | | | AGACATTATGGTTAAAAAGACAATTGCA |
| | | | | | | | | | | | GCGATCTTTTCTGTTTTGGTACTTTCC |
| | 3.12 | 2.34 | 0.87 | 3.98 | 5E+06 | STM4336 | | | | | |
| | 10.88 | 3.11 | 4.71 | 12.55 | 5E+06 | STM4399 | | | | | |
| 75 | 17.04 | 4.02 | 5.83 | 15.54 | 5E+06 | IR STM4399- | ytfE | putative | − | − | TTTCCGCCGCAGCAGTAATCCATATCG |
| | | | | | | STM4400 | | cell | | | TACTGGCGAAACAGCGCCGATGCGCG |
| | | | | | | | | morphogenesis | | | GGGAATAGAGAGCGCCAGTTCGCCTAA |
| | | | | | | | | | | | AGGTTGATCGCGATAAGCCATAGCCGT |
| | | | | | | | | | | | TACCTCATTTGCAATAATATAAGTTGTA |
| | | | | | | | | | | | TTTTAAATGCATCTTTAAGGCGAAGCTA |
| | | | | | | | | | | | TAACTCTTTCGGGGTGCGTATAATTTAA |
| | | | | | | | | | | | GCGAGTATGAAATTAGCGTTCCGTGAC |
| | | | | | | | | | | | CGGAACGACGGTCGCTTTTTCCGGTTT |
| | | | | | | | | | | | CGCTCTCACGGCAATGACCACGCCCG |
| | | | | | | | | | | | CCACCAGGAGCGCAATGCCGCTTAAC |
| | | | | | | | | | | | GTCA |
| | 14.72 | 4.99 | 5.83 | 17.37 | 5E+06 | STM4400 | | | | | |
| 76 | 12.10 | 8.37 | 0.91 | 15.76 | 5E+06 | IR STM4405- | ytfJ | putative | − | + | GTGATCCGACCACTTTGGGCCGATAGT |
| | | | | | | STM4406 | | transcriptional | | | TAATCATATGTGCGATTGATGCTTTTTC |
| | | | | | | | | regulator | | | CCGCAAAGGGGATGCCAGTTTGCGGG |
| | | | | | | | | | | | CGGGCGCACACTTCCTGTGAAAAATGA |
| | | | | | | | | | | | AGGCATATACTGAGAAAAATGAGCTGA |
| | | | | | | | | | | | TGTTTAGATAATTCTGAATAACTGTAAT |
| | | | | | | | | | | | CAAAAGGTAAATATACTTATGCACACTG |
| | | | | | | | | | | | GAAACGACGTAGATATGGTCTATAGTC |
| | | | | | | | | | | | ATATGGCATTAAAATTTGCGCCTTAAAA |
| | | | | | | | | | | | CTGTTGGGCCGATTGTGGCATCGCAAG |
| | | | | | | | | | | | GGCGTAATACTCTGCAGGAGACAACAAT |
| | 11.07 | 9.07 | 0.91 | 14.42 | 5E+06 | STM4406 | | | | | |
| | 7.73 | 4.88 | 4.40 | 7.19 | 5E+06 | STM4484 | | | | | |
| 82 | 7.87 | 4.97 | 4.70 | 7.43 | 5E+06 | IR STM4484- | idnD | L-idonate | − | − | GATAATAATGTAAGTCAGACCCACAAAT |
| | | | | | | STM4485 | | 5- | | | GCCGCCACGGGTAATTTGTACGAGAGT |
| | | | | | | | | dehydrogenase | | | TCCTTTATTATTCCATTCAATATTTTGTT |
| | | | | | | | | | | | CCGTAACGGCAACAGCACGCTTACCCG |
| | | | | | | | | | | | CAACAACGCAGGATTGAGTTTTTACTTC |
| | | | | | | | | | | | CATAAATTCCTCACTGGTCAGGTAGTTA |
| | | | | | | | | | | | CCCTGAACGCATTTAAGCGGTTTTATTT |
| | | | | | | | | | | | GTCACTATTTGTGACTTATGTCACGCTG |
| | | | | | | | | | | | GAAAATTGTTACACTACAATGTTACGCA |
| | | | | | | | | | | | TAACGTGATGTGCCTTAGAGTTCTTCTC |
| | | | | | | | | | | | TATGGAAATTAAAAAACGTGAA |
| | 4.40 | 3.55 | 6.66 | 4.67 | 5E+06 | STM4485 | | | | | |
| 102 | 6.83 | 4.51 | 1.52 | 4.48 | 5E+06 | IR STM4551- | STM4551 | putative | − | − | ATACACGGAATCGGGCGCCAACATGAA |
| | | | | | | STM4552 | | diguanylate | | | AATAACGTATGAGAAAAGGTCGCCTAA |
| | | | | | | | | cyclase/phosphodiesterase | | | AGCGAGGTGTTGTTGTTTTTACGTTAAC |
| | | | | | | | | domain 1 | | | AGTCGGACAATTTATCACCTTACTGAAT |
| | | | | | | | | | | | ACGTGTCATCAACCGTTAAGTAAAACTC |
| | | | | | | | | | | | ATCTCTTTAGCTTTCTCCCTGGCTGACA |
| | | | | | | | | | | | AATGAGAAAATATATCATATGATATTGG |
| | | | | | | | | | | | TTATCATTATCAATTCCAGAGGTGAAAC |
| | | | | | | | | | | | CATGTTGCAGCGGACGTTAGGCAGCG |
| | | | | | | | | | | | GATGGGGCGTATTATTGCCTGGAGTGA |
| | | | | | | | | | | | TTATCGTTGGACTGGCGTTTATCGGC |
| | 8.88 | 3.83 | 1.44 | 4.96 | 5E+06 | STM4552 | | | | | |
| | 5.54 | 5.79 | 4.40 | 14.79 | 5E+06 | STM4566 | | | | | |
| 83 | 10.24 | 5.19 | 8.33 | 14.49 | 5E+06 | IR STM4566- | yjjI | putative | − | + | CGCTGCTGGAGCGCAGTTTCGCATGA |
| | | | | | | STM4567 | | cytoplasmic | | | GGCAGGCATCTTCGTTTCCTCTTTATG |
| | | | | | | | | protein | | | CCGGGACGATGCGCTATTGTAGAAAAT |
| | | | | | | | | | | | GGCGGCAAACCGACTTTGATCCTGATG |
| | | | | | | | | | | | CGCTTATCGCTCGAAGAACAGACGGTG |
| | | | | | | | | | | | ACGGCGGGATAATTTGATTCAGATCTC |
| | | | | | | | | | | | ATTACAGTAATGCAAATTTGTACGTAGT |
| | | | | | | | | | | | TTTCATTAACTGTGATGTATATCGAAGT |
| | | | | | | | | | | | GTAATCGCGAGTGAATGTTAGAATATTA |
| | | | | | | | | | | | ACAGACTCGCAAGGTGAAATTTTATAC |
| | | | | | | | | | | | GGCAATGCCGTTGGAGAATGTCATGAC |
| | | | | | | | | | | | TG |
| | 8.07 | 5.72 | 5.32 | 11.30 | 5E+06 | STM4567 | | | | | |

Supported by array data only:

| | | | | | | | | | |
|---|---|---|---|---|---|---|---|---|---|
| 7.53 | 3.93 | 3.12 | 16.10 | 39114 | PSLT047 | | | | |
| 6.23 | 9.42 | 4.09 | 21.40 | 39436 | IR PSLT047- | PSLT047 | putative | − | TTCTACCGGATGGTTGAGCACGTTCAT |
| | | | | | PSLT048 | | cytoplasmic | | TTCATAAAATGATGCAAATTCGCCCCTG |
| | | | | | | | protein | | TCAAACACGGCGCCGAAATCGGCTACC |
| | | | | | | | | | GCTTTCCACACTTCGCCGCGATCGACA |
| | | | | | | | | | TTGACAAAGCCTTTATTCCAGTCGCCAT |
| | | | | | | | | | ATCCGAAGCTAAGTTTACCGTATACGC |
| | | | | | | | | | GTTTCAATTCCGCTGCCTGGCCATTAA |
| | | | | | | | | | AGCAAGAGAAAAGAACACATGCGGCGA |
| | | | | | | | | | GTAGACTATTAATATATTTCTTATTTTTC |
| | | | | | | | | | ATGCTCAACTCCATGAGGTAAAAACAC |
| | | | | | | | | | AGTGAAATGTTGTGTAAAGAAGCGAAT |
| 4.20 | 5.90 | 3.12 | 12.13 | 108368 | IR STM0093- | imp | Organic | − | GGTCACAGCCTAACTTACTCATCTTCG |
| | | | | | STM0094 | | solvent | | CTGCGCCAGTGTTAATCCTGCCGTTTA |
| | | | | | | | tolerance | | GCGTCTGTGGTGTTAGGCACGGCATTG |
| | | | | | | | protein | | AATGACAGGTATGATAATGCAAATTATA |
| | | | | | | | | | GGCGATGTCCCACAATTGACCGTAGCC |
| | | | | | | | | | TTCATTTGCAGAAAAGCACCTTATTTTG |
| | | | | | | | | | TGGGAGATAGCCTCACCGATAGCGTAA |
| | | | | | | | | | CGTTTTGGGGAGTCTATGCAGTACTGG |
| | | | | | | | | | GGAAAGATAATTGGCGTCGCCGTAGCC |
| | | | | | | | | | CTGATGATGGGCGGCGGCTTTTGGGG |
| | | | | | | | | | CGTGGTCCTGGGTCTGCTGGTGGGCC |
| | | | | | | | | | ATAT |
| 7.78 | 6.97 | 5.53 | 15.14 | 108588 | STM0094 | | | | |
| 16.16 | 4.53 | 1.45 | 6.75 | 230588 | IR STM0194- | fhuB | ABC | + | TAAATAAAAAACGCTTGTCTTTGGGTTT |
| | | | | | STM0195 | | superfamily | | TTAATGGAAAATACTTCACCGCGCCTAA |
| | | | | | | | (membrane), | | GGGATGTTATTTATTAACGTGTTGTTTG |
| | | | | | | | hydroxamate- | | CTTCTTTTGAATGTTGCATCGGCAATTT |
| | | | | | | | dependent | | CATAACTCGTCATATAATATATATCTAC |
| | | | | | | | iron uptake | | TAATATAAACATGGGGTATTGAGTATAA |
| | | | | | | | | | CTCTGTGTGAATAGCGTAAAAATACTCA |
| | | | | | | | | | CCAACTTTTAATAAGGATGAAAAATGAA |
| | | | | | | | | | TACAGCAGTAAAAGCTGCGGTTGCTGC |
| | | | | | | | | | CGCACTGGTTATGGGTGTTTCCAGCTT |
| | | | | | | | | | TGCCAATGCTGCGGGCAGTAATA |
| 16.16 | 4.05 | 1.60 | 7.30 | 230618 | STM0195 | | | | |
| 5.06 | 3.61 | 3.18 | 11.78 | 256949 | STM0218 | | | | |
| 5.06 | 3.81 | 3.87 | 10.76 | 257001 | IR STM0218- | pyrH | uridylate | + | GCTGGATAAAGAGCTGAAAGTGATGGA |
| | | | | | STM0219 | | kinase | | TCTGGCGGCGTTCACGCTGGCTCGTG |
| | | | | | | | | | ACCACAAACTGCCGATTCGTGTTTTCAA |
| | | | | | | | | | CATGAACAAACCGGGCGCGCTGCGTC |
| | | | | | | | | | GTGTGGTGATGGGCGAAAAAGAAGGG |
| | | | | | | | | | ACGTTAATCACGGAATAATTCCCGTGA |
| | | | | | | | | | GCGCCAAATACGGGTAAGATTCTGTTC |
| | | | | | | | | | TATTGACGGGTCTTATTACCTGGCAGA |
| | | | | | | | | | AATTAAACGAGACTATACTTAGCACATC |
| | | | | | | | | | TTTATATTGTGTGACCGTCTGGTCTGAC |
| | | | | | | | | | TGAGACTAGTTTTCAAGGATTCGTAAC |
| | | | | | | | | | GTGA |
| 13.58 | 3.14 | 2.83 | 10.90 | 258882 | STM0220 | | | | |
| 9.50 | 3.85 | 3.09 | 6.86 | 259045 | IR STM0220- | dxr | 1-deoxy-D- | + | GATTCGTTTTACCGATATCGCCGGGCT |
| | | | | | STM0221 | | xylulose 5- | | CAATTTAGCGGTGCTGGAGAGGATGGA |
| | | | | | | | phosphate | | TTTACAGGAACCGGCAAGCGTTGAGGA |
| | | | | | | | reductoisomerase | | CGTATTGCAGGTTGACGCCATCGCGCG |
| | | | | | | | | | TGAAGTAGCCAGAAAACAAGTGATACG |
| | | | | | | | | | GCTCTCACGCTGACGATTATCCCGCGA |
| | | | | | | | | | CAGAAGATCGTGCTATTTGTTAGCGTT |
| | | | | | | | | | GGGCTTCGGTGATATAGTCTGCGCCAC |
| | | | | | | | | | CTGATCGCAGGTTTTTGGCTTTTTTCGG |
| | | | | | | | | | TCAGGTTAGCCGTGGTTTTACACGGCT |
| | | | | | | | | | TTTTTGTGGATACACAAAATCATTCAGG |
| | | | | | | | | | AC |
| 9.06 | 3.02 | 0.27 | 4.57 | 280369 | STM0238 | | | | |
| 9.81 | 4.01 | 0.73 | 7.77 | 280632 | IR STM0238- | yaeP | putative | − | AATATTTTTCCACATGCCCTCCTGTCAG |
| | | | | | STM0239 | | cytoplasmic | | CATTCTGACTTAACCGTGGATGCAAGT |
| | | | | | | | protein | | CTAAGCCTACGAAGTTAAATCTTGTTTA |
| | | | | | | | | | GCAAGGTGACTATACCATACTCATTTG |
| | | | | | | | | | CGCAATATCAGCGCCTGACGCGAGTG |
| | | | | | | | | | GGTAAAAGATTCGTTAACAGCCTTTTAG |
| | | | | | | | | | CGCGGTTTTCGCTACAATGGGCGCCTG |
| | | | | | | | | | ATTCGAAAGGAGTTTTCTCATGGCGCT |
| | | | | | | | | | TAAAGCGACAATTTATAAAGCCGTCGT |
| | | | | | | | | | CAATGTGGCTGACCTTGATCGCAACCG |
| | | | | | | | | | GTTTCTGGATGCGGCATTGACGCTGGC |
| | | | | | | | | | GC |
| 9.19 | 4.19 | 0.72 | 7.77 | 280644 | STM0239 | | | | |
| 21.74 | 9.05 | 6.68 | 14.14 | 350300 | STM0306 | | | | |
| 23.71 | 2.23 | 3.60 | 6.98 | 350713 | IR STM0306- | STM0306 | homologue | − | GACCAGGCTACCACAAGGGGAATGAT |
| | | | | | STM0307 | | of sapA | | GCAGACTGCGAAAAAGTTTTTCATTTCA |
| | | | | | | | | | GAACCTGCCTTAATATTGGGCTAAAAG |
| | | | | | | | | | ACAAGTTTCACGGTATAGGGTGTGATA |
| | | | | | | | | | TAACGATTACATAAACGAAGCCCAAAAA |
| | | | | | | | | | ACGGTCTATTGTAACGCTGGGTTTTCT |
| | | | | | | | | | GTAAGCGGGTAAAAAATGAGATGAAGA |
| | | | | | | | | | TTTTAAATAACAATACGATAATCGTCGG |
| | | | | | | | | | TATGGAAATCCATCTCCTCGCCAAATTG |
| | | | | | | | | | CCCCACGTACGGTTTCACTTCTACGTT |
| | | | | | | | | | ATGTAACGGGTAGTGTGAGATGGAGCGA |
| 18.23 | 3.38 | 2.66 | 8.07 | 350910 | STM0307 | | | | |
| 4.50 | 3.64 | 1.20 | 6.94 | 385496 | IR STM0340- | stbA | putative | − | AAACAGTATAATTAGTCTTACTTTTTTCT |
| | | | | | STM0341 | | fimbriae; | | TACTTTTGGCCTTTCAGAAGTTTCCTGA |
| | | | | | | | major | | GTTTGCGTTAAGGTAAAGAAAAGTGTT |
| | | | | | | | subunit | | CAGATTTACCTATAACTGTTTGATTTGT |
| | | | | | | | | | AATGTGTAGGTAATACTTGTGTCAATTA |
| | | | | | | | | | TTGTTTACTATAAGTGAGACTTATAAGT |
| | | | | | | | | | TAAACTCAGGTTAATTAGGGGGCTGAA |
| | | | | | | | | | TTCTTTTTTGAGCATGATAATATGTCGT |
| | | | | | | | | | CTGAATGATGGATGCAGTTACCTTTAG |
| | | | | | | | | | GATTGTCATGAATGAAACTATATTTTTA |
| | | | | | | | | | CTTGATAAGCGTGTTGTATTTGA |
| 4.42 | 3.55 | 1.12 | 6.31 | 385529 | STM0341 | | | | |
| 6.92 | 7.96 | 4.23 | 12.59 | 386588 | STM0342 | | | | |
| 7.27 | 7.41 | 4.09 | 11.40 | 386656 | IR STM0342- | STM0342 | putative | + | AATCCGGCAGGATTACCCTACACTACG |
| | | | | | STM0343 | | periplasmic | | ATGTTACTACCGATACGAAAGAGAAAC |
| | | | | | | | protein | | GGCTTTTTTTCGTGATATCTGCATCAGC |
| | | | | | | | | | AAACTGCGCAGAACGGGTATGAAAACA |
| | | | | | | | | | TTTACTTTTAAAGTCAATTCAGTTAAGA |
| | | | | | | | | | CTTTTGAGTCTGATACTGCTGGCGATTT |
| | | | | | | | | | GTTTTCCTGGTTGAGACTGTTACAGCC |
| | | | | | | | | | TGGTACGATTAATGAGTTAAAGATGGT |
| | | | | | | | | | CAAAATTGGGAAAAATACCTACATGTTT |
| | | | | | | | | | TCGCTTAATCGACATTTGTATAATGTGT |
| | | | | | | | | | GTACCACCAGTAGTAACGTTGAGTTG |
| 2.14 | 2.18 | 0.75 | 4.10 | 450515 | STM0396 | | | | |
| 8.70 | 2.17 | 1.65 | 3.75 | 450651 | IR STM0396- | sbcD | ATP- | − | AAAGCCTGATGCTCCGCGGCGCGGCT |
| | | | | | STM0397 | | dependent | | TTTACTGTAGAAATTTTGTCCCAGATGC |
| | | | | | | | dsDNA | | CAGTCAGAGGTGTGGAGGATGCGCAT |
| | | | | | | | exonuclease | | AATTGTTCCATGCAAAAAAAGCGTGAA |
| | | | | | | | | | CGGGATTATACACGTCATCCCTTCCATT |
| | | | | | | | | | TTTGGGCGCAATTTACCGCCGGTACAC |
| | | | | | | | | | GGTAATGCATGGTTTCACCGGTGTCAT |
| | | | | | | | | | AAATCATCAACATGCTGTCAATGCCGC |
| | | | | | | | | | CTTTTTTTTTCATAAATCTGTCATAAATC |
| | | | | | | | | | TGACGCATAATGGCGCGGCATTGATAA |
| | | | | | | | | | CTAACGACTAACAGGGCAAATTATGGC |
| | | | | | | | | | GA |
| 12.04 | 5.51 | 3.16 | 0.46 | 450902 | STM0397 | | | | |
| 11.06 | 4.11 | 2.66 | 12.37 | 508340 | STM0451 | | | | |
| 11.06 | 4.38 | 2.82 | 12.37 | 508386 | IR STM0451- | hupB | DNA- | + | GGTAGGCTTTGGTACTTTTGCTGTTAAA |
| | | | | | STM0452 | | binding | | GAGCGTGCTGCCCGTACTGGTCGCAA |
| | | | | | | | protein HU- | | CCCGCAAACAGGTAAAGAGATCACCAT |
| | | | | | | | beta, NS1 | | CGCCGCTGCCAAAGTGCCGAGTTTCC |
| | | | | | | | (HU-1) | | GTGCAGGTAAAGCGCTGAAAGACGCG |
| | | | | | | | | | GTAAACTAAGCGTGATCCCCTCGGGGG |
| | | | | | | | | | ATGTGACAAAGTACAAGGGCGCATCAA |
| | | | | | | | | | CTGATGTGCCTTTTTTATTGGCGATTCG |
| | | | | | | | | | GGACTTTCTGTGCGTTGCGGGCTGACA |
| | | | | | | | | | ATTGCCCTCGTTTCTTGTCACAATAGGC |
| | | | | | | | | | TTTTGTGCGCCGCGTTCAGAAAATGCG |
| | | | | | | | | | ATGC |
| 7.10 | 8.00 | 0.37 | 10.82 | 522980 | STM0464 | | | | |
| 5.77 | 4.81 | 0.36 | 9.15 | 523177 | IR STM0464- | tesB | acyl-CoA | − | CTGACCGCCAAATACCTGGCGCAGCC |
| | | | | | STM0465 | | thioesterase | | CTAAGTCTTCACTTTGGCCCCGAAAGA |
| | | | | | | | II | | GTCCTTCTTCAATTTTTTCCAGATTCAA |
| | | | | | | | | | TAATGTCAGCAAATTATTCAGTGTCTGA |
| | | | | | | | | | CTCATACATACTCTCCAGGTGACAACG |
| | | | | | | | | | ATGCCGAAGCGAGGTAGGGCAGAGTA |
| | | | | | | | | | TAACGCAATTTTGCAAGTGGTCCGATG |
| | | | | | | | | | GGTACAAAAGTCTGAATAACAGACCAA |
| | | | | | | | | | TTCCAGGCAAAAATGAGTGACATGTGC |
| | | | | | | | | | CACACTTAATCACGTTATGTTTCTGTTA |
| | | | | | | | | | ACCACTCTTCCGGCGGGGGGAAAGGC |
| | | | | | | | | | CTGC |
| 5.75 | 6.67 | 6.06 | 9.71 | 533588 | STM0476 | | | | |
| 6.79 | 6.13 | 6.93 | 8.40 | 533647 | IR STM0476- | acrA | acridine | − | TCTGGCATCTGCTGGCCGCCTTGCTGG |
| | | | | | STM0477 | | efflux pump | | TCCTGTTTGTCGTCACATCCTGTTAGC |
| | | | | | | | | | GCTAAGCTGCCTGAGAGCATCAGAACG |
| | | | | | | | | | ACCGCCAGAGGCGTTAACCCTCTGTTT |
| | | | | | | | | | TTGTTCATATGTAAACCTCGAGTGTCCG |
| | | | | | | | | | ATTTCAAATTGGTCAATGGTCAAAGGTC |
| | | | | | | | | | CTTAAACCCATTGCTGCGTTTATATTAT |
| | | | | | | | | | CGTCGTGCTATGGTACATACATCCATA |
| | | | | | | | | | AATGTATGTAAATCTAACGCCTGTAAAT |
| | | | | | | | | | TCACCGACATATGGCACGAAAAACCAA |
| | | | | | | | | | ACAACAAGCGCTGGAGACACGACAACA |
| 7.34 | 5.05 | 4.44 | 12.10 | 534374 | STM0477 | | | | |
| 7.30 | 6.03 | 4.23 | 13.57 | 534417 | IR STM0477- | acrR | acrAB | + | TCAGGGCTCATGGAAAACTGGTTATTT |
| | | | | | STM0478 | | operon | | GCTCCGCAATCGTTTGATTTAAAAAAAG |
| | | | | | | | repressor | | AAGCTCGCGCCTACGTCACGATCCTGC |
| | | | | | | | (TetR/AcrR | | TGGAGATGTATCAATTGTGTCCGACGC |
| | | | | | | | family) | | TGCGCGCGTCGACGGTCAACGGCTCC |
| | | | | | | | | | CCCTGATAATATTCCAGGAAAACTCCT |
| | | | | | | | | | GGACATTTTCTGTGTCGCTATTCTGTTT |
| | | | | | | | | | GTTACAGGCGTGATATTCTTGCGACTC |
| | | | | | | | | | AATTATTTCCGGTCTGCTTGCCGGTTCA |
| | | | | | | | | | GACACTTCATTCTCATGACTATGTTGCA |
| | | | | | | | | | GCTTTATAAACGTTCACAGCATTTTGTT |
| 5.99 | 5.29 | 3.53 | 12.94 | 534476 | STM0478 | | | | |
| 2.86 | 2.34 | 0.61 | 8.04 | 598959 | STM0536 | | | | |
| 3.16 | 3.01 | 0.64 | 10.18 | 598994 | IR STM0536- | ppiB | peptidyl- | − | ATGGTGTTGTTGTAAAAACCTTCGCGG |
| | | | | | STM0537 | | prolyl cis- | | CAGTAGTCCAGGAAGTTTTTAACTGTTT |
| | | | | | | | trans | | CAGGCGCTTTATCATCAAAGGTTTTGAT |
| | | | | | | | isomerase B | | TACGATATCGCCGTGATTAGTGTGGAA |
| | | | | | | | (rotamase | | AGTAACCATTTTTGCATCCTGTTCCAAG |
| | | | | | | | B) | | AGAGTGGTGCTTTAGCCCGCAATGGG |
| | | | | | | | | | GCACATATAGGGGCTTGTTATAGCATA |
| | | | | | | | | | ACCGTAAGCTGCGATCACCTTGCAAAG |
| | | | | | | | | | TGTGCTGCTTCGATTACGAATAATATGT |
| | | | | | | | | | ATCATACGGAGATTATTACCCACACAC |
| | | | | | | | | | GTCTATACGGAATCTTCGATGTTAAAAA |
| 2.62 | 2.98 | 0.54 | 7.94 | 599106 | STM0537 | | | | |
| 6.23 | 2.91 | 0.44 | 8.74 | 649485 | IR STM0588- | entF | enterobactin | + | ATTAATAAATAACGGGCGTTGTTTCTGC |
| | | | | | STM0589 | | synthetase, | | CTTTAACAAATTAAATCCTGAAACCCAT |
| | | | | | | | component F | | AATAATTACTAATTATTATGGGTTTTTTA |
| | | | | | | | (nonribosomal | | TTGCAACTATTAATTCTTTTAACATAAGT |
| | | | | | | | peptide | | GATACATGCTACAGGCAAGTTTAATTCC |
| | | | | | | | synthetase) | | GAATATTTAGCTTTTCGGGCACTGGCG |
| | | | | | | | | | CGTAAAGATTGTTTCGGATAATTCTGAC |
| | | | | | | | | | TTGCTGTTAGAATCTCTGACAGGAATGT |
| | | | | | | | | | GTTCTTTCATTGGATAAAGTTTTCAGGT |
| | | | | | | | | | CATACGGCATGCCATCTCTTAATGTAAA |
| | | | | | | | | | ACAAGAAAAAAATCAGTCAT |
| 5.62 | 2.58 | 0.36 | 7.48 | 649550 | STM0589 | | | | |
| 8.75 | 5.12 | 3.69 | 15.76 | 704993 | IR STM0642- | ybeB | putative | − | ACGCCGTGTAGTATACCTGAATCAGCG |
| | | | | | STM0643 | | ACR, | | GCGATACCGGGACTTATGTCGCCGGAT |
| | | | | | | | homolog of | | CGGCGTTTAAAACCAGATTATCATCCC |
| | | | | | | | plant lojap | | ATCCCACGTCACAGAAAGCATCGCCAT |
| | | | | | | | protein | | TTTTGTAAAACAATTTCTGCAAAGCTCT |
| | | | | | | | | | GCAAGGTGAAAAAAGCCTGGCTGCGG |
| | | | | | | | | | AGAATAACAGCCTGTCGGGGGCTGTCA |
| | | | | | | | | | ATGGGCGAAACCGCTGCGGCGAGAAA |
| | | | | | | | | | AAACGGAAAATTCATCACTCAGGCCGC |
| | | | | | | | | | CAGACGGCACGACTATTTAATACTTTCA |
| | | | | | | | | | GGGTGGCGAACCCTTCGCATATGTCGA |
| | | | | | | | | | TTGC |
| 9.05 | 6.18 | 3.69 | 17.29 | 705024 | STM0643 | | | | |
| 11.63 | 6.24 | 8.80 | 8.43 | 766043 | IR STM0701- | speF | ornithine | − | CAATAGACCTGAATGACATAAGGGTCG |
| | | | | | STM0702 | | decarboxylase | | GAAAGACCTGTATGCTGAAGTACCCGT |
| | | | | | | | isozyme, | | AGCAGAAAAACTACCGGGCATTAAAGA |
| | | | | | | | inducible | | AATGAAAGTCGAAACTATTGCGGTGGG |
| | | | | | | | | | CAAACATCATAATATGCGTTGTCCGCCT |
| | | | | | | | | | TATATGGGGCATAAAACGATTATTATTT |
| | | | | | | | | | TCCATTTTGAGGTCCTTTCATTGATTTA |
| | | | | | | | | | TTGAAAGCATGGATATTTTATCCAGGAA |
| | | | | | | | | | GCGCCAGCAATCTGTGAACCAGATCAA |
| | | | | | | | | | CAAAAAACGATCATTTGAAAAATAATTA |
| | | | | | | | | | GTCGGCGATTATGCATATCGTGCTGT |
| 17.22 | 6.49 | 7.28 | 11.13 | 826178 | STM0762 | | | | |
| 12.09 | 3.34 | 5.14 | 8.39 | 826326 | IR STM0762- | STM0762 | fumarate | − | TAATGGTTTCCTTGCCGATCTTTGACTC |
| | | | | | STM0763 | | hydratase, | | TTCTTTATCATATGCTTTACGAAAAGAA |
| | | | | | | | alpha | | CACATGAGATTATCATCCAGTTCATAAC |
| | | | | | | | subunit | | AAGCTTTTTTTACAAGTTTTTCGATAATC |
| | | | | | | | | | GGAATGATAATTTCTGTATTTAATATAC |
| | | | | | | | | | GACTCATACTCCCTCCAGTGCTATGTT |
| | | | | | | | | | GCATTGTTTTATCCATTGATCACATTTT |
| | | | | | | | | | CATGATATTCGTATTCATTGTAGGAAGG |
| | | | | | | | | | AAATATGTTATTTTTATTAAATGATAAAT |
| | | | | | | | | | TTTATTTATAGTAGTGGAAAATAGATGG |
| | | | | | | | | | AAATTAGACAATTAGAATAT |
| 2.29 | 5.25 | 4.55 | 10.15 | 901671 | STM0834 | | | | |
| 7.34 | 4.71 | 0.34 | 5.13 | 902051 | IR STM0834- | ybiP | putative | − | AATGGGCGCCATTTCCGTTGAGGATGC |
| | | | | | STM0835 | | Integral | | AAAATAAAGCGGCGTACCGCACCGCC |
| | | | | | | | membrane | | GCGTTATTTCGTGGAAGGGTTATCCTG |
| | | | | | | | protein | | CTCCGGTTTGCCGTTGATCATATCGCA |
| | | | | | | | | | CAACATAGAGAGCAGCATTAACCGGAC |
| | | | | | | | | | TTTAAAGGGAGAGTGACTGAACACGCG |
| | | | | | | | | | TATACACCTCTTAAATTCGTTCATATAA |
| | | | | | | | | | ACCTCCTGATGTTTCTATCCCATCGATC |
| | | | | | | | | | CGTGAGGGATGTCTGCATTACATACAG |
| | | | | | | | | | ATATAGCACAGGCTATGTTTTATAGCTA |
| | | | | | | | | | TTGCTAAAACGTTAATTTTTTGTGCCCAG |
| | | | | 902276 | STM0835 | | | | |
| 14.20 | 5.38 | 2.63 | 8.80 | 932960 | IR STM0859- | STM0859 | putative | − | CTACCAGATGCGGCAGACATGTAAGTT |
| | | | | | STM0860 | | transcriptional | | TTTTCCGCTCCACGTGTTATGCTCCCTT |
| | | | | | | | regulator, | | CTTCACTGATAGCAAGGAATAATTTTAA |
| | | | | | | | LysR family | | ATCTTTTATATCAAAGTGCATCGTTGTG |
| | | | | | | | | | GCTCATAATTAACGTATAATACAGTGTG |
| | | | | | | | | | CTGCTTTTTTATAGACTCAGTCAGACTG |
| | | | | | | | | | AGTATTTCGGCCTATCCGAATTCCTGTC |
| | | | | | | | | | ACGTCGAGATAACTACAAAATGTAGGC |
| | | | | | | | | | TGACGGTGTCACCGCCCTACCATGATC |
| | | | | | | | | | CGGGGCGGATCTGGTAGGACGCTGGT |
| | | | | | | | | | GACCGCTGACAGGGGGTCAGGTCAGA |
| 13.76 | 7.84 | 2.74 | 10.87 | 933137 | STM0860 | | | | |
| 5.18 | 4.54 | 0.74 | 9.72 | 1E+06 | STM0943 | | | | |
| 8.61 | 7.82 | 1.91 | 22.11 | 1E+06 | IR STM0943- | cspD | similar to | − | TCAGGCGAGGCGTCAAGCATCAGGCA |
| | | | | | STM0944 | | CspA but | | GGGGGGATCGGGTAAAAATGAATCAAA |
| | | | | | | | not cold | | AATTTGAAGCAGTTAACGCTATTGCCG |
| | | | | | | | shock | | GGAATGTGACAGATGTCGCGGATGGTA |
| | | | | | | | induced | | CTGATAGATGTTAGTTATCTATCAATTG |
| | | | | | | | | | AGGTAGATTGATTGTGTGCATAGACTC |
| | | | | | | | | | TGGTCAGCGGCAGATTTTCCTGCCGAC |
| | | | | | | | | | AACTGTAACCGATAATGACGACTGACA |
| | | | | | | | | | ATGGGTAAGACGAACGATTGGCTGGAT |
| | | | | | | | | | TTTGACCAGTTGGTGGAAGATAGCGTG |
| | | | | | | | | | CGCGACGCGCTAAAACCGCCATCTATG |
| | | | | | | | | | TATA |
| 8.61 | 3.76 | 1.91 | 21.37 | 1E+06 | STM0944 | | | | |
| 3.93 | 4.39 | 1.02 | 11.82 | 1E+06 | STM0946 | | | | |
| 2.43 | 3.12 | 0.93 | 4.12 | 1E+06 | IR STM0946- | tnpA_1 | IS200 | + | TATCTGAAGGGTAAAAGTAGTCTGATG |
| | | | | | STM0947 | | transposase | | CTTTACGAGCAGTTTGGGGATCTAAAA |
| | | | | | | | | | TTCAAATACAGGAACAGGGAGTTCTGG |
| | | | | | | | | | TGCAGAGGGTACTATGTCGATACGGTG |
| | | | | | | | | | GGTAAGAACACGGCGAAGATACAGGA |
| | | | | | | | | | CTACATAAAGCACCAGCTTGAAGAGGA |
| | | | | | | | | | TAAAATGGGTGAGCAATTATCGATCCC |
| | | | | | | | | | GTATCCGGGCAGCCCGTTTACGGGCC |
| | | | | | | | | | GTAAGTAACGAAGTTTGATGCAAATGT |
| | | | | | | | | | CAGATCGTATGCGCCTGTTAGGGCGC |
| | | | | | | | | | GGCTGGTAAGAGAGCCTTATAGGCGCA |
| | | | | | | | | | TCTGAAA |
| 4.71 | 5.27 | 1.14 | 8.16 | 1E+06 | IR STM0958- | trxB | thioredoxin | − | TGTAGGGAATTTACAGACGTAAAAAAA |
| | | | | | STM0959 | | reductase | | GAGCATAACGATTTTGTTAACAATATGT |
| | | | | | | | | | GTAATAGCATGAACCGATGAACGGCCG |
| | | | | | | | | | CGACAGCGACGTTATCATCACAAACTT |
| | | | | | | | | | TAATTAAAATCGGTAACTTATAAGGTGA |
| | | | | | | | | | CGAAATGACAGTTTACCGCCCTCTCTA |
| | | | | | | | | | ATGAATAACTGGCATGTTGTACTAAAAA |
| | | | | | | | | | TCGATGTTTTGCTTTGACAATCACCTGC |
| | | | | | | | | | TGTTTTGCGAAAACATTCGAGGAAGAA |
| | | | | | | | | | AAAACTGTGTTATGTATGTGCTGCATAA |
| | | | | | | | | | TCATGCATGTAAATACCATGTTTACC |
| 5.19 | 7.82 | 4.90 | 14.40 | 1E+06 | STM0962 | | | | |
| 4.40 | 9.12 | 3.63 | 14.04 | 1E+06 | IR STM0962- | ycaJ | paral | + | GCCCCACAAAACGCTACCGCTAGTGTA |
| | | | | | STM0963 | | putative | | AACGTTGCGGTAAGGTTATCTCTAAATA |
| | | | | | | | polynucleotide | | TGATGCTCCAGGTATCATGGCGTTGAT |
| | | | | | | | enzyme | | GATGAATCTCGTTATGCCTGATAGCAC |
| | | | | | | | | | GTTGCTTATGAGGTCCGCGGGTATAGC |
| | | | | | | | | | GCAATGGATGCGTTGTTGCTGTCGTCG |
| | | | | | | | | | GTCTGGTAAGGCGAAAACGTCGCTATT |
| | | | | | | | | | ACGTAAACGCGGTTTACGTTCATCAATA |
| | | | | | | | | | CAATCAGAGGCGATCATCAATTGATCG |
| | | | | | | | | | CGTTTCCTTTTATTATTCGATAAGCACA |
| | | | | | | | | | GGATAAGCATGCTCGATCCCAATCTGCT |
| 19.39 | 4.17 | 2.54 | 0.28 | 1E+06 | STM0974 | | | | |
| 4.76 | 3.09 | 4.28 | 4.25 | 1E+06 | IR STM0974 | focA | putative | − | CCTGGCTTATAGGCCCGTAAGTCGCAT |
| | | | | | STM0975 | | FNT family, | | GGCTTTTATGCAATTACGGTGTAACTTT |
| | | | | | | | formate | | TTGATTATCCTAATAAAAATAAATTTTAA |
| | | | | | | | transporter | | AAATTATAAATAGAGTTGAATTTTTTCCT |
| | | | | | | | (formate | | GACTCCTCCTGCTGCACGGTTAATTAA |
| | | | | | | | channel 1) | | TATGGAGTAATCAACAAATAAAGTAACA |
| | | | | | | | | | TCACTATGTCAATTAATTTAATATCAACA |
| | | | | | | | | | ACCAATATTTAACCTTGTTATTACATTTT |
| | | | | | | | | | TCGCCGTTTAGCGAAAATAAATAAAAC |
| | | | | | | | | | GGGGCCGCAAAGGCGCCCCGTAATAT |
| | | | | | | | | | AACGCAGCCGAGAGGGTAAACC |
| 6.85 | 5.88 | 0.71 | 8.94 | 1E+06 | STM1000 | | | | |
| 9.45 | 5.61 | 0.38 | 11.22 | 1E+06 | IR STM1000- | asnS | asparagine | − | CACCCATCCGCGCACGGTGACTTCTTG |
| | | | | | STM1001 | | tRNA | | GTCAACGGCTACGCGGCCCTGGAGTA |
| | | | | | | | synthetase | | CGTCGGCTACAGGCACAACGCTCATAA |
| | | | | | | | | | TATTCTCTCTAGTTAATAGTCGGAAAAA |
| | | | | | | | | | ATAAACACTTGTCCACCCGAAATGGGG |
| | | | | | | | | | GTATTCCTATGTTACCTGGCATCTGCAA |
| | | | | | | | | | TCAGACAAGCAGAAATCGCATCTGGAA |
| | | | | | | | | | GCAGGTTTTCAGAAAGAAACCTGTAAA |
| | | | | | | | | | AAGTTCGCACCTGCTCGCGAACCATTG |
| | | | | | | | | | AGAATTTAGGCTGGTTTTGCAAGCTTTG |
| | | | | | | | | | CGCACGTTACTCGATCAGGACGCGCAT |
| | | | | | | | | | CT |
| 6.14 | 5.36 | 0.30 | 7.51 | 1E+06 | STM1001 | | | | |
| 3.99 | 4.52 | 0.27 | 9.86 | 1E+06 | IR STM1019- | STM1019 | Gifsy-2 | + | TTTGATGCTGCTGCCGACAATTTTTAAC |
| | | | | | STM1020 | | prophage | | CGCGTCCGTGTGTCGCTCAGGGGGGT |
| | | | | | | | | | TACGTGGCAGAGGGAGTCCTATCAGAT |
| | | | | | | | | | CTTGCTGATAATTTGCGGGTGACTATAA |
| | | | | | | | | | CTGATGCTAAGGGAATAGAACTTTTGT |
| | | | | | | | | | CTTTTAGACTTGCATCAGGTGATCGCTA |
| | | | | | | | | | TATCCTATCAACCCAAAACGGTTCTGTA |
| | | | | | | | | | ACAAACCGAAAGCTATCAAGAGATGAT |
| | | | | | | | | | TTGTACTGGTCTAAGGATACCATTATGG |
| | | | | | | | | | AAGTTGTCAGAGAGATGGGCTCTAATA |
| | | | | | | | | | ATTGACTTAACAATAAGCACGCAATCA |
| 7.78 | 2.62 | 2.75 | 11.74 | 1E+06 | STM1070 | | | | |
| 13.38 | 4.07 | 4.15 | 9.95 | 1E+06 | IR STM1070- | ompA | putative | − | GTCTTTTTCATTTTTTGCGCCTCGTTAT |
| | | | | | STM1071 | | hydrogenase, | | CATCCAAAATACGCCATGAATATCTCCA |
| | | | | | | | membrane | | ACGAGATAACACGGTTAAATCCTTCAC |
| | | | | | | | component | | CGGGGGATCTGCTCAATAGTTACTCTA |
| | | | | | | | | | CCGATATCTACGGCTTATGCTGAGCAC |
| | | | | | | | | | CCCTGGCGATGTAAAGTCTACAACGTA |
| | | | | | | | | | GTTGGAAACTTACAAGTGTGAACTCCG |
| | | | | | | | | | TCAGACATGTGAAAAAAACATGACGGA |
| | | | | | | | | | TATACACATCATTTAACAGTTTCAGATG |
| | | | | | | | | | ATAAATCGTACAGCAAAAATTGCGGAA |
| | | | | | | | | | ACCGCTTCTGACAAGCGTTCTCGCAAAA |
| 8.17 | 1.31 | 2.77 | 2.51 | 1E+06 | STM1094 | | | | |
| 8.43 | 2.49 | 3.03 | 11.31 | 1E+06 | IR STM1094- | pipD | Pathogenicity | − | TAATGAAGGAGCCGTCAGCCGAAGCCT |
| | | | | | STM1095 | | island | | GATTGCCTACCAAAAGGGTAGTACAGG |
| | | | | | | | encoded | | CGATGACTTTACCCATACCCAGCAGCG |
| | | | | | | | protein: | | TAACGGCGAATGCAAGATACTTTTTCAT |
| | | | | | | | SPI3 | | AAAGGTTCCCACTGAATAACGCATTAT |
| | | | | | | | | | GGGATGAATTGACCCTGGATTGGAAAC |
| | | | | | | | | | CGAGAAAGTGATCGAGCCAGCAATATT |
| | | | | | | | | | CTTTGCCGGCATCCTTTATTTTCTCTTT |
| | | | | | | | | | ATTGAGGTTGTATTGATAACCACAGCC |
| | | | | | | | | | CTGTGGCAGGGAAGGGGAACAGAACC |
| | | | | | | | | | TGTCCTGACCTTAGCTATCACCACTATC |
| | | | | | | | | | AG |
| 7.07 | 2.68 | 3.49 | 14.57 | 1E+06 | STM1095 | | | | |
| 5.43 | 3.21 | 0.49 | 6.35 | 1E+06 | IR STM1119- | wraB | trp- | − | TGTAGCGATTCGCTACGTCTATTTAAAG |
| | | | | | STM1120 | | repressor | | ATATGCTCTCCTGTGAAGAGTGCAAATT |
| | | | | | | | binding | | TCAGCGCCATTTCTTTGATTTATAACAA |
| | | | | | | | protein | | TAATTAATTTGGCGACCTTTGTTGCAAA |
| | | | | | | | | | ATGATACATTTTTAAGCGCTTTGATTTT |
| | | | | | | | | | CCCAAATATAAGAATAACTTATTTATTTC |
| | | | | | | | | | TTATGGTTATTATTCTGCGTATTCGGCT |
| | | | | | | | | | TCCAATGTTGCAGAATATTTCGGTAAGC |
| | | | | | | | | | GGCCTACTACGACGTTTTTCACTATGCT |
| | | | | | | | | | TAATGTTACGCGGCGTTACTGATGATAT |
| | | | | | | | | | CGTTCATACGCTGCGCGAGG |
| 2.81 | 5.09 | 0.80 | 5.56 | 1E+06 | STM1120 | | | | |
| 5.74 | 4.54 | 2.14 | 8.31 | 1E+06 | STM1186 | | | | |
| 5.68 | 3.84 | 2.94 | 13.36 | 1E+06 | IR STM1186- | STM1186 | pseudogene; | + | CGGAAACCGCATCATTATTCCACTGCT |
| | | | | | STM1187 | | in-frame | | AACCTTGTTATAGCAAGATGACTTTTAC |
| | | | | | | | stop | | CATTTATCACCCGCTTACTCACAGTTTT |
| | | | | | | | following | | TTCACCAGCGTGAGCCAATCGCTTTAA |
| | | | | | | | codon 97; | | TAACCAGCAAAACCGCAGTGAAAAATG |
| | | | | | | | no start | | TTCATCCACTGGCGTAGACGTCTCTAT |
| | | | | | | | near coli | | AAGCATAGAAAAATGTGTGGCGCGAAT |
| | | | | | | | start | | CTCACAGGCTATTTAGAATCGCCCCCC |
| | | | | | | | | | ATGAAAACAGAAACGCCATCCGTAAAA |
| | | | | | | | | | ATTGTTGCTATCGCCGCTGACGAAGCG |
| | | | | | | | | | GGGCAACGCATTGATAACTTTTTGCGC |
| | | | | | | | | | AC |
| 5.68 | 2.96 | 2.94 | 12.77 | 1E+06 | STM1187 | | | | |
| 22.75 | 1.36 | 4.14 | 4.13 | 1E+06 | IR STM1224- | sifA | lysosomal | − | ATCGACCCTTTTTATCTCAACTGCGGG |
| | | | | | STM1225 | | glycoprotein | | CGCATCGGATGTAATATAATTTTTAAAA |
| | | | | | | | (lgp)- | | GAGACTGGCAATCAGTATAAAACCTGA |
| | | | | | | | containing | | GAGCTTCGCGTATAAACGCATTACTGT |
| | | | | | | | structures; | | CTGTGATAGCGTCGCTACAGGTAAAAA |
| | | | | | | | replication | | TAAAAGAAGGACTACCGCGGATGATGT |
| | | | | | | | in | | TGTAGATTTGCAATACTGGCGGCAACT |
| | | | | | | | macrophages | | TCTTTCATGCGTTTTTTATGCCGAAGGC |
| | | | | | | | | | ATGAAGTTTACCCTTGAATAAACTTCAT |
| | | | | | | | | | GCCTGGATGCGTGTGGATTTGTTAGCG |
| | | | | | | | | | TTGCGCAATTAATCGCTTATATCACTCA |
| 18.59 | 1.38 | 3.56 | 2.15 | 1E+06 | STM1225 | | | | |
| 11.41 | 3.53 | 2.69 | 5.70 | 1E+06 | STM1262 | | | | |
| 12.43 | 1.43 | 2.63 | 3.49 | 1E+06 | IR STM1262- | STM1262 | hypothetical | + | GGCCGCGTAATTTTTCTTCCGCCATTA |
| | | | | | STM1263 | | tRNA | | GCTCAACCGGATAGAGCATAGAGCTTC |
| | | | | | | | | | TACCTCTAAGGTTCGGGGTTCAATTCC |
| | | | | | | | | | TCGATGGCGGACCAGTTGATATCAAAA |
| | | | | | | | | | AAGGCCACCTGCGCGGTGGCCGCTGA |
| | | | | | | | | | GTTTCTGTTGAAATAAATGCAATGTTAT |
| | | | | | | | | | AATATAACAATCATCTTTCTAAGAAAGA |
| | | | | | | | | | TGAGGGTAACGTTTTGGTGATTCATTTA |
| | | | | | | | | | AAAAAACTGACAATGCTTCTGGGAATG |
| | | | | | | | | | CTGTTGGTAAATAGTCCTGCCTTCGCG |
| | | | | | | | | | CATGGTCATCATGCTCATGGCGCGCCG |
| | | | | | | | | | AT |
| 11.54 | 1.35 | 2.48 | 3.35 | 1E+06 | STM1263 | | | | |
| 13.02 | 1.20 | 2.58 | 5.66 | 1E+06 | STM1270 | yeaS | paral | + | |
| | | | | | | | putative | | |
| | | | | | | | transport | | |
| | | | | | | | protein | | |
| 15.43 | 1.23 | 2.41 | 5.51 | 1E+06 | IR STM1270- | | | | TTCTGGCGCTTTTGTAACCCACTATATT |
| | | | | | STM1271 | | | | GGTACCAAAAAGAAACTGGCAAAAGTG |
| | | | | | | | | | GGCAATTCTTTGATTGGCCTTCTTTTCG |
| | | | | | | | | | TCGGATTTGCCGCCCGGCTGGCAACG |
| | | | | | | | | | CTCCAGTCTTAACCACCTGGACCCGTC |
| | | | | | | | | | GTCAACGGCGGGTCATTGCTCTCCTTT |
| | | | | | | | | | CGGTTTTATTGCGTGGAAAACAGCAAA |
| | | | | | | | | | ATAGTAACCAATAAATGGTATTTAAAAT |
| | | | | | | | | | ACTGTTTTTGGAGCGTAACCTTTTTACG |
| | | | | | | | | | ACAGCGATGAGATTATCGCTGAGTAAC |
| | | | | | | | | | CTGCGTGAAGAGGGAAGCAAATGCGG |
| | | | | | | | | | CA |
| 13.99 | 2.43 | 2.21 | 7.19 | 1E+06 | STM1271 | | | | |
| 5.67 | 2.83 | 1.08 | 7.64 | 1E+06 | IR STM1311- | osmE | transcriptional | + | CGCTGGATGATACCGGGCACGTGATTA |
| | | | | | STM1312 | | activator of | | ACTCCGGCTACCAGACCTGTGCGGAGT |
| | | | | | | | ntrL gene | | ACGACACTGACCCACAGGCGCCGAAG |
| | | | | | | | | | CAGTAACAACTGTACATTGCCTGAACAT |
| | | | | | | | | | TCAAGGAAACCGGCCTGCGAGCCGGT |
| | | | | | | | | | TTTTTTGTGCCTGCCATAACCTTATTTA |
| | | | | | | | | | TTATCGCGAATTATTTGCCCGAAATGTG |
| | | | | | | | | | AGGGGGGTCATAACGCCAGGTCAATG |
| | | | | | | | | | AGAGACAATTTAGTGGGTCAAGGAAAT |
| | | | | | | | | | ACCATCCGGTGGTCCGATCCCGTATAC |
| | | | | | | | | | TCATTTCAGCCACCTAAAAAAGTAAATC |
| | | | | | | | | | CGG |
| 3.10 | 2.03 | 2.19 | 3.50 | 1E+06 | IR STM1360- | ydiN | putative | − | TTATTGCATTGATAGCATTTCATTTGTTA |
| | | | | | STM1361 | | MFS family | | GCCAGGAAATATAAAAATTGCTGCGAA |
| | | | | | | | transport | | TTTGTTGTTTAATACATATAACTCGTGA |
| | | | | | | | protein | | TGCTCATCGCAATTTTTCTGATAAGTGT |
| | | | | | | | | | GAAGATAATGAATAATAATTAACACGAA |
| | | | | | | | | | AATTACATTTTTTGTTTCCCGGTGATAA |
| | | | | | | | | | TGGCTAACGTTTTATTTTGCATAGCAAG |
| | | | | | | | | | GCAATAATATTGCAACTGGCACGCTAA |
| | | | | | | | | | CATTTATTGCGCGGTTGACGCTGCTTC |
| | | | | | | | | | AGCGTGATGTTGTGATTCAGCCCGACT |
| | | | | | | | | | TCGGTAACCGATGAACAGTGCGAG |
| 4.06 | 6.04 | 2.68 | 4.86 | 1E+06 | STM1361 | | | | |
| 5.49 | 3.54 | 0.64 | 6.24 | 1E+06 | STM1364 | ydiK | putative | − | |
| | | | | | | | permease | | |
| 5.96 | 2.50 | 1.73 | 12.49 | 1E+06 | IR STM1364- | | | | GCTGTACTATCCACAAACAGGCCACAA |
| | | | | | STM1365 | | | | TCATGATGGCTAAAAACAGCACCGATA |
| | | | | | | | | | GCAGCACTTGCGCAATATCCCTGGGCT |
| | | | | | | | | | GACGAACATTTACCATAAATACTTTTCA |
| | | | | | | | | | CCTTTGTCTTTGCGCCAGAACGTTGGC |
| | | | | | | | | | GCGACGTGAACATGCAAACCACACCCT |
| | | | | | | | | | ATAATGATGAGCAATTTCAGCGGTTTTT |
| | | | | | | | | | AACAGGCCGATTCTGCATGTAATTCTG |
| | | | | | | | | | TTGGGCGCACAGGAAAAAAATGTGATA |
| | | | | | | | | | CAACAAATAACGCAACACGCAAACGAT |
| | | | | | | | | | TAAGCATCCCTTCCTGTGCGTAGACCG |
| | | | | | | | | | CT |
| 11.27 | 3.11 | 0.89 | 6.43 | 1E+06 | IR | lpp | murein | − | TGATCGATTTTAGCGTTGCTGGAGCAA |
| | | | | | STM1377- | | lipoprotein, | | CCAGCCAGCAGAGTAGAACCCAGGATT |
| | | | | | STM1378 | | links outer | | ACCGCGCCCAGTACCAGTTTAGTACGA |
| | | | | | | | and inner | | TTCATTATTAATACCCTCTAGATTGAGT |
| | | | | | | | membranes | | TAATCTCCATGTAGCGTTACAAGTATTA |
| | | | | | | | | | CACAAACTTTTTTATGTTGAGAATATTTT |
| | | | | | | | | | TTTGATGGGAATGCACTTATTTTTGATC |
| | | | | | | | | | GTTCGCTCAAAGAAGCATCGAAATGCA |
| | | | | | | | | | TGAAAGTCCCTAAAAAACCGAAAGAAA |
| | | | | | | | | | ACAGGGGGCTTCCATCGGATTCTTCTT |
| | | | | | | | | | AGATAATCCGCAATTAGATAGTAAAA |
| 12.11 | 2.11 | 5.46 | 4.68 | 1E+06 | STM1389 | | | | |
| 14.05 | 3.53 | 5.48 | 6.58 | 1E+06 | IR | orf319 | putative | − | CTTATGTCCGCCATCAAAGCGTACCGT |
| | | | | | STM1389- | | inner | | GGCGCCAGTCAGACATCCGCTAATGCC |
| | | | | | STM1390 | | membrane | | GACTACGGGTTTGTTATTCATGATTCCC |
| | | | | | | | protein | | CCTTATTGAAAGTACGACGACTGACGC |
| | | | | | | | | | CAATGGCGCAAAATGTTATCTCACGCT |
| | | | | | | | | | GATTTAAAACTTACACAACTTTGTTTTTT |
| | | | | | | | | | TGTCTAAGTTTTCGCGGAGATTTTTTTT |
| | | | | | | | | | GACGTAATTAAATATCAATAAGATAGAA |
| | | | | | | | | | TGAGGGGAAGAAATCTATTTCAGCGCC |
| | | | | | | | | | TATAGTGTGATAACCTCCAGCGAAGCG |
| | | | | | | | | | ACCACGTTGCGCCACTGGGCAAGCTG |
| 14.85 | 3.17 | 5.44 | 8.13 | 1E+06 | STM1390 | | | | |
| 8.78 | 2.81 | 2.05 | 9.37 | 2E+06 | STM1437 | | | | |
| 4.15 | 1.85 | 4.61 | 5.34 | 2E+06 | IR | ydhM | putative | − | AAAACGACCCTTTAGGCACTTGGGCGG |
| | | | | | STM1437- | | transcriptional | | TTTTGAGCAACTCGCTAAGCCCCATGC |
| | | | | | STM1438 | | repressor | | CGGTAAAACCCCGTTGCATACAAAGCT |
| | | | | | | | (TetR/AcrR | | GCTCGCCGGTGGCCAGCAGATGTTCG |
| | | | | | | | family) | | CGGGTATCGTGTTCGGTTTGCTTATTC |
| | | | | | | | | | ATAGCAGGCAGTATAGTAGACCAGTCG |
| | | | | | | | | | GTCTACTACAAGCAGAGTTGCCATAAT |
| | | | | | | | | | GTCAGTTAGCGTCTTCAATAGTCATAAG |
| | | | | | | | | | CGTCAAACGTTGAGGAGGGGATGTGG |
| | | | | | | | | | CCGAGCAGTTGGAGTTTTTTCCTGTAG |
| | | | | | | | | | CAAGCCCATGTCGCGGTATCTGCCAGT |
| | | | | | | | | | CTGAT |
| 7.00 | 3.17 | 3.39 | 4.75 | 2E+06 | STM1463 | | | | |
| 9.41 | 3.20 | 4.26 | 6.11 | 2E+06 | IR | add | adenosine | − | TCAAGGTGGCGGTGGATGTCAGTCAAA |
| | | | | | STM1463- | | deaminase | | GGAAGCGTAATATCAATCATGGGCGCA |
| | | | | | STM1464 | | | | CTCAATTTTTAATAAAAGTGCGCACCAT |
| | | | | | | | | | TATACTACAGATTGATAATGCTCTGGAA |
| | | | | | | | | | ATTTTGCAAAAACGGAGTCATTACGTTG |
| | | | | | | | | | CAACTTCGCGAGAGCGCGGGAGAAATT |
| | | | | | | | | | TTGTATCATTCTCTTTAACGCGCCCCCG |
| | | | | | | | | | GTCAGCTCACGGGGGCGTCTCTGTTAT |
| | | | | | | | | | CGCCTCTCAGGATAAAGGGTCAACCCC |
| | | | | | | | | | CCGCCTGTAGACAGTATCAGCGAACGG |
| | | | | | | | | | TGCGGTGGCAAAATCCATATCCGAGAT |
| 8.15 | 2.46 | 3.30 | 6.09 | 2E+06 | STM1464 | | | | |
| 8.84 | 3.81 | 4.45 | 7.93 | 2E+06 | STM1475 | | | | |
| 12.95 | 2.78 | 5.34 | 7.26 | 2E+06 | IR | rstA | response | − | TCACCACGCGGCTCAACAATGACATCA |
| | | | | | STM1475- | | regulator in | | ATATCATGTTTCGCCAGATAAGCGGCA |
| | | | | | STM1476 | | two- | | ATGAGAGAACCCACTTCAGCGTCGTCT |
| | | | | | | | component | | TCAACAAATACAATGCGGTTCATATTAT |
| | | | | | | | regulatory | | AAATGGAGAATAGAAAACGCCAACATA |
| | | | | | | | system | | CACCGCCTCTGTTTTCCCTTCCATAAAT |
| | | | | | | | with RstB | | CTTTTCTAAACGAGAGCGGTTCCGTTAT |
| | | | | | | | (OmpR | | GCTACACGCTGTTGTTATTAGCGTGTTA |
| | | | | | | | family) | | AGGCAAGGTAATGGGACTCGTGATTAA |
| | | | | | | | | | AGCTGCCCTGGGGGCGCTGGTCGTCG |
| | | | | | | | | | TATTGATTGGTCTGCTGTCAAAAACGAA |
| 12.88 | 2.12 | 5.34 | 5.77 | 2E+06 | STM1476 | | | | |
| 13.06 | 6.41 | 3.01 | 5.77 | 2E+06 | IR | yncB | putative | − | CTTGCGTGATATTCTCATCTTTTACAAC |
| | | | | | STM1588- | | NADP- | | AATACAGGTTTCTTTATGGCAACCGTTT |
| | | | | | STM1589 | | dependent | | TATCTCCGTCATTCCTTCATGTATCGAG |
| | | | | | | | oxidoreductase | | ATTTTTGACCGGTTCAGGCCGCTGAGG |
| | | | | | | | | | GAGATAAGCTGCCCCACCGCGATCTGA |
| | | | | | | | | | ATGATGAATATAAGTAAAGCCGCAATTT |
| | | | | | | | | | TAAAATTTGCACATTTTTATGGCGACAT |
| | | | | | | | | | AATGCCGCCATTTTTTCTTTACGCATCG |
| | | | | | | | | | TCCGCTAAACGTATCACGACTTTGCCA |
| | | | | | | | | | AAGTTCTTCCCCGCCAGCAGCCCCATA |
| | | | | | | | | | AACGCTTCTGGCGCATTTTCCAGCC |
| 12.88 | 6.41 | 2.39 | 6.58 | 2E+06 | STM1589 | | | | |
| 6.40 | 4.19 | 4.85 | 7.12 | 2E+06 | IR | nifJ | putative | + | ACGCAATGGCCCAGCGACAAAATGAAT |
| | | | | | STM1651- | | pyruvate- | | ATGTGACAATAAAGGCATATAACAGGC |
| | | | | | STM1652 | | flavodoxin | | GTAGAATATCGTAACCGAATGATATTGT |
| | | | | | | | oxidoreductase | | ATAATTTTTATTTTGTATAATACCCCCAA |
| | | | | | | | | | AAGCATTCGTATAAATTATATCTATTTCA |
| | | | | | | | | | CTGCGAATTATTTCATTAATTATTGAATT |
| | | | | | | | | | AAACGGTAACATCTCTTTTTAGGTCTTT |
| | | | | | | | | | CCTGACAAGGCAGAAATAACGTTTTAA |
| | | | | | | | | | CGTCAACTCGCTGATTATTTACGTGGA |
| | | | | | | | | | ATACGCGTAATATTACGTCGCCCTCCC |
| | | | | | | | | | CTGTAGGTAGTCCCCGCAGAGTA |
| 4.08 | 3.17 | 4.01 | 5.20 | 2E+06 | STM1652 | | | | |
| 2.87 | 2.35 | 8.22 | 8.30 | 2E+06 | IR | ychE | putative | − | ATGTTCGTTAATGATCAAAACGCGCAG |
| | | | | | STM1748- | | integral | | AAGATACGCCTTTTATTCGCATAGTTCA |
| | | | | | STM1749 | | membrane | | CCTCTTATCTACGCCTAATTTCATCCAT |
| | | | | | | | proteins of | | TCATCGCTGTTATTTATATGTACTCGTT |
| | | | | | | | the MarC | | ATGCTAATCCACTCACTCTTCATGATAA |
| | | | | | | | family | | CGATTTCTTAACAATTTACATAAAAGGC |
| | | | | | | | | | TAAAATGGCCTGCTGAAAGGTGTCAGC |
| | | | | | | | | | TTTGCGTAATCTTGATTTAGATCACACA |
| | | | | | | | | | ATCGCTACTCAGAAGTGAGTAATCTTG |
| | | | | | | | | | CTTACGCCACCTGGACGTAACGCGTTA |
| | | | | | | | | | GAGTTAAATGATACTAACGCAGAAG |
| 3.34 | 1.80 | 4.30 | 3.36 | 2E+06 | IR | galU | glucose-1- | − | CCCAATCCCGCGACCGGGATAACGGC |
| | | | | | STM1752- | | phosphate | | TTTTTTGACTTTCGAATTAAGGGCAGCC |
| | | | | | STM1753 | | uridylyltransferase | | ATTTAAAATTCTCCTGGACTGTTCATGT |
| | | | | | | | | | ATTGAACGTGTTCATTAATCTGTATCGT |
| | | | | | | | | | GTTCCAGTATATCAGTACCAGAACAAG |
| | | | | | | | | | CCTCAGGTCCAAAAAGGACTTATATTG |
| | | | | | | | | | GTATAATTAAGACAAATACTTATAAATC |
| | | | | | | | | | TGCCGCAGATAGTAACACTCGTCGGGA |
| | | | | | | | | | AAGGCCGGTAAAGCAATTTCCGCTCAC |
| | | | | | | | | | TCTTCCGTTTGGTCATTCCGCAGACAA |
| | | | | | | | | | CATCAATCGCAGACGCCCTCCTGCGCCC |
| 3.37 | 3.21 | 4.25 | 6.30 | 2E+06 | STM1753 | | | | |
| 19.52 | 7.93 | 7.59 | 11.87 | 2E+06 | STM1785 | | | | |
| 20.40 | 9.07 | 9.65 | 17.70 | 2E+06 | IR | STM1785 | putative | − | ACGTCCCGAAAAAAATGAATCAAATAAT |
| | | | | | STM1785- | | cytoplasmic | | CGGATAAGTCAAATCTGATGTTATTTTT |
| | | | | | STM1786 | | protein | | CATGGGACGCCCTCTTTCAAACAGTCT |
| | | | | | | | | | CTTTTTTGCATTCCTTTAAAACCAGCAT |
| | | | | | | | | | CACTATTTTATATAAAAATCATCACGAA |
| | | | | | | | | | GTATGCTTCTTTTAACGATGACCTCAAA |
| | | | | | | | | | TCCTCCCCCCTTTTGCATCAACTTACGC |
| | | | | | | | | | ATCCCTGAAATGGCGAGAACAGGCTAA |
| | | | | | | | | | ATCTACCCGAGGTCACTCGCTAAAAAC |
| | | | | | | | | | CTCATCCTGGAACAAGCTCAACCGCCC |
| | | | | | | | | | TTCCCCGCTACGGCCCTTTCGCCGA |
| 11.00 | 2.99 | 0.32 | 6.05 | 2E+06 | IR | STM1794 | putative | + | CCCGCCGACAGGACGACATAACATTGA |
| | | | | | STM1794- | | homologue | | TACATGTCGTTATCATAACGTTTACTTT |
| | | | | | STM1795 | | of glutamic | | TAGAGGTGCGTCATAATTATGACAAATA |
| | | | | | | | dehyrogenase | | GCCACCTTGCACATATTTCGCATATTTA |
| | | | | | | | | | AGCAATTAATTGCATAATTAGCAATATA |
| | | | | | | | | | TCACCTCTTATAGCGGATAGTTAACCAC |
| | | | | | | | | | TTCCCATCCAAAATCATAACGAAAATCC |
| | | | | | | | | | AACTGCCTGCCATTTTTGATCTGAGTTA |
| | | | | | | | | | ATTGTTTAAAAAAGTGTTAAATTTATCG |
| | | | | | | | | | CTACATGGTGTGATCTACTATGTACCAC |
| | | | | | | | | | GGTCAATTAAAGAACATATTAC |
| 10.76 | 3.19 | 0.36 | 5.54 | 2E+06 | STM1795 | | | | |
| 8.86 | 4.20 | 0.89 | 13.00 | 2E+06 | STM1813 | | | | |
| 8.17 | 4.02 | 0.89 | 14.31 | 2E+06 | IR | ycgL | putative | − | CGAATCCTTTCATCAACGCTTCAGGCA |
| | | | | | STM1813- | | cytoplasmic | | CCCGCGAAAAATCGTCTTTTTTTTCGAC |
| | | | | | STM1814 | | protein | | ATACAAATAGGTTTGATCGCGCTTGCTA |
| | | | | | | | | | CTTCTATAGATCACACAAAACATACTTT |
| | | | | | | | | | TACTCTGAATTAACGGGATGGTGACTT |
| | | | | | | | | | GCCTCAATATAATACTGACTATAACATG |
| | | | | | | | | | CCTTCTGGACTTCGGAATATCACTCCG |
| | | | | | | | | | TATCGGAGATGATAAATAGCAAATTGA |
| | | | | | | | | | GTAAGGCCAGGATGTCAAACACGCCAA |
| | | | | | | | | | TCGAGCTTAAAGGCAGTAGCTTCACCT |
| | | | | | | | | | TATCAGTGGTTCATTTGCATGAAGCGG |
| 7.85 | 3.58 | 0.82 | 13.13 | 2E+06 | STM1814 | | | | |
| 5.50 | 8.38 | 4.89 | 4.63 | 2E+06 | STM1839 | | | | |
| 5.50 | 9.75 | 4.99 | 5.51 | 2E+06 | IR | STM1839 | putative | − | CAATAACGCTTCGAGCAATTCTATCTGC |
| | | | | | STM1839- | | periplasmic | | TCGTTGGCACGGGAGCTTGCCCGGTT |
| | | | | | STM1840 | | or exported | | GACAAAGAACCAGAGCGCCAGCCCCA |
| | | | | | | | protein | | CCACCAGAACCACCATTGATACTATTAA |
| | | | | | | | | | AGATGCAAGAGAAAACGCACCAGAGTT |
| | | | | | | | | | TAAAACGTCGTTCATTTCACCACCTCAA |
| | | | | | | | | | TGTAGAGACGTCATTCTACCACTGCTA |
| | | | | | | | | | CACGGGAAGGAAATCTCTGGTGTAAAA |
| | | | | | | | | | CGTTTACCAGGGAATAAATTTATTGATG |
| | | | | | | | | | GCGCAAATACCGCTGAAAAATTGTACA |
| | | | | | | | | | TCCTGATCGCACATGATATTAAACACCTG |
| 5.70 | 7.66 | 4.99 | 8.75 | 2E+06 | STM1840 | | | | |
| 4.69 | 4.19 | 4.44 | 7.68 | 2E+06 | IR | yobG | putative | − | AATTGTACATCCTGATCGCACATGATAT |
| | | | | | STM1840- | | inner | | TAAACACCTGCGCCCACAGCAACAGGC |
| | | | | | STM1841 | | membrane | | ATACTACCACCACGATGCCGAGAACGA |
| | | | | | | | protein | | CCCATCGAAATTTTTTCACTCCACTCTC |
| | | | | | | | | | CGATCTTACATCTTATGTCGCTAAATTA |
| | | | | | | | | | TCATGAGTTACTTAAACCAGGAGTAACT |
| | | | | | | | | | GTAGCGGCATTATATGTTTTTAGGAATG |
| | | | | | | | | | ATTCACTTGTTTCAATCAATGTACACGC |
| | | | | | | | | | TACTCTTATTCTAACTAAAAAAGAAAAG |
| | | | | | | | | | AGGTAGTAATGCGTTTGATCATTCGCG |
| | | | | | | | | | CAATTGTATTGTTTGCCCTGGTGT |
| 3.83 | 2.95 | 3.54 | 4.78 | 2E+06 | STM1841 | | | | |
| 12.66 | 3.22 | 3.87 | 6.92 | 2E+06 | IR | sopE2 | TypeIII- | − | AAACTACAAATGAAATGGATTGACGCAT |
| | | | | | STM1855- | | secreted | | CTATTAGTGGTCAAAAAAACGCGCTAC |
| | | | | | STM1856 | | protein | | GAGAAATAATCAGTAACAATTGCAACAC |
| | | | | | | | effector: | | TATTCCAATCATAACGTAAACTATATGA |
| | | | | | | | invasion- | | TACCAGGTGATTATTATTGCTTTTAGGT |
| | | | | | | | associated | | AACATATCTGTATGGCTGCTTTTAAGCA |
| | | | | | | | protein | | ACAATACTCTAACACAACATATAACATT |
| | | | | | | | | | ATAACTTACAATAGGTTAACAAATGGAA |
| | | | | | | | | | TTACAGCTTATGCTTAACCACTTTTTCG |
| | | | | | | | | | AGCGCGTCAGAAAGGATGCAAATTTCA |
| | | | | | | | | | ACGCATTTCTAATCGATCTGGAA |
| 11.89 | 3.22 | 3.87 | 7.20 | 2E+06 | STM1856 | | | | |
| 19.06 | 3.74 | 0.57 | 7.84 | 2E+06 | IR | STM1866 | pseudogene | − | TGATTTAATAAGAGAAAACATATTATTA |
| | | | | | STM1866- | | | | CCCTCATAGTAAGCAGTATTAAATAAGC |
| | | | | | STM1867 | | | | CGGGATATATCTGATGTTCAATCAGTC |
| | | | | | | | | | CCTCATATAGGGTTAGCACCATAGCGA |
| | | | | | | | | | GTCGTTTTCACAAAAAACACAGACTGTT |
| | | | | | | | | | GAAACTTTATTTATCACTTTGACATTTG |
| | | | | | | | | | CAATACATGACACATGATTAGCTTCAGC |
| | | | | | | | | | CGCCATTATAGGGAAAGCTCCATTTCC |
| | | | | | | | | | ATACTCATTTACTCACTTCTCCCTGCGG |
| | | | | | | | | | AAAAAGAAATGCAGTATAGCCAGCGTG |
| | | | | | | | | | GTGCTTTTGCTGAAACCAGGCGCGA |
| 5.10 | 5.03 | 3.26 | 16.52 | 2E+06 | STM1933 | | | | |
| 4.54 | 5.03 | 3.36 | 16.19 | 2E+06 | IR | STM1933 | putative | − | ATGTACGTCAGGTGATGGTCATTTTCG |
| | | | | | STM1933- | | ribose 5- | | TCGCACATGCCGACGTTAAAAACGGGA |
| | | | | | STM1934 | | phosphate | | AATCCCTTTTCATTGGCGACGGCGCTA |
| | | | | | | | isomerase | | AGTTCGTTATAAATGATGGCATTTTTGC |
| | | | | | | | | | TGGCCTGGCTATTTTCCATCATCAGTG |
| | | | | | | | | | CAATTTTCATCGTGTTTCTCCTGAATGC |
| | | | | | | | | | AGACGGTCGCGCCTGCGTAAATCATGA |
| | | | | | | | | | CGTTTTACCCACATTACACATTTGAGAA |
| | | | | | | | | | CACACATTCAAATTTAATAAAACCAGGT |
| | | | | | | | | | TTCATTAAATGAAAAGACGCTCACACAT |
| | | | | | | | | | TTTCTGTTCCCGCTGTAAATCCCCTG |
| 3.30 | 3.86 | 0.86 | 10.98 | 2E+06 | STM1957 | | | | |
| 3.72 | 2.84 | 0.98 | 6.19 | 2E+06 | IR | tnpA_2 | transposase | − | TTAATATGCTGCCTACTGCCCTACGCTT |
| | | | | | STM1957- | | for IS200 | | CTCTCCATAGAACGCTTGTCTTCGGTAT |
| | | | | | STM1958 | | | | TTGGGCGCGAAAACTATGTGATATTTA |
| | | | | | | | | | CAGTTCCATCGGGTGTGCGCTAAGCTC |
| | | | | | | | | | TTTTCGTCCCCCATTGGGACCCCCTTTT |
| | | | | | | | | | GATTTCTTGTTGAACTTTTGCAGTTGCC |
| | | | | | | | | | AGACCGCAAGATGTTTTAACAAATCAAA |
| | | | | | | | | | AGGGGTTTTAATAACTGGCTTAAAGCT |
| | | | | | | | | | GAAAGCTTTCCGGAACCCCCAGCCTAG |
| | | | | | | | | | CTGGGGGTTTTCCATAGACAATAAACG |
| | | | | | | | | | GGATGCGCAAAAGCCCACCCCGAACA |
| 5.77 | 1.84 | 4.86 | 5.12 | 2E+06 | STM1966 | | | | |
| 6.40 | 3.52 | 5.94 | 5.51 | 2E+06 | IR | yedF | putative | + | ATTCCACTGGATGCGCGCAATCACGGC |
| | | | | | STM1966- | | transcriptional | | TATACGGTGCTGGATATCCAACAGGAT |
| | | | | | STM1967 | | regulator | | GGCCCGACAATTCGTTATCTGATTCAA |
| | | | | | | | | | AAATAAGCGCATACTCCCGCTGTACGT |
| | | | | | | | | | TACGGCGGGAGACCTTTTACGGCATAA |
| | | | | | | | | | CCGGCAAAAATCTACAACGCATAAAAG |
| | | | | | | | | | AAATCAGACAAGGTCGTCTTGTGCGCC |
| | | | | | | | | | GTGGCATAAATCTATTATATAACGTATA |
| | | | | | | | | | CCGTTTTAATTCTGTCTGAGCCGATGAA |
| | | | | | | | | | AAATCCAGGGTTATTTTAATCAAAACAT |
| | | | | | | | | | AAAACAATTATTATTTTCCGTCTACGCC |
| 5.61 | 3.99 | 3.98 | 9.77 | 2E+06 | IR | thiM | hydoxyethylthiazole | − | TCAGACTTCCCTACGCTGGCATTATCC |
| | | | | | STM2147- | | kinase | | AGATCAGGTGGTACGGGTATTTCTCAG |
| | | | | | STM2148 | | (THZ | | CCTTCACAAAGAAGGGCACCCCGAGTC |
| | | | | | | | kinase) | | GTCAAGCCCCACCGTGTTAAGCGGGG |
| | | | | | | | | | TTTCGCTATTAAGCATACTGTCTGTGCC |
| | | | | | | | | | AGACAATGTAAATTTACAGTCAGCGGC |
| | | | | | | | | | GGACGATAATTTCAGCGTTATCAGATA |
| | | | | | | | | | GTTCTCAAAACCTATTCGGTTCTGGCAA |
| | | | | | | | | | ACTTGCTGGCGGATATGTTGCTGCACG |
| | | | | | | | | | ACGCTTTCGTTTACACTTTTTACGAAAA |
| | | | | | | | | | GGGGCGTGAGATAACAAAATAGCGCTT |
| | | | | | | | | | GT |
| 8.35 | 4.88 | 0.85 | 5.87 | 2E+06 | IR | yehU | paral | − | AACTCGTACATACCCGCAAACCACACT |
| | | | | | STM2159- | | putative | | TCAATTAAAAGCGCGTAACATACATTGA |
| | | | | | STM2160 | | sensor/kinase | | GTACGATTAACTTTCTTTGAACTGTTGC |
| | | | | | | | in | | ATAAAAATATGAATTCGTGAATACGATC |
| | | | | | | | regulatory | | ACTTAAACGCCGCGCCGCAACCCGCTA |
| | | | | | | | system | | CTTCGCGTTTTAATGCATAAAAAACAGG |
| | | | | | | | | | CAAAACTTCCTGGTTCCTAAAAGAGCG |
| | | | | | | | | | TCTAAAGTTAAACCGGGACCTCGCGAG |
| | | | | | | | | | CAAGGGTGAAACGATGGCGCTTTACAC |
| | | | | | | | | | AATTGGTGAAGTGGCTTTGCTTTGTGAT |
| | | | | | | | | | ATCAATCCTGTCACGTTGCGCGCGTG |
| 9.38 | 3.01 | 0.67 | 7.05 | 2E+06 | STM2160 | | | | |
| 14.27 | 3.59 | 10.29 | 16.23 | 2E+06 | STM2180 | | | | |
| 11.49 | 3.86 | 11.30 | 17.89 | 2E+06 | IR | STM2180 | putative | + | CGCAACGCTATGCCAGCCAGGGGCAA |
| | | | | | STM2180- | | transcriptional | | CTGGCGATTTTAAACTTGCCAAAAATTG |
| | | | | | STM2181 | | regulator, | | AGCAAAAAGGCAGCGTAGGGATGTTCT |
| | | | | | | | LysR family | | GGCGTAAGAATGAGACGCCGTCTTTGG |
| | | | | | | | | | CCCTGAGTCGCTTTTTGTATTTTTTAGC |
| | | | | | | | | | CCAGGTTTAGCGCCGCCGACCAGGGG |
| | | | | | | | | | CATTGCCCGATGTTCCTGCTGTCTATA |
| | | | | | | | | | CCCACTATGCTAAGAATTCATGATGTGA |
| | | | | | | | | | TCGGTAGCACGTTTTAACGTTTAATTGT |
| | | | | | | | | | ATGATGAATCCATCTCATCAAGGGCTTT |
| | | | | | | | | | AAACATGAGTAAGTCACTGAATATTATC |
| 3.94 | 3.73 | 0.47 | 5.79 | 2E+06 | STM2226 | | | | |
| 5.04 | 2.26 | 0.41 | 4.33 | 2E+06 | IR | yejK | nucleotide | − | GCGCTTGATAAGCTGGTGCAGGGCAAT |
| | | | | | STM2226- | | associated | | CTGGTTGATATCCAGACTCATGATAAAC |
| | | | | | STM2227 | | protein, | | TCTCCTTTAAGACCGGGCGGTATTCAA |
| | | | | | | | present in | | CCACCGCCTGCCGGAAGACGCAAGCA |
| | | | | | | | spermidine | | ATCGCCCTGTCATTTCAGGCGTTATCC |
| | | | | | | | nucleoids | | GTAACGCGAATGATTTAGGGGATAAAA |
| | | | | | | | | | ATGCAGAAAAAAAACTGTTGCTACGGT |
| | | | | | | | | | AATATGTTGCCCTTTCATGAACAAACAG |
| | | | | | | | | | ATTTTGATTTATGCCACAACTCTCCCGC |
| | | | | | | | | | TATAGTGATGAACATGTTGAACAACTGC |
| | | | | | | | | | TGAGCGAACTGCTCAGTGTACTGGAAAA |
| 4.73 | 2.38 | 0.36 | 3.82 | 2E+06 | STM2227 | | | | |
| 6.87 | 2.44 | 5.79 | 5.78 | 2E+06 | STM2280 | | | | |
| 13.11 | 3.72 | 5.26 | 12.44 | 2E+06 | IR | STM2280 | putative | − | CAAAAAAGATAATAAAACTGACTATGGT |
| | | | | | STM2280- | | permease | | GATTGCCCAAAAATCTTTCGTCCATAAT |
| | | | | | STM2281 | | | | TTTTCTTTCATTCTTAACGACCCGCTCA |
| | | | | | | | | | GATGGCGCACGCAGGCAACGCTCAGC |
| | | | | | | | | | TCAACTGAACACCTATCAGGTGCGTCA |
| | | | | | | | | | AAATGTGATGTATTCGATAGAATCACAG |
| | | | | | | | | | TATAAACAAGTGCACTCTATTAGAAAAA |
| | | | | | | | | | TTAATCGTTTTAATTATATTGATTAGGTT |
| | | | | | | | | | TTACTAATGACACTAACCCAAATCCACG |
| | | | | | | | | | CCCTGCTTGCCGTACTGGAGTACGGC |
| | | | | | | | | | GGATTTACCGAGGCCAGCAAACGGC |
| 11.78 | 4.41 | 5.49 | 12.44 | 2E+06 | STM2281 | | | | |
| 16.05 | 5.97 | 5.10 | 11.78 | 2E+06 | IR | lrhA | NADH | − | AATACCAAATGCAACTGATCGGGATAT |
| | | | | | STM2330- | | dehydrogenase | | ATCAAAGAGAATTTGTCATACCTTTAGG |
| | | | | | STM2331 | | transcriptional | | CGTCTACAGATTTCTGCTAATGATGGA |
| | | | | | | | repressor | | CGTGTAAATCTTGTAACAGCGTCAAATA |
| | | | | | | | (LysR | | GTTTACCGAGACGCACAGATACAAAAA |
| | | | | | | | family) | | CAATATATTGAACAATAGGTTATGTATA |
| | | | | | | | | | AAATCGCGTCATGATAATTAGCAGACA |
| | | | | | | | | | ACGCAGACTACGCCCCCGTTTCGGATC |
| | | | | | | | | | ATTATCTTAACCTAAAACCGCTATATTT |
| | | | | | | | | | ATAAGTATTATTACGAATAATCTTAACC |
| | | | | | | | | | TGGGATATGTTATACTAATCGGACCA |
| 3.75 | 2.85 | 0.51 | 3.73 | 2E+06 | STM2387 | | | | |
| 5.29 | 2.67 | 0.65 | 3.05 | 2E+06 | IR | sixA | phosphohistidine | − | ACCCACAAGGGGTCAAGGGACGAACC |
| | | | | | STM2387- | | phosphatase | | GAATCACTGGCGGCATCGAGGGCTGC |
| | | | | | STM2388 | | | | GTCGCCGTGACGCATGATAAAAACTTG |
| | | | | | | | | | CATATTGCACCGCTTTTGTTAACCAGTT |
| | | | | | | | | | TCACCAACACGCTTACCACATGCCCCT |
| | | | | | | | | | ATTGGCTGCGGCAAAAATGCGGTGGC |
| | | | | | | | | | CGGCATTGTGCCTTATCCATTCACTGA |
| | | | | | | | | | ATGAAACGCTGTTTTTTACCTCAATGGC |
| | | | | | | | | | GTAAGTATAGTCAATCCTTGATTATTAT |
| | | | | | | | | | TTCGCCACTAAGGAGGCATTCAGTGCG |
| | | | | | | | | | GATTCATATTCTCTTTGACCTCAATTTC |
| | | | | | | | | | CCT |
| 5.41 | 1.95 | 3.44 | 6.00 | 3E+06 | STM2408 | | | | |
| 8.14 | 3.92 | 5.34 | 6.93 | 3E+06 | IR | mntH | Nramp | − | GGGTACGGGTGATTACTTTGATAGTGT |
| | | | | | STM2408- | | family, | | GAAACGATAGACCGATACGATGACGAC |
| | | | | | STM2409 | | manganese/ | | CTGTATCAGAACAGTTTGGCTTAACATT |
| | | | | | | | divalent | | ACAAGATTAGCACACTGATATAACTTTT |
| | | | | | | | cation | | CATTTTCATATTCAGTACAGTAAAAGTG |
| | | | | | | | transport | | TATTACAGATCACTAATTTTGAATCTCG |
| | | | | | | | prortein | | TCACAGGTCCTTATTATAGTGTGTGTTG |
| | | | | | | | | | GATCTCGTTTTCTTTACGGCTGTTGCAT |
| | | | | | | | | | AGAATGTGCACGAAAATTAAACCTGCC |
| | | | | | | | | | TCATATTTGGAGCAAATATGGACCGCG |
| | | | | | | | | | TCCTTCATTTTGTCCTGGCGCTTGC |
| 8.86 | 3.00 | 3.70 | 8.75 | 3E+06 | STM2409 | | | | |
| 10.45 | 2.23 | 1.34 | 4.06 | 3E+06 | IR | acrD | RND | + | TTTCGTGCTGATACGTCGCCGCTTCCC |
| | | | | | STM2481- | | family, | | GCTGAAGCCGCGCCCGAAATAAGATCC |
| | | | | | STM2482 | | aminoglycoside/ | | CGGCCAGCCTGATACGAGGTGTCGGG |
| | | | | | | | multidrug | | CACAAAAAAGGCGACTTTCGTTGAGTC |
| | | | | | | | efflux | | GCCTTTTCTTATCCCCTATGGGAGCGC |
| | | | | | | | pump | | GGTGCCTTCCAGGCATTTATTTACGAA |
| | | | | | | | | | GCATGACTTCGATAAAATCTTTCCAGTT |
| | | | | | | | | | CCCCAGTTCACGTTCAATCATAATAGC |
| | | | | | | | | | CTCTCTTATTATTATGGGTATTCTACGT |
| | | | | | | | | | AGTTAGCGGTATAGAGAGAAGTTCATT |
| | | | | | | | | | TAACCGATTGTTGCGATATCCTCTGGTT |
| | | | | | | | | | AT |
| 4.94 | 5.33 | 3.12 | 6.24 | 3E+06 | IR | yfgB | putative | − | ATTTTTGTTTCTTTGTTAGGAACTACCG |
| | | | | | STM2525- | | Fe—S- | | GGGTACTGCTTTCAGGTGTGACAATTT |
| | | | | | STM2526 | | cluster | | GTTCAGACATATGCTATTCCGGCCTCG |
| | | | | | | | redox | | TTATTACACGTTATGGCCCCTGGAGGG |
| | | | | | | | enzyme | | TTGAAAAAAGAAACGCCCCGGTAAGCT |
| | | | | | | | | | TACTGCTCGTCCGGGGGCGCTGCATT |
| | | | | | | | | | GTACAAATTCTGGCGTAAGGATGCCAC |
| | | | | | | | | | GTCTGCACGCGGCATTAGCAAAAATAA |
| | | | | | | | | | TATTTGAACCGATAATTTATCGCCAACG |
| | | | | | | | | | CATTTACAGCGTGAAAGACGAAGGAGA |
| | | | | | | | | | TTAACGGGTGCGCGGGCACACTTCGC |
| | | | | | | | | | CTTC |
| 5.95 | 5.20 | 2.67 | 6.90 | 3E+06 | STM2526 | | | | |
| 9.22 | 2.69 | 1.21 | 5.94 | 3E+06 | IR | glyA | serine | − | ATTCTTCGATAACAGGTCTTGACAAAG |
| | | | | | STM2555- | | hydroxymethyltransferase | | GTTTTTACGCAAACGATTACCTATGCGT |
| | | | | | STM2556 | | | | CAGATAAGGGTTTCCTGAACGAGAGTC |
| | | | | | | | | | TGACGAATTTCAACGGATTTCTTTTCAG |
| | | | | | | | | | CTTTGTGATGCAGATTTTTCACGTTGTT |
| | | | | | | | | | ACCTCCATAACGTAAAGCAGAGAAGAT |
| | | | | | | | | | CCATTTACAATGCAAGGGTATTTTTATA |
| | | | | | | | | | AGATGCATTTGATATACATCATTAGATT |
| | | | | | | | | | TTCACATAAAGGAAGCACGTATGCTTG |
| | | | | | | | | | ACGCACAAACCATCGCTACAGTAAAGG |
| | | | | | | | | | CCACCATTCCCCTGCTGGTTGAAACA |
| 8.94 | 2.69 | 1.33 | 6.15 | 3E+06 | STM2556 | | | | |
| 2.71 | 2.57 | 0.72 | 2.90 | 3E+06 | IR | lepA | GTP- | − | TCTATACGATCTATAAACCTATAAACAC |
| | | | | | STM2583- | | binding | | GGTTACAGTCAGTCCTGACTAAACAGC |
| | | | | | STM2584 | | elongation | | AGCCGGCCTACCGCAGTCACGTTCTTG |
| | | | | | | | factor | | CAGACAACGTGACTGCGGTAATCCATC |
| | | | | | | | | | CCACCGGATTGTCTTCAAATTCTCCATG |
| | | | | | | | | | TTGCTGAATCGGCTAACAGCTTCTTAAA |
| | | | | | | | | | CGATCGGTATTAGGCTAGGTTCTAAAT |
| | | | | | | | | | CTTGCCTGAATGAAAATAAATGTAATAA |
| | | | | | | | | | TGATAGCTTGGTATTGACATATAGATTG |
| | | | | | | | | | AAAAAGCGCATGAAAATAGGATTCCAA |
| | | | | | | | | | CCAGCCATATTGCAATATGCATATAC |
| 2.68 | 2.44 | 0.60 | 2.97 | 3E+06 | STM2584 | | | | |
| 4.64 | 4.54 | 0.35 | 9.55 | 3E+06 | IR | STM2620 | Gifsy-1 | − | GAGTTGTAATTCGTGCGCCATGGTATT |
| | | | | | STM2620- | | prophage | | CTCCGTGGCGCATAATTGTCAGGTTAC |
| | | | | | STM2621 | | | | TGGTTGTTCAGGCCAGTGCGATAATTA |
| | | | | | | | | | TGATTGCGTGCTTATTGTTAAGTCAATT |
| | | | | | | | | | ATTAGAGCCCATCTCTCTGACAACTTCC |
| | | | | | | | | | ATAATGGTATCCTTAGACCAGTACAAAT |
| | | | | | | | | | CATCTCTTGATAGCTTTCGGTTTGTTAC |
| | | | | | | | | | AGAACCGTTTTGGGTTGATAGGATATA |
| | | | | | | | | | GCGATCACCTGATGCAAGTCTAAAAGA |
| | | | | | | | | | CAAAAGTTCTATTCCCTTAGCATCAGTT |
| | | | | | | | | | ATAGTCACCCGCAAATTATCAGCAAG |
| 15.54 | 2.48 | 3.54 | 0.65 | 3E+06 | STM2640 | | | | |
| 19.02 | 2.48 | 2.07 | 4.04 | 3E+06 | IR | rpoE | sigma E | − | ACGCACTATCTGTACAGAAATGCCCAT |
| | | | | | STM2640- | | (sigma 24) | | TTCGTCGTTTGCAGAGTAACCTAACAG |
| | | | | | STM2641 | | factor of | | CATCTTTATTTCACTACAAAATCCGACG |
| | | | | | | | RNA | | CTAACACCCTGCCCTATAAAATATTTTT |
| | | | | | | | polymerase, | | TGCCGTTTATCTCTCGCCGTATTTTTAT |
| | | | | | | | response | | TTTATGTTTAATAAGCACAACACCAGCG |
| | | | | | | | to | | AAATCATAACGTGCTTTTTAGCGCCATA |
| | | | | | | | periplasmic | | TAGTGCTAATCTGCCGCAACCATGTTTA |
| | | | | | | | stress | | GTAAATTAAACAAGAACCATGATGACAA |
| | | | | | | | | | CTCCTGAACTGTCCTGTGATGTGTTAAT |
| | | | | | | | | | TATCGGCAGCGGCGCGGCCGGAC |
| 24.48 | 3.33 | 2.75 | 0.49 | 3E+06 | STM2641 | | | | |
| 2.86 | 3.90 | 1.67 | 13.85 | 3E+06 | STM2659 | | | | |
| 9.64 | 5.65 | 5.87 | 7.55 | 3E+06 | IR | rrsG | 16S rRNA | − | AACGAAGCTTTTCTGACCCGGCGGCCT |
| | | | | | STM2659- | | | | GTATGCCGTTGTTCCGTGTCAGTGGTG |
| | | | | | STM2660 | | | | GCGCATTATAGGGAGTTATTAGAGCCT |
| | | | | | | | | | GACAAGACCTAAATGCAAAAAAAAGCT |
| | | | | | | | | | CAACCGTTCACTTTTCAAACAACATTTG |
| | | | | | | | | | AACCAAAAGCCTATTTTCGCCTGGTTTT |
| | | | | | | | | | TAAACAAAAACGAGCCCGTCAGGGCCC |
| | | | | | | | | | GTTTTATTCAAATTTGTGACTTACTGCA |
| | | | | | | | | | CTGCCACAATACGATCATCATTGGCTT |
| | | | | | | | | | CAAGGCGAATCACTTTGCCAGGAACCA |
| | | | | | | | | | GTTCACCAGACAGGATTTGCTGCGCCAG |
| 19.87 | 1.84 | 2.99 | 2.17 | 3E+06 | STM2662 | | | | |
| 4.23 | 6.25 | 3.58 | 7.92 | 3E+06 | IR | rluD | pseudouridine | − | TTGACCAACACGCGCTGATTCAAAATC |
| | | | | | STM2662- | | synthase | | CATTCTTTTATACGCGAACGTGAATAAT |
| | | | | | STM2663 | | (pseudouridines | | CCGGGAACATTTCGGCCAAAGCCTGAT |
| | | | | | | | 1911, | | CTAAGCGTTGACCGAGTTGGTTTTCGG |
| | | | | | | | 1915, 1917 | | AGACCGTTGCGGTGAGTTGTACTCGTT |
| | | | | | | | in 23S | | GTGCCATATACAGCTTCTTCGTTTAACG |
| | | | | | | | RNA) | | TTGGGTTTTACGGCTTTGCCGTTTAATA |
| | | | | | | | | | TAGTGTGCTATTGTAGCTGGTCTTAACC |
| | | | | | | | | | GGGAGCAGGAACAGAGAATCTCCCGT |
| | | | | | | | | | AAAACATTTTGAGGAAAGTCAAAACGTC |
| | | | | | | | | | ATGACGCGCATGAAATATCTGGTGGCA |
| 4.14 | 3.10 | 1.03 | 4.32 | 3E+06 | STM2663 | | | | |
| 7.50 | 1.89 | 3.23 | 2.75 | 3E+06 | STM2801 | | | | |
| 12.46 | 5.53 | 4.30 | 4.62 | 3E+06 | IR | ygaC | putative | − | ACGGTAAACCCTGCCTTTTCCAGTACC |
| | | | | | STM2801- | | cytoplasmic | | CGCGCCACCTCGTCAGGTCGTAAATAC |
| | | | | | STM2802 | | protein | | ATATTTTATCCTCATTCTCTTGTACTGC |
| | | | | | | | | | GGGCTTACCTTACCCGATAGCGCGTTA |
| | | | | | | | | | TCAACGCTTTCAGAAAAGTCCAGAAAC |
| | | | | | | | | | GCATGATATCGCCGTAACAAGCCTCAG |
| | | | | | | | | | CAGGTAAAAATATGAACTACACTGAAA |
| | | | | | | | | | GCTACATCGAAATCAATGGAGGATCAT |
| | | | | | | | | | ATGCTTAACAAACCGAACCGAAACGAC |
| | | | | | | | | | GTCGATGATGGTGTTCAGGATATTCAG |
| | | | | | | | | | AATGATGTCAATCGATTAGCCGACAGT |
| | | | | | | | | | CTG |
| 13.01 | 4.82 | 4.47 | 4.62 | 3E+06 | STM2802 | | | | |
| 4.25 | 6.94 | 0.48 | 11.09 | 3E+06 | IR | nrdF | ribonucleoside- | + | TCCCATGCCTTTATTTCAAGCAATAGGG |
| | | | | | STM2808- | | diphosphatide | | AGTCAAATCGCGCAAATATTACAACATG |
| | | | | | STM2809 | | reductase | | TCCTACACTCAATACGAGTGACATTATT |
| | | | | | | | 2, beta | | CACCTGGATTCCCCCAATTCAGGTGGA |
| | | | | | | | subunit | | TTTTTGCTGGTTGTTCCAAAAAATATCT |
| | | | | | | | | | CTTCCTCCCCATTCGCGTTCAGCCCTT |
| | | | | | | | | | ATATCATGGGAAATCACAGCCGATAGC |
| | | | | | | | | | ACCTCGCAATATTCATGCCAGAAGCAA |
| | | | | | | | | | ATTCAGGGTTGTCTCAGATTCTGAGTAT |
| | | | | | | | | | GTTAGGGTAGAAAAAGGTAACTATTTCT |
| | | | | | | | | | ATCAGGTAACATATCGACATAAGTA |
| 9.87 | 4.43 | 3.25 | 7.89 | 3E+06 | IR | prgH | cell | − | TGTATAATGCGTCTCAACACATATTAAA |
| | | | | | STM2874- | | invasion | | AGAACCATCATCCCCATTGGGGCTTAA |
| | | | | | STM2875 | | protein | | ACTACTGTAGATAAATTACCCAAATTTG |
| | | | | | | | | | GGTTCTTTTGGTGTAACAATCAGACCAT |
| | | | | | | | | | TGCCAACACACGCTAATAAAGAGCATT |
| | | | | | | | | | TACAACTCAGATTTTTTCAGTAGGATAC |
| | | | | | | | | | CAGTAAGGAACATTAAAATAACATCAAC |
| | | | | | | | | | AAAGGGATAATATGGAAAATGTAACCTT |
| | | | | | | | | | TGTAAGTAATAGTCATCAGCGTCCTGC |
| | | | | | | | | | CGCAGATAACTTACAGAAATTAAAATCA |
| | | | | | | | | | CTTTTGACAAATACCCGGCAGCAA |
| 9.87 | 4.47 | 3.25 | 8.16 | 3E+06 | STM2875 | | | | |
| 3.68 | 4.26 | 0.55 | 5.31 | 3E+06 | IR | STM2903 | putative | − | GGTTGTGTCCCTATTACGCGGGTAGGA |
| | | | | | STM2903- | | cytoplasmic | | TCAATCAAGCAGTTACGGCAAAAAAGA |
| | | | | | STM2904 | | protein | | GAATCATGGATATATTTAGCAAACTCCC |
| | | | | | | | | | TGATGATACGTAATCAGTGAGATTAAAA |
| | | | | | | | | | TAATGCAATCGCGATAAACCGAAGTTA |
| | | | | | | | | | ATCCCCTGTTTAAAGACAGTGAGCGAC |
| | | | | | | | | | CTTCTTGCCATGCCTGGACTATATCAG |
| | | | | | | | | | CCTCATATGTACGCCTTGAAAGCGTAC |
| | | | | | | | | | AGATATGTATTATAATTGTACATATTGTT |
| | | | | | | | | | CATAAACAGGAGGATGAAAACCATGCC |
| | | | | | | | | | TCAGATAGCTATAGAATCTAACGAAAG |
| 3.81 | 2.82 | 0.55 | 5.19 | 3E+06 | STM2904 | | | | |
| 4.30 | 2.81 | 0.47 | 5.50 | 3E+06 | STM2954 | | | | |
| 3.43 | 3.95 | 0.42 | 4.50 | 3E+06 | IR | mazG | putative | − | ACTTCATAGGTTTCTTCCAGCGTATAAG |
| | | | | | STM2954- | | pyrophosphatase | | GCGCGATGCTGGCGAAGGTCTGCTCTT |
| | | | | | STM2954.1n | | | | TATCCCACGGGCAGCCGTTTTCCGGGT |
| | | | | | | | | | CGCGCAGGCGCTGCATGAGGGTGAGA |
| | | | | | | | | | AGACGGTCAATTTGATGGTTAGTTGTC |
| | | | | | | | | | ATGGTTTTTAATCGGTTGTAAATACCAG |
| | | | | | | | | | CGACAATTGTAACGTATTATTCTTAACC |
| | | | | | | | | | ATTCACGCACAGAGACACTACGACAAC |
| | | | | | | | | | GCCTATATAATAAAATATATTGTTAACA |
| | | | | | | | | | GGTGTTGAATGCTACCTTTCCCGTATAA |
| | | | | | | | | | CTTTAAAATTATTAATCGATACACAAC |
| 10.45 | 4.17 | 2.04 | 7.90 | 3E+06 | IR | araE | MFS | − | AATGGCTACGCTATAGCGATATGTGAT |
| | | | | | STM3016- | | family, L- | | GGATATTACACTTTTTAAATTTAACGCC |
| | | | | | STM3017 | | arabinose: | | GTTGCCGGGTATTTTTTTAAACCACCAA |
| | | | | | | | proton | | TATTTCAATGAATTAAAGCATTGATCAT |
| | | | | | | | symport | | AGCTATTATTTAACAATATATGGATTAA |
| | | | | | | | protein | | GTTAAACCCACAATATGGACTATGCTAA |
| | | | | | | | (low-affinity | | TGAGATCATAAAAAAACCCTGTACGAG |
| | | | | | | | transporter) | | GACAGGGCTTTATCAGTTTTTTCGGCC |
| | | | | | | | | | AAAGCGTCGATTTTCCCAGAAACGCAT |
| | | | | | | | | | TTGTCAGTAGCGGATTAACGCGCCAGC |
| | | | | | | | | | CAACCGCCATCTACCGCTATGGTATA |
| 9.65 | 4.43 | 2.52 | 14.23 | 3E+06 | STM3017 | | | | |
| 2.67 | 2.05 | 2.00 | 6.06 | 3E+06 | STM3023 | | | | |
| 3.43 | 1.93 | 2.11 | 6.54 | 3E+06 | IR | yohL | putative | − | TGTAACACGGCCGCGCATTCATGCGGT |
| | | | | | STM3023- | | cytoplasmic | | TCATCCAGCATTTTTTTTAGCGCTATCA |
| | | | | | STM3024 | | protein | | CCTGTCCCTGAATCTTGCTGGTTCTGG |
| | | | | | | | | | CTTTAAGCTTTTGTTTGTCCCGGATGGT |
| | | | | | | | | | ATGTGACATTACAACACCTCACTAACAT |
| | | | | | | | | | TAACGAATACAAATTATAGCATTACGAT |
| | | | | | | | | | GCTACTGGGGGGTAGTATTCTATACTG |
| | | | | | | | | | GGGGGGAGTAGAATGACGCCCACATA |
| | | | | | | | | | AAACAACTAAGAATCATTCTCATGGGTG |
| | | | | | | | | | AATTTTCGACACTTCTTCAGCAAGGAAA |
| | | | | | | | | | CGGCTGGTTCTTCATTCCCAGCGCCA |
| 3.14 | 1.93 | 2.06 | 7.47 | 3E+06 | STM3024 | | | | |
| 3.46 | 3.76 | 1.45 | 6.82 | 3E+06 | STM3059 | | | | |
| 3.46 | 4.12 | 1.38 | 6.74 | 3E+06 | IR | ygfB | putative | − | ATGAGCTGTCGTTGTTGCCGCCGCAAA |
| | | | | | STM3059. | | cytoplasmic | | TCATCCCGCTGATTAAACCATGCATTTC |
| | | | | | S- | | protein | | AGCCGGGGTCAGACCGGCCCCTTGTT |
| | | | | | STM3060 | | | | GATTCAAAAACCGGTTCATTTCGTTGTA |
| | | | | | | | | | ACCAGGCATTTCGTTCTGTATAGACATA |
| | | | | | | | | | AGCATTCGTCATCAAAGGGAGGATATT |
| | | | | | | | | | CATGATATGCTACCACTTTGGACCCTG |
| | | | | | | | | | GTGAACCAGAAAAGGGCTTGTATCTTC |
| | | | | | | | | | ACACCAGGGTAGCTATAGTGTCGCCCC |
| | | | | | | | | | TTCGCGGACCCTGGGTCTGGAGACGA |
| | | | | | | | | | AGGCAGCGCAGTCAATCAGCAGGAAG |
| | | | | | | | | | GTGG |
| 8.64 | 3.59 | 3.25 | 2.57 | 3E+06 | STM3060 | | | | |
| 10.29 | 5.01 | 3.53 | 9.98 | 3E+06 | IR | serA | D-3- | − | CTTTTTTGCCATCTGATGTTGTGTGTGG |
| | | | | | STM3062- | | phosphoglycerate | | ATTTGCATCCGTCCTTCAACATATCAAA |
| | | | | | STM3063 | | dehydrogenase | | AAAAATTATCACGGCAATATGAACGTTT |
| | | | | | | | | | GCGCCAGCGTCGTGAAGGAATCGCAT |
| | | | | | | | | | ACAGCGGGAAATAGCAGATGAAAATAC |
| | | | | | | | | | CGGGAATAACTTTTTCTTTGGAGGGAT |
| | | | | | | | | | CGGCAGGGCAAACGATTAAACGTGATA |
| | | | | | | | | | CATGTCACCAAATTTGCCCTGACCGAA |
| | | | | | | | | | TTTTTTACGCGGCAGGAAATACGCCTG |
| | | | | | | | | | GCGGGATCATTTTACGATGGTTTTCAC |
| | | | | | | | | | CCCGTCCGGCGTGCCGATCAGTGCGA |
| | | | | | | | | | CAT |
| 10.25 | 4.50 | 3.68 | 9.08 | 3E+06 | STM3063 | | | | |
| 8.70 | 6.90 | 4.94 | 2.66 | 3E+06 | STM3083 | STM3083 | putative | − | |
| | | | | | | | Mannitol | | |
| | | | | | | | dehydrogenase | | |
| 6.87 | 6.27 | 5.83 | 3.36 | 3E+06 | IR STM3083- | | | | TGAGATCGTTATAAACAGCCTGATGAC |
| | | | | | STM3084.S | | | | CACGGTGAAAGGCGCCAAATCCAATAT |
| | | | | | | | | | GTACGATGTTGGCTTCCATTCCCTGAC |
| | | | | | | | | | GTGAATAAGTCGTTTTGAATTGGTGCCT |
| | | | | | | | | | TGCGGCGTCTAACTGGCGAGCTATGGT |
| | | | | | | | | | GTCCATGAATTTTTCCCACTCCTGTTTT |
| | | | | | | | | | GTTTACCAATTCTGCTTAAACACCATAC |
| | | | | | | | | | CAAAATCCGTGAATATGATCACACTCAT |
| | | | | | | | | | GGCACCAGATTCTTTACCATGGTATGC |
| | | | | | | | | | TGACTAATAGCCAATGAATAAAAATAAT |
| | | | | | | | | | TTATTTATCAATTAGTTATAAAAAGC |
| 8.91 | 3.97 | 0.22 | 11.50 | 3E+06 | IR STM3168- | ygiR | putative | − | TGTTTGAAATTGGTCTTATGAATATCTT |
| | | | | | STM3169 | | Fe—S | | CAAATTGGTATGCAATTAATTATACCCA |
| | | | | | | | oxidoreductase | | CGTCTAAAAACGCAGTATCGTCATAAC |
| | | | | | | | family 2 | | AACAAAAAGTAAAAAAACATCACATTAT |
| | | | | | | | | | CAGTAATATATAAAAAAACTTCGCTGAA |
| | | | | | | | | | TTGCTCACGACACTGTTTTTACCATGAC |
| | | | | | | | | | TTTCTTCTGTGAACCAGATCTCTTTCTT |
| | | | | | | | | | TGGTCTATTGATTAAATTAAATTGGCTG |
| | | | | | | | | | ACAGAATTCAGGGGATAAAGAACACCA |
| | | | | | | | | | TCACCACGCCTTTCCCCAACGCAACAC |
| | | | | | | | | | CTTACGTATCAGCAGGTTATTAAT |
| 8.70 | 5.18 | 1.38 | 13.67 | 3E+06 | STM3169 | | | | |
| 4.81 | 2.12 | 0.39 | 3.00 | 3E+06 | IR STM3195- | ribB | 3,4 | − | TCCGGACTTTAACCGTCGGCCCCGGAA |
| | | | | | STM3196 | | dihydroxy- | | TTACACCGGATCTGCTGACCTTTTCGC |
| | | | | | | | 2- | | TATGGCAAAAAGCGCTCGCGGGCTTTC |
| | | | | | | | butanone- | | AACCTGCTCTCCGCGTTCCGTCACGGC |
| | | | | | | | 4- | | GCGCCGTGATGAGAAATGCGTTAAACA |
| | | | | | | | phosphate | | TCGCTGATTTACCGCCGGTGGGGAATT |
| | | | | | | | synthase | | TCGCCCCGCCCTGAGAATAAGCGGGTT |
| | | | | | | | | | AACTATAACGCTATTGATTACCTTCATC |
| | | | | | | | | | AACGCCTTTACTCCGTATGACGTCACA |
| | | | | | | | | | CAATTCTGGTTTATGGCGTCCACATATC |
| | | | | | | | | | GCACTACAATAAGAGCTAACACTTACC |
| | | | | | | | | | AG |
| 4.57 | 2.33 | 0.38 | 3.20 | 3E+06 | STM3196 | | | | |
| 4.31 | 3.54 | 1.26 | 4.72 | 3E+06 | STM3202 | | | | |
| 4.70 | 3.24 | 1.03 | 5.13 | 3E+06 | IR STM3202- | ygiF | putative | − | GTTATCAGGCGTTTCGAAGTAGATATTC |
| | | | | | STM3203 | | cytoplasmic | | AGCAACTGGCTGGGCGCATGATGCTC |
| | | | | | | | protein | | GCCGCCGAGCGTATGAAGATGATTTCG |
| | | | | | | | | | CAGCGCATCTACGGCGTCGTGATTGAC |
| | | | | | | | | | GATAAACTTTAATTCGATTTCCTGAGCC |
| | | | | | | | | | ATGGCCTTGTACTTATGGGTTATGTCAC |
| | | | | | | | | | ATCTGGGAAGATTCTTGGCGAACTTAC |
| | | | | | | | | | CCGCATTATTTTTGTCAGTAGATAGTAT |
| | | | | | | | | | TTTGCGCCAAATTGCCATGCAACGAGC |
| | | | | | | | | | AATTTGACGGGCGTAAAAGTTTGACGT |
| | | | | | | | | | AGCGGCAAAGGCGACACAGATGATTCCG |
| 4.20 | 4.68 | 1.34 | 5.12 | 3E+06 | STM3203 | | | | |
| 2.91 | 2.54 | 2.85 | 2.95 | 3E+06 | STM3214 | | | | |
| 4.36 | 2.62 | 4.77 | 2.91 | 3E+06 | IR STM3214- | yqjH | putative | − | CCCGCAGAACGATCAGCTCGCGAAAAC |
| | | | | | STM3215 | | transporter | | GCAGCTCATTACGAACACGCTGTGGGT |
| | | | | | | | | | AGCGTACGGATGATGTCGTCATTTTTT |
| | | | | | | | | | GCCTTCGTGAAGTAATACGATATATCTA |
| | | | | | | | | | AATTAAAGTTTTAAATGATAATGATTGTT |
| | | | | | | | | | AATCAGTAAAAATGCAACTGTTTTTTGA |
| | | | | | | | | | TAGTGTTCTGGCAACACATCGCTAATC |
| | | | | | | | | | ACAACTTCAAAATAAAACGTTATAAATT |
| | | | | | | | | | AATAGATTATATCAACAATCGCTTTTAT |
| | | | | | | | | | CCTTGCTAAAAACCATCATTTAGATATA |
| | | | | | | | | | AATTAGATATATCTAAATAAGCAG |
| 3.38 | 1.90 | 3.56 | 2.09 | 3E+06 | STM3215 | | | | |
| 16.37 | 5.99 | 0.24 | 12.63 | 3E+06 | STM3245 | | | | |
| 12.29 | 5.70 | 0.27 | 9.88 | 3E+06 | IR STM3245- | tdcA | transcriptional | − | AAAATAGGCCTCAACATCGCTAATGATT |
| | | | | | STM3246 | | activator of | | TTACTGACGGCGGGTTGGGTTAACCCT |
| | | | | | | | tdc operon | | AACGATTTTGCGGCAGAACCGATAGAA |
| | | | | | | | (LysR | | CCACTTCTAATGACTTCCTGAAAGACCA |
| | | | | | | | family) | | CCAAATGCTGTGTTTTAGGGAGAACAA |
| | | | | | | | | | GAGTATTCATATCTACCGCTCTGAAATA |
| | | | | | | | | | ACATTGTGAACGGCAGGAAGTGTAGCA |
| | | | | | | | | | AATTAAATCTTAAAGGTTATGTGCGACC |
| | | | | | | | | | ACTCACAAATTAACTTACCACAATTTTT |
| | | | | | | | | | ACATGGTTTTTATTAAATAAAGAAAACC |
| | | | | | | | | | TGATATTTCAATAGGTTACAAAAAT |
| 2.46 | 4.21 | 0.82 | 4.51 | 3E+06 | STM3297 | | | | |
| 2.33 | 5.69 | 1.36 | 8.16 | 3E+06 | IR STM3297- | ftsJ | 23S rRNA | − | CAAGTTTAAACCAGGCACGGGAGCGTA |
| | | | | | STM3298 | | methyltransferase | | GCCCCTTTTTCTGCGCCTGTTGAACAT |
| | | | | | | | | | ATTTATCGCTAAAGTGTTCCTGAAGCCA |
| | | | | | | | | | GCGGCTTGAGCTGGCAGAACGCTTTTT |
| | | | | | | | | | ACCTGTCATTTAACTTTCCCGTCGGGG |
| | | | | | | | | | CAGTTCATCGTAGCCAATGGCGTAAAT |
| | | | | | | | | | TTCTACACGCCTATTTGGCGATATAAG |
| | | | | | | | | | GGAGATGGCGGTAGAATGACCCGTTTT |
| | | | | | | | | | CAATCCCAACGTAAGCAAAAATATACG |
| | | | | | | | | | ATGAATCTGAGTACTAAACAAAAACAGC |
| | | | | | | | | | ACCTAAAAGGTCTGGCACATCCGCTCA |
| | | | | | | | | | AG |
| 2.78 | 5.49 | 1.44 | 9.14 | 3E+06 | STM3298 | | | | |
| 8.69 | 3.03 | 0.58 | 9.26 | 4E+06 | IR STM3342- | sspA | stringent | − | GACCAGAAAACAGCGTCATTACCGAAC |
| | | | | | STM3343 | | starvation | | GTTTGTTGGCAGCGACAGCCATGAAAA |
| | | | | | | | protein A, | | CCTCCAGGTATATTCAGAATTTTTACTG |
| | | | | | | | regulator of | | CTACCAGCCACAATGTGACCAGCCAGA |
| | | | | | | | transcription | | TGTTATGTCACCCAGGGCGAAAAAAGC |
| | | | | | | | | | CATCATTGCTCAGAAACGAGACAAAAA |
| | | | | | | | | | ATGAACATTCCCCGCTATTTGGGCAGA |
| | | | | | | | | | AAATTGGATGATAGTTTACCAGATTTTG |
| | | | | | | | | | TGACCTTTGTGGTGAGTCGATTCTGGA |
| | | | | | | | | | AATGAGGAAAAAGAGATATTCCTGGTC |
| | | | | | | | | | TGAAATGCTCGCCCCACCTGAGATATT |
| | | | | | | | | | GT |
| 7.68 | 2.23 | 2.54 | 7.89 | 4E+06 | STM3343 | | | | |
| 2.34 | 1.09 | 10.63 | 3.05 | 4E+06 | STM3356 | | | | |
| 3.75 | 1.53 | 6.02 | 2.87 | 4E+06 | IR STM3356- | STM3356 | putative | − | CATATTTATAATTATCCAATCAATGATAT |
| | | | | | STM3357 | | cation | | ATGATATTGTATCCAATGTTGGCAGGG |
| | | | | | | | transporter | | AGAAATTATTCCCATACAAAAACTAAGT |
| | | | | | | | | | CAAATCGTTTCTCAGGAAAGATGCAGG |
| | | | | | | | | | AGTGGGATCTACATCAAGATCGTGGTT |
| | | | | | | | | | AGATCGTTACTGGACGTGATTAATAGA |
| | | | | | | | | | ATTGAAGAATTGGTTGAAGCGCCTGCG |
| | | | | | | | | | ATGCTCACGCAGGCGAAAAGATCAGGC |
| | | | | | | | | | AGAAGGGTCACCAACATAGCGGGTCA |
| | | | | | | | | | GCATATTCTCCATTGAGCGAATAATGTG |
| | | | | | | | | | TTCGCGCATGCGCTGGCGTGCCAATGTT |
| 4.71 | 2.01 | 3.72 | 1.67 | 4E+06 | STM3357 | | | | |
| 5.39 | 3.55 | 0.98 | 5.58 | 4E+06 | STM3378 | | | | |
| 4.65 | 3.71 | 2.07 | 8.91 | 4E+06 | IR STM3378- | STM3378 | putative | + | TAGCCCTTTTAGCGTTGCGTTACCGGA |
| | | | | | STM3379 | | inner | | AGTTTCGCCAGTGGTGGCGCTAGTTTG |
| | | | | | | | membrane | | GTGAACTGTGCGGTCGATTGCAAAACG |
| | | | | | | | protein | | CAAAACAGGTAATGTCCTTTTTATGTTT |
| | | | | | | | | | CGGGTTGATTATCTTCCCTGATAAGAC |
| | | | | | | | | | CAGTATTTAGCTGCCAATTGCGACGAA |
| | | | | | | | | | ATAGTTATAATGTGCGACTTTACATTGC |
| | | | | | | | | | CCAACGGCGATTTTCGTTCGCAGAAAG |
| | | | | | | | | | GGTGACAATCGAGCAATGAAGGTATAT |
| | | | | | | | | | TTTGTTTTTTGCCCGAAAATGGCAGAAG |
| | | | | | | | | | ATAGCCACACAATGACTGGCAAATCATG |
| 8.32 | 6.32 | 2.17 | 10.71 | 4E+06 | STM3405 | | | | |
| 7.92 | 4.90 | 2.30 | 8.48 | 4E+06 | IR STM3405- | smf | putative | − | GTTCAGCTTGCCGCGCGGTAAGACCA |
| | | | | | STM3406 | | protein | | GCCTCCTGAAGGTGCGTGCGATTTATC |
| | | | | | | | involved in | | TGAGGCTGGCGAATAAGCGAGTTCGC |
| | | | | | | | DNA | | CATGTTCAACATCGCCTCGCCATAAAG |
| | | | | | | | uptake | | GTCGCCGACGTACATTAAACGTAACCA |
| | | | | | | | | | AATTTCGGTACGGGCCATCCTTTCCCT |
| | | | | | | | | | CCCCTGCCACAAGCAGTCTGAACAATC |
| | | | | | | | | | TTTGCGATTGGTCACTGATGCTGTCAAT |
| | | | | | | | | | CAGGTGGGGATTTGTCTAGAATAGAGG |
| | | | | | | | | | TAATAATCTTTTCAACTCCTGAACACAA |
| | | | | | | | | | CTCTGGATAATTATGTCAGTTTTGCAAG |
| | | | | | | | | | TGT |
| 13.47 | 1.74 | 3.60 | 2.98 | 4E+06 | IR STM3453- | fkpA | FKBP-type | − | GATTTCATCCATATCTCCAGGGCCGGG |
| | | | | | STM3454 | | peptidyl- | | GCATCTCGCCCCATGTTAACTTACGTA |
| | | | | | | | prolyl cis- | | AGAAGCGTACTATAAATCGTTGCAGAA |
| | | | | | | | trans | | CAAATCAACATACGAACACGCCCTATTA |
| | | | | | | | isomerase | | TCACTTCTTTTCAGACTCTTTTTGTTTAA |
| | | | | | | | (rotamase) | | ATTAGTTTCGTAGTGCGCGTAATGGTT |
| | | | | | | | | | GCTGTGAAAGCCGGTAAAGTTAAGTAG |
| | | | | | | | | | AATCCGCCGACGGAGACAACATAAAGA |
| | | | | | | | | | GGTACATCATGCAGGATATCACGATGG |
| | | | | | | | | | AAGCTCGTCTGGCTGAACTGGAAAGCC |
| | | | | | | | | | GTCTGGCGTTCCAGGAGATTACCATAGA |
| 12.79 | 2.04 | 3.73 | 3.72 | 4E+06 | STM3454 | | | | |
| 14.28 | 4.61 | 0.55 | 10.24 | 4E+06 | STM3487 | | | | |
| 10.28 | 7.90 | 2.02 | 12.47 | 4E+06 | IR STM3487- | aroK | shikimate | − | AAAGATATTGCGTTTCTCTGCCATTTTT |
| | | | | | STM3488 | | kinase I | | TCGGTACTACTAAGACTATTCGTTAATG |
| | | | | | | | | | GTAAACCCGCTTCACAGACACCCAGCG |
| | | | | | | | | | CAGCAGGACATGAACTGAAACCTCATA |
| | | | | | | | | | AGATATTGCGAGAGTCAGACTGAAAAT |
| | | | | | | | | | TATCTCAATACTCAAGCGGGTTTGGCA |
| | | | | | | | | | ACTGAATAAATCACCAAGCCTGATTGTT |
| | | | | | | | | | GCAAAACCCGAGTTAGCGTTGCCGAAT |
| | | | | | | | | | GGCGACCAGAACAACATATCCGGCCTA |
| | | | | | | | | | CAAATTGCTCTACTTTCAAACAATTGTG |
| | | | | | | | | | CGCAATCCGCAGAACCAATACGTCTGC |
| 11.79 | 2.63 | 1.44 | 3.45 | 4E+06 | IR | yrfE | putative | − | CACGCGACGCACGCCGTTGCTGAACT |
| | | | | | STM3494.S- | | NTP | | CCAGATCCACGCTTTCTACGTTAAACA |
| | | | | | STM3495 | | pyrophosphohydrolase | | GTCGGGATTGTGCGACGGTTTCCACTT |
| | | | | | | | | | TCAGAATGGTGGGTTTTTGTAATGATTT |
| | | | | | | | | | GCTCATTGTGAGAATCTTTGCAGTGTAA |
| | | | | | | | | | TCTGTGGTCATTGTGCGACATACCGCA |
| | | | | | | | | | CGGTTTCGGCAATGCGAATTGCCGTTT |
| | | | | | | | | | ATTTACATTTATGTAACGTAATAAAAATT |
| | | | | | | | | | AATTCTTATTTCAAATTAAAAGTCAATAG |
| | | | | | | | | | GTTGAAATAACTCCAGGAATTTGCTGAT |
| | | | | | | | | | ATTCCGTTTTTGGTGGTATTGCTAT |
| 10.33 | 4.08 | 0.35 | 3.90 | 4E+06 | STM3495 | | | | |
| 19.41 | 3.10 | 2.01 | 7.35 | 4E+06 | IR STM3504 | yhgF | paral | + | TTAAACATTAAAAACGGTGAATATTTGC |
| | | | | | STM3505 | | putative | | ACATTAGAGGTATTTGCAAAAAGACAAA |
| | | | | | | | RNase R | | TAAATGTTGAGCCATATCAACATCGGC |
| | | | | | | | | | GCAAATTATCGCTTATTTGTACATTCCG |
| | | | | | | | | | TCACATTTTAATCGTTGAAGATAGAAAC |
| | | | | | | | | | CATTCTCATTATCATTGTGTTGTTGATT |
| | | | | | | | | | ATTTACTCTTTCCTTCGTTGGCTAAACA |
| | | | | | | | | | TCGGGTCTCCTGCCGCCCCCCTGAGC |
| | | | | | | | | | GCCGCATGAGGTATACATCCAGTTAGT |
| | | | | | | | | | AAGAAACAAGTAGGTCGTATGCAATTC |
| | | | | | | | | | ACTCCTGACACTGCGTGGAAAATCAC |
| 14.38 | 3.01 | 2.01 | 6.02 | 4E+06 | STM3505 | | | | |
| 8.26 | 3.35 | 6.09 | 4.90 | 4E+06 | STM3511 | | | | |
| 9.21 | 2.28 | 8.65 | 5.12 | 4E+06 | IR STM3511- | yhgI | putative | + | TGGTTGACGTCACGCTGAAAGAAGGGA |
| | | | | | STM3512 | | Thioredoxin- | | TCGAGAAACAGTTGCTGAATGAATTCC |
| | | | | | | | like | | CGGAACTGAAAGGGGTTCGCGATCTGA |
| | | | | | | | proteins | | CCGAACACCAGCGCGGCGAGCACTCA |
| | | | | | | | and | | TACTACTAAGATTTTCCCCGCATCCATG |
| | | | | | | | domain | | CCCGATGGCGCTTGCGCCTGTCGGGC |
| | | | | | | | | | CTTGTCAGCCCCACCGTAGGCCGAATA |
| | | | | | | | | | AGGCGTCTACGCCGCCATCCGGCGCT |
| | | | | | | | | | ATCAACCACATCTCATAACAATGGCCCT |
| | | | | | | | | | TCTTCTTTCGCCGATAACATGACCTGTG |
| | | | | | | | | | TCTCATAATTTAAATTTTGCCTGCCAGG |
| | | | | | | | | | GTC |
| 5.59 | 2.28 | 1.83 | 3.95 | 4E+06 | STM3559 | | | | |
| 10.95 | 2.11 | 2.86 | 7.17 | 4E+06 | IR STM3559- | yhhV | putative | − | CCCACGACGCGTGATGGTAACAGGCC |
| | | | | | STM3560 | | cytoplasmic | | CCCCCGTCACCGCACTTTCCAGGACTT |
| | | | | | | | protein | | CGGCCAGATTTTGCCGCGCTTCGCTAT |
| | | | | | | | | | AGTTAACCGTACGCATAAACATCTCCC |
| | | | | | | | | | CAGTTGTACATGTTTATTGTACAACAAA |
| | | | | | | | | | CATGTACAAAAAAAGAGCCATCAGGCT |
| | | | | | | | | | CTTTTGAAAAATTTTACCGCTTGCCGTT |
| | | | | | | | | | ACCGGGGGCGGCGCACGCGCTTCCCC |
| | | | | | | | | | CCTGGCACAGTCTAACCGCCCAGATAG |
| | | | | | | | | | GCGCTGCGCACCGCTTCGTTCGCCAG |
| | | | | | | | | | CAGTGCATCACCGGTATCGGATAGCAC |
| | | | | | | | | | CACGT |
| 10.33 | 2.11 | 3.06 | 7.27 | 4E+06 | STM3560 | | | | |
| 7.59 | 2.00 | 1.08 | 7.04 | 4E+06 | IR STM3590- | uspB | universal | − | AGACAATCAGTGAAAGAGTACTACGAA |
| | | | | | STM3591 | | stress | | AGCCGTCCATATTAGCGCTCCGCATTC |
| | | | | | | | protein B, | | GAACGGCTCTTATACACATTGTAGGAG |
| | | | | | | | involved in | | ATCAGTTAATTTTTTTACCAGAAGGTTA |
| | | | | | | | stationary- | | ATCACTATCAATGCAATTCCCTAGAAAT |
| | | | | | | | phase | | TTTGTTTAACTAACTGGCAAGCAAGGC |
| | | | | | | | resistance | | AGATTGACGGATTATCCTGGTCGCTAT |
| | | | | | | | to ethanol | | AATGTAAGGATAGTTATGGTAAACGGC |
| | | | | | | | | | TGAGCTAGCCCCGCGCATAGAGTTCGC |
| | | | | | | | | | AGGACGCGGGTGACGCGGCGGCATAA |
| | | | | | | | | | GAAACGCCAGTAGCTCAATGGTCATCG |
| | | | | | | | | | ACA |
| 5.44 | 1.39 | 2.01 | 5.66 | 4E+06 | STM3591 | | | | |
| 5.41 | 2.58 | 2.89 | 4.25 | 4E+06 | IR STM3630- | dppA | ABC | − | TTCAGAAGGGTATTTTCAGCAGGGAAA |
| | | | | | STM3631 | | superfamily | | TTTGTGCTATGGCCAGAAAGGCAGAGT |
| | | | | | | | (peri_perm), | | TATTCACTTAATATTTTGCAACAGTTAG |
| | | | | | | | dipeptide | | TGATTAACAATTAGACATTAATTGAAAA |
| | | | | | | | transport | | ATTTCTTTCGATATGTTGATTATCTGAG |
| | | | | | | | protein | | CGATTAATACCACTAACGCTAAAACGC |
| | | | | | | | | | ACAGGCGAAAATGCTGAGGTTATCCAT |
| | | | | | | | | | AAGCCGTGTGCAAAAAAGAGTTATACG |
| | | | | | | | | | GACGTTGAAAAACACCATCGAATATGT |
| | | | | | | | | | CACAAAATTGTAAATAAGTAGGCCGTC |
| | | | | | | | | | GTGCGGCCTACCGCGATCACAAAAACTA |
| 12.80 | 2.93 | 1.08 | 10.12 | 4E+06 | IR STM3684- | yibF | putative | − | CATTAATAAATTCGAAGGTAATACCCTT |
| | | | | | STM3685 | | glutathione | | TTCGAGCAGCAGAACAGAGATTTTGCG |
| | | | | | | | S- | | CACAAAAGGGCTGGTGTAGCTACCGAT |
| | | | | | | | transferase | | GAGTTTCATGCCGTGTCCTTTTTGCCAA |
| | | | | | | | | | CCAGTAAAAATCATAGTATGGCTCAAAT |
| | | | | | | | | | AAGACGAAAAGAGACACAAAAGGAGGT |
| | | | | | | | | | TGCTGAATGACATAACGTGAGAGGACT |
| | | | | | | | | | CGCGACAAAATGTTTGTCGGATCGTAT |
| | | | | | | | | | TGACGTTACCCGGGCTTAAAATTTCTTG |
| | | | | | | | | | TGAAGAGGATCACAAAAATTCAACAAA |
| | | | | | | | | | GCACCAAAATAAAAATGTGAAATATCT |
| 3.23 | 3.46 | 4.44 | 3.72 | 4E+06 | IR STM3793- | STM3793 | putative | − | TAAAATAACATTATCATGTTACTTCCGT |
| | | | | | STM3794 | | sugar | | ATCATTTGTGACTATGATCGCGATTAGA |
| | | | | | | | kinase, | | GGATCATTTTGCCATTTACTTCGTGAAC |
| | | | | | | | ribokinase | | AATCCCTGGCGGAACATACGCGCACCA |
| | | | | | | | family | | AATCATTTTTATTGTTACAATTTACTGAA |
| | | | | | | | | | AATTAACTATTTATTGTTATAAAACGCG |
| | | | | | | | | | AATAAACCCACTTTTATTTCCTGACAGC |
| | | | | | | | | | CGGACGTATAGTAGTGCCACACTGTAA |
| | | | | | | | | | TGTTCTCAGAAACACATAAATGTTACTG |
| | | | | | | | | | ATGGAACATAACAACATGATTTGCGGA |
| | | | | | | | | | GAGGGTGAATGGAGACCAAGCAA |
| 2.88 | 3.00 | 3.22 | 4.38 | 4E+06 | STM3794 | | | | |
| 25.73 | 6.53 | 7.93 | 10.67 | 4E+06 | IR STM3820- | STM3820 | putative | − | ACCCGGACAAACCTAAATAACATAACA |
| | | | | | STM3821 | | cytochrome c | | GCCCAACGGTGATAACTGTTGTCGCAT |
| | | | | | | | peroxidase | | AGAGGGTAATTTTTTTCATATCACTATC |
| | | | | | | | | | CTTATGGGGTATTGCGGCATGATTAATT |
| | | | | | | | | | AAATTTTATTTTTTTACTCATGAGGCCC |
| | | | | | | | | | GTCAATACTAAATACAAACCCATCATGG |
| | | | | | | | | | ATATTGATTGGTATCAATAATTACAATT |
| | | | | | | | | | GGCTAAACCTATAGATATGATAACCCC |
| | | | | | | | | | CGACTATCGTAAGATTTATTTTGCGATG |
| | | | | | | | | | TCCGTCACAGGGTTTATTCAGCAGCAA |
| | | | | | | | | | CAATGGATAAATCCTCTTTTCCGTC |
| 23.33 | 6.41 | 8.05 | 13.85 | 4E+06 | STM3821 | | | | |
| 7.60 | 3.77 | 4.14 | 0.75 | 4E+06 | STM3857 | | | | |
| 9.06 | 2.97 | 5.72 | 3.09 | 4E+06 | IR STM3857- | pstS | ABC | − | CGATAAGGTCGCGGCGACAACAGTTG |
| | | | | | STM3858 | | superfamily | | CGACAGTGGTACGCATAACTTTCATAAT |
| | | | | | | | (bind_prot), | | GTCTCCTGCACGGTTTCGGTAAATCGT |
| | | | | | | | high-affinity | | TGTTTGAGTTGCTACGATGAGCAAAATA |
| | | | | | | | phosphate | | GGACAAATTGATGACAGTTATATGTCTT |
| | | | | | | | transporter | | GATTATGACGGTTTGATGACAATGGAA |
| | | | | | | | | | ATAAAAAAAGCTGGCCCGGGGAGACAC |
| | | | | | | | | | CAGACCAGCCTGCAGGGGGAGATGAA |
| | | | | | | | | | TTAGACTGTTTGCGCAACCGCAGACGG |
| | | | | | | | | | TTTCAACAGCGCGTACATCAGGCCGCA |
| | | | | | | | | | GACAATCGTGCCCAGGGCAATCGAGA |
| | | | | | | | | | GCAG |
| 9.06 | 2.15 | 5.89 | 3.60 | 4E+06 | STM3858 | | | | |
| 2.26 | 6.29 | 0.46 | 10.23 | 4E+06 | IR STM3899- | yifB | putative | − | TGGCGTCATTTTCAGGTAAGAAACATC |
| | | | | | STM3900 | | magnesium | | AAACTGGAAGAACGCTCGCAGAAGCGA |
| | | | | | | | chelatase, | | AAAGAAGGAAAACAGGATGTAGAGTGC |
| | | | | | | | subunit | | GCCAAAAGGGGGAGGAAAACGTGAAA |
| | | | | | | | Chll | | ATTTTTCAGTTGCTAATTTTTCTTATAAA |
| | | | | | | | | | AAACAAAGTACTTTTAGGCATTCACCTG |
| | | | | | | | | | CATTATCTGAAACGTGGTTAAAAAAATA |
| | | | | | | | | | TCTTGTGCTATTGGCAAAACCTATGGTA |
| | | | | | | | | | ACTCTTTAGGTATTCCTTCGAACAAGAT |
| | | | | | | | | | GCAAGAATAGACAAAAATGACAGCCCT |
| | | | | | | | | | TCTACGAGTGATTAGCCTGGTCGTGA |
| 2.68 | 3.90 | 0.86 | 12.44 | 4E+06 | STM3900 | | | | |
| 12.91 | 0.92 | 6.05 | 3.74 | 4E+06 | STM3908 | | | | |
| 13.98 | 1.29 | 6.05 | 3.81 | 4E+06 | IR STM3908- | ilvY | positive | − | GGCCGAGATCTTCTTCCAGCCGCTGAA |
| | | | | | STM3909 | | regulator | | TCTGCCGGGAGAGCGTGGAGGGGCTG |
| | | | | | | | for ilvC | | ACGTGCATCGCCCGCGCGCTGCGGCC |
| | | | | | | | (LysR | | AAAGTGGCGGCTTTCCGCCAGATGCAA |
| | | | | | | | family) | | GAAGGTTTTTAGATCGCGTAAATCCAC |
| | | | | | | | | | AGACAGACCTCCGGTTTTTGACGTTGC |
| | | | | | | | | | ATAAACCGCAACATAACGTTGTGAATAT |
| | | | | | | | | | ATCAATTTCCGCAATAAATTTCCTGTTG |
| | | | | | | | | | TAATGTGGGTTCATTCGCACAGATAGC |
| | | | | | | | | | AATCTGTAAACCGAACAATAAGCGCGA |
| | | | | | | | | | CACACAACATCACGGAGTACACCATCA |
| | | | | | | | | | TGGC |
| 18.44 | 2.07 | 7.27 | 1.04 | 4E+06 | STM3909 | | | | |
| 4.88 | 2.98 | 3.83 | 2.83 | 4E+06 | STM3945 | | | | |
| 2.89 | 3.25 | 2.76 | 2.32 | 4E+06 | IR STM3945- | STM3945 | pseudogene | − | AAAGATTGTTCTCCTCTTCTGGCTGGA |
| | | | | | STM3946 | | | | GATAAACCACGCCGCTGCCTTGCCGCT |
| | | | | | | | | | GATAAACATTGTGCGGAGATTCACTCA |
| | | | | | | | | | GCCGGCATCCCCAGGCGGGAGGCAGC |
| | | | | | | | | | AGAAGTGAAAGCGAAAAAAGGCAAAAC |
| | | | | | | | | | AAATTACGATATTGCATAAGGTCATCCG |
| | | | | | | | | | GACGTGGTACGTAAACCTAAAGTGATG |
| | | | | | | | | | AGCAAAGCATGTTTCCTGATGTAAATG |
| | | | | | | | | | CGCAATAATCATGGCAACGCGCCGCTT |
| | | | | | | | | | TTCAGATTTTATAAAGAGCCCCTAAACG |
| | | | | | | | | | CTTGCTTTTACGCCTTCTCCTGCGATGA |
| | | | | | | | | | TA |
| 2.55 | 9.80 | 1.68 | 16.67 | 4E+06 | STM3969 | | | | |
| 3.08 | 9.01 | 1.87 | 14.75 | 4E+06 | IR STM3969- | yigN | putative | + | GGAACAGGCCGTTACGCAAGATGAAGA |
| | | | | | STM3970 | | inner | | ATATCGTTTACGATCGATCCCTGAAGG |
| | | | | | | | membrane | | GCGGCAGGATGAACATTATCCCAATGA |
| | | | | | | | protein | | TGAACGGGTGAAGCAGCAGTTAAGTTA |
| | | | | | | | | | ACCCATACGGAGTAGTTTAGTCCTGGC |
| | | | | | | | | | GCAGAGTAGGGCAAATTGGCCCAATCT |
| | | | | | | | | | GTTACACTTCTTGAACATTTTTATCGAT |
| | | | | | | | | | AAGCAGGCACTGAGATGGTGGAAGATT |
| | | | | | | | | | CACAAGAAACGACGCACTTTGGCTTTC |
| | | | | | | | | | AGACCGTCGCTAAAGAGCAGAAAGCTG |
| | | | | | | | | | ACATGGTGGCCCACGTTTTTCATTCTGT |
| | | | | | | | | | GG |
| 5.95 | 2.88 | 1.38 | 5.00 | 4E+06 | STM3970 | | | | |
| 12.99 | 3.71 | 3.09 | 8.30 | 4E+06 | STM4031 | | | | |
| 12.92 | 3.54 | 3.24 | 7.75 | 4E+06 | IR STM4031- | STM4031 | putative | − | GTGAAGGAATATACCGCTTCATCTCTTC |
| | | | | | STM4032 | | cytoplasmic | | AGGCTGAGTGAATGTTTTTTTCTCCAGA |
| | | | | | | | protein | | ACATTCAGCAACTCAGTGAGAGCAAGC |
| | | | | | | | | | TCATGGTTTGGATACATGAGCATCGCT |
| | | | | | | | | | TCATTGAACGGTTTTCGGCTGATAACAT |
| | | | | | | | | | GCACAATGTAGTTCCATTACAAAGTTTT |
| | | | | | | | | | CAACCTGAAAACAATTTAGCGCAACGT |
| | | | | | | | | | TATCCAGTTTTCAAGTTGAAAACAAAAT |
| | | | | | | | | | TGAATTTTAGGTCATTTTGCCTGTTGAT |
| | | | | | | | | | GGACTTACAACACGCCAGGCCACATCT |
| | | | | | | | | | CGCATGGCGCTTCGTGCCGCCTGGC |
| 12.92 | 3.43 | 2.98 | 6.57 | 4E+06 | STM4032 | | | | |
| 7.75 | 2.89 | 1.60 | 12.31 | 4E+06 | STM4039 | | | | |
| 9.07 | 2.94 | 1.78 | 7.61 | 4E+06 | IR STM4039- | STM4039 | putative | − | TACAGGTTGTTCGTCCGCTTTTTTTTCA |
| | | | | | STM4040 | | inner | | TCACAAGCGCTTAGCCCGGCAGTCATC |
| | | | | | | | membrane | | AGCATAGCGATAATAATTGATGATAACA |
| | | | | | | | lipoprotein | | AATCCTTTTTCATTAGAATAACCTATAAA |
| | | | | | | | | | TAATATCATTGAAATTTACAGATTCATTT |
| | | | | | | | | | TAATGAAAAAAAACAGGTATGTGATTTA |
| | | | | | | | | | TTCAACACAAAAAATACTTAATGCATAT |
| | | | | | | | | | TTCATTATAATTAACATTATCAATATCAA |
| | | | | | | | | | TGTGTTCGTTAAAATAAGAGAACCCCAA |
| | | | | | | | | | CGTAAATATACAAAAGGCAATTAAATGA |
| | | | | | | | | | AAAGGAATTTATTATCCTC |
| 7.72 | 4.08 | 5.59 | 16.26 | 4E+06 | STM4073 | | | | |
| 8.23 | 5.98 | 5.62 | 12.28 | 4E+06 | IR STM4073- | ydeW | putative | − | TCAATCCATCGTGATAGTAGAACCAGG |
| | | | | | STM4074 | | transcriptional | | CAATACGCGCCACCTGCTCTTCTTCGC |
| | | | | | | | repressor | | ACATTCCATAATCAGATACCAACGTATT |
| | | | | | | | | | ATCGCTCATTGTCATAACCTGGCTTTAC |
| | | | | | | | | | TTTGAACATTTCTAAATCATTAACACAAT |
| | | | | | | | | | TGTTCAGTTATCACTCCGAAATAACCGT |
| | | | | | | | | | GATTAACGCCACAAAAACGCGCCAAAT |
| | | | | | | | | | CTGAACATTTATCATCTAAAAATTCATTT |
| | | | | | | | | | ATTCAGAAAACGTGATCTGGATGAGAG |
| | | | | | | | | | TTTTTTGACCAAATAACTACTACCGTTT |
| | | | | | | | | | TGAACAATTTCTTTTTCAAAAAA |
| 4.46 | 2.37 | 3.80 | 7.78 | 4E+06 | STM4074 | | | | |
| 3.25 | 3.43 | 3.30 | 3.62 | 4E+06 | IR STM4094- | cytR | transcriptional | − | CGCCTTCAACGCAACATCCTTCATCGT |
| | | | | | STM4095 | | repressor | | AGCGGCAGTAACCTGCTTGTTCGATTT |
| | | | | | | | (GalR/LacI | | CACTCTTTCTCCTCGCCTGGGAACTGC |
| | | | | | | | family) | | TGGCGCAGATCTATCCCTGGTAACACT |
| | | | | | | | | | CATCGAAAACATTTTTATCAGATAGTGC |
| | | | | | | | | | GTGGAAGCGGTTACAGAATTTTCATAA |
| | | | | | | | | | AAAGTGTGATGGATCTTTAATTTTACGA |
| | | | | | | | | | TCCGCCTCGCATCGTGAGGACTATCCT |
| | | | | | | | | | TCAATCGGATCGACGTCCAGAACCCAT |
| | | | | | | | | | TTAACTTTCCGCGCTTCCGGGAGCGTA |
| | | | | | | | | | TTGATCAACGCCAGCGTGCCGCTGATG |
| | | | | | | | | | AT |
| 5.79 | 3.45 | 4.28 | 5.46 | 4E+06 | STM4095 | | | | |
| 11.08 | 5.52 | 4.05 | 11.01 | 4E+06 | IR STM4111- | ptsA | General | − | TGCCTTTGCGATCGGTGCGCAGGTTGT |
| | | | | | STM4112 | | PTS family, | | GCCACTCAATTTGCGACGTGAAGGTAT |
| | | | | | | | enzyme I | | TACACAGCGTTTCTACGTGGCTTGCCG |
| | | | | | | | | | GGCGCGCATGTACGCCATTCGGCAGTT |
| | | | | | | | | | CACAGGTAAATTCCACAATCAGGGGCA |
| | | | | | | | | | TTGCCTCTCTCCCATAACGATTCTCTCG |
| | | | | | | | | | CTACAGCATAAAAGGAGGTAGCCGGAA |
| | | | | | | | | | TACGCCATGTGACAAATCTGTCAAAAG |
| | | | | | | | | | CTGGATAAATGTAATGTAGCGCAAAAA |
| | | | | | | | | | GTGCGAGTTGTCTCACAACTTAGCGTG |
| | | | | | | | | | GTAGCGCGGGTTTTACCTTTTTCAGAA |
| | | | | | | | | | GTT |
| 8.02 | 5.66 | 4.83 | 11.55 | 4E+06 | IR STM4146- | tufB | protein | + | TTGGCGCGGGCGTTGTTGCTAAAGTTC |
| | | | | | STM4147 | | chain | | TCGGCTAATCGCTGATAACATTTGACG |
| | | | | | | | elongation | | CAATGCGCAATAAAAGGGCATCATTTG |
| | | | | | | | factor EF- | | ATGCCCTTTTTGCACGCTTTCACACCA |
| | | | | | | | Tu | | GAACCTGGCTCATCAGTGATTTTATTTG |
| | | | | | | | (duplicate | | TCATAATCATTGCTGAGACAGGCTCTG |
| | | | | | | | of tufA) | | TAGAGGGCGTATAATCCGAAAGGCGAA |
| | | | | | | | | | TAAGCGTTTCGATTTGGATTGCCTCGC |
| | | | | | | | | | GATTGCGGGGTGAAAATGTTTGTAGAA |
| | | | | | | | | | TACTTCTGACAGGTTGGTTTATGAGTG |
| | | | | | | | | | CGAATACCGAAGCTCAAGGGAGCGGG |
| | | | | | | | | | CGCG |
| 7.78 | 8.04 | 6.00 | 15.15 | 4E+06 | STM4147 | | | | |
| 2.81 | 1.53 | 2.30 | 2.75 | 5E+06 | STM4263 | | | | |
| 4.46 | 4.38 | 4.91 | 4.25 | 5E+06 | IR STM4263- | yjcB | putative | − | TGTATTTTTTGTGCGTTTTATAACCGTA |
| | | | | | STM4264 | | inner | | TTTTTTGTGTGACTTCTACGCGTCCGTA |
| | | | | | | | membrane | | GAGAAACTGCCGGAAAGCAAAGATGTA |
| | | | | | | | protein | | TTATTACTACTCTTTTATTTTTTTTCGTG |
| | | | | | | | | | AAATTCAGACCTGATAAAAATATCAAGT |
| | | | | | | | | | TATTTATCAAAAGAAAGGAGTAAAGATG |
| | | | | | | | | | TATACCCCATCGTTTACTTGAGTATAAA |
| | | | | | | | | | TCTGATATTATCAAAAATATTTAGTGTC |
| | | | | | | | | | CTGCCTGGTATGCGAAAGAGATTGCGC |
| | | | | | | | | | GTAGTTATTAATGGTAAATGTTGATCGG |
| | | | | | | | | | TAAAAGTCTGTTGCTAATATTG |
| 2.64 | 9.15 | 5.09 | 10.54 | 5E+06 | STM4326 | | | | |
| 2.72 | 9.21 | 5.11 | 11.48 | 5E+06 | IR STM4326- | aspA | aspartate | − | GCCACGCACAAATTCAGGGATGTCGCT |
| | | | | | STM4327 | | ammonia- | | GATTTTGTTATTGCTAATGTAGAAGTTT |
| | | | | | | | lyase | | TCAATCGCTCTCAGAGTGTGAACACCA |
| | | | | | | | (aspartase) | | TAGTAGGCTTCAGCTGGAACTTCCCTG |
| | | | | | | | | | GTACCCAACAGATCTTCTTCGATACGA |
| | | | | | | | | | ATGTTGTTTGACATGTGAACCTTCTTTT |
| | | | | | | | | | TCAAGCTGCCAATGATTTTTACTTTAAA |
| | | | | | | | | | ACACACAGGATATATGTGATTTCGAATG |
| | | | | | | | | | TTTTCTGACCGACGATTATCCCCTCCAT |
| | | | | | | | | | CGGCCTGATAAACGAGATCATATGCTG |
| | | | | | | | | | GTTCAGAATTCCTACCGTAATCTGGA |
| 10.03 | 5.35 | 5.76 | 6.89 | 5E+06 | STM4382 | | | | |
| 10.43 | 4.51 | 5.76 | 6.05 | 5E+06 | IR STM4382- | yjfR | putative | − | GTACAGCCCAGCCACCACATAGCGAAC |
| | | | | | STM4383 | | Zn- | | GTACCCGGCGCGACCTGCTCTTGTTCA |
| | | | | | | | dependent | | ATCTCTTCGTTCAGCCAGCTTCCCCAC |
| | | | | | | | hydrolases | | TCCGGAAACGTGCTCAGAATCCATGAT |
| | | | | | | | of the beta- | | TCACGCGTGATGCTTTGTACTTTACTCA |
| | | | | | | | lactamase | | TCGCATTTACCTTCATGTTTGTTCAAAA |
| | | | | | | | fold | | TGGTTCAAAACGTGATTTGTTTTGATTA |
| | | | | | | | | | ATCCTGACACTATTTTCTCAAGAAGGCA |
| | | | | | | | | | ATGGGCTATTTTTTGACTTTTTGGAAGG |
| | | | | | | | | | AGAGAACGCAGTCAGGAGAAGATTTAA |
| | | | | | | | | | TCTTGTCTGGCGTCATGTGAATGTTT |
| 2.57 | 3.96 | 6.24 | 5.78 | 5E+06 | STM4383 | | | | |
| 6.23 | 5.41 | 2.09 | 10.97 | 5E+06 | IR STM4396- | ytfB | putative | − | TTGGTTTTAATTCAAAGCGCCCGGGCA |
| | | | | | STM4397 | | cell | | TGGTTTACCTCCTGCTCCGCATCTCGT |
| | | | | | | | envelope | | TCCTTAATCATAGAGTATAGATGGCTAA |
| | | | | | | | opacity- | | CGCTATGATACTGGTAGTGCTATCCGC |
| | | | | | | | associated | | TTTCGTGACATCAATACGGATAATCTAT |
| | | | | | | | protein A | | TGTTTCTTTTTCCCTGCGATTTGTCATC |
| | | | | | | | | | CTCCCTGAGACAAAGTTTTACCAGAAG |
| | | | | | | | | | AAGCGTGGCTGTTATGCTGCCCGCTAC |
| | | | | | | | | | TTTTTTGATATCCGATGAAGGAAAAATA |
| | | | | | | | | | ATGGCCACCCCGACTTTTGACACTATT |
| | | | | | | | | | GAAGCGCAAGCGAGCTACGGCATTGGT |
| 6.48 | 5.41 | 2.09 | 11.98 | 5E+06 | STM4397 | | | | |
| 5.26 | 4.17 | 1.76 | 5.57 | 5E+06 | STM4407 | | | | |
| 8.43 | 4.17 | 2.35 | 10.86 | 5E+06 | IR STM4407- | ytfL | putative | − | TAATAACTTAAGTTTAATCTTACGTGAT |
| | | | | | STM4408 | | hemolysin- | | GCGGCAAGCGAGATCTCGGAGATGGA |
| | | | | | | | related | | GAAGAACGCACTTACAGCGATCAGGCA |
| | | | | | | | protein | | GAATATAATGAATATACTGTTTAACATA |
| | | | | | | | | | TCTTATCCGGCGAAACGCCAGATCCTC |
| | | | | | | | | | GGAAGGGAAGTTTATAAATCCGTGTGG |
| | | | | | | | | | TAACGTTTAATGAAAACCGGCTCGTAG |
| | | | | | | | | | CAGTGAGCCGATAAGTTCAGGGCTAGT |
| | | | | | | | | | ATAGCGTAAGCTACTGTAAAGTCGCCA |
| | | | | | | | | | GAGGGTTCATTTTCAACTCCGACAAGT |
| | | | | | | | | | TCCCCCTACGCCAGCGTCGTCACGCGT |
| | | | | | | | | | CAG |
| 7.16 | 3.68 | 2.35 | 16.47 | 5E+06 | STM4408 | | | | |
| 16.03 | 2.44 | 1.33 | 7.29 | 5E+06 | STM4408 | | | | |
| 23.39 | 2.09 | 0.54 | 6.79 | 5E+06 | IR STM4408- | msrA | peptide | − | CCCGAAAGCGTTAATTGGCGTTAAGGT |
| | | | | | STM4409 | | methionine | | TGTAACGAGACGCATCTTTGCACACAA |
| | | | | | | | sulfoxide | | TAACAACATTAATGTATCTGGATTTAAC |
| | | | | | | | reductase | | CATAAGAAATATTTGGGCAGTCGTCTG |
| | | | | | | | | | CTTTTCAATCGAAATTGTTGATTTTATGT |
| | | | | | | | | | TAAGCCGCGGAGCGGTAGTGTGATTTT |
| | | | | | | | | | TTCCAGGGGTGGGAATAGGGGATATTC |
| | | | | | | | | | AGGAGAAAATGTGCCACATATCCGTCA |
| | | | | | | | | | GTTATGTTGGGTTAGCTTACTGTGCCT |
| | | | | | | | | | GAGCAGTTCTGCGGTAGCCGCAAATGT |
| | | | | | | | | | TCGTCTGAAAGTCGAAGGGCTATCCGGA |
| 23.39 | 2.11 | 0.59 | 6.79 | 5E+06 | STM4409 | | | | |
| 9.38 | 2.77 | 1.77 | 6.46 | 5E+06 | IR STM4416- | mpl | UDP-N- | + | ACGTCATCTTCTGCCTTTCAACGTTTGC |
| | | | | | STM4417 | | acetylmuramate:L- | | GATGCCGCCTGGCTGCGGGCATCGTC |
| | | | | | | | alanyl- | | CAGTCATAACAATGCTGATCCTGTCGC |
| | | | | | | | gamma-D- | | ATTTATGCGGTCAGATTCAGATTGCTCA |
| | | | | | | | glutamyl- | | GAACCCAGCCCGCCAGCAAATTCTGTA |
| | | | | | | | meso- | | CTGAAGGTAACCACAGCGCAATTTGAA |
| | | | | | | | diaminopimelate | | TGTTGTTAACTGTATGTTCAGTTCATTT |
| | | | | | | | ligase | | GTGCTAATATGGTTATTTACGAAATTTT |
| | | | | | | | | | CGTTCTATTAGAGTATCATGCATGTCTA |
| | | | | | | | | | AACATCAAACTCAACTTTCCTTACTGCA |
| | | | | | | | | | GGATGATATCCGCAGTCGCTATGACA |
| 9.63 | 3.11 | 1.87 | 5.93 | 5E+06 | STM4417 | | | | |
| 3.07 | 3.12 | 0.52 | 4.64 | 5E+06 | STM4473 | | | | |
| 3.19 | 2.34 | 0.42 | 4.90 | 5E+06 | IR STM4473- | yjgM | putative | − | GGTAAGTCCGTATTCCGCTGAAACCTG |
| | | | | | STM4474 | | acetyltransferase | | ACGGATGACACGGGCAATAGCGGCATT |
| | | | | | | | | | GTCGGCGGTAGTGATTCGGCGCACCG |
| | | | | | | | | | TGAGCGTTGGCGAGGCGACATTATTCA |
| | | | | | | | | | TAATATGGCTCAATTTTTAAAATTTATTT |
| | | | | | | | | | ATAGATTACTTTAATACCACCGTCTTGA |
| | | | | | | | | | GTTACGCGCAAGGAGATCCTGAATCAG |
| | | | | | | | | | ACAAAATAAAAGGCGGAAAAATTAAACA |
| | | | | | | | | | AAAATAGTATCGTAGTCAAATCAGTAAC |
| | | | | | | | | | AGTTTACTGGTTTTTATTATTAATTCTAA |
| | | | | | | | | | TAGATTGTAATTCAGGGATATGATT |
| 4.42 | 2.41 | 5.25 | 6.54 | 5E+06 | IR STM4501- | STM4501 | putative | − | TGTTCCTGACGGGATAAATTCATACTGA |
| | | | | | STM4502 | | cytoplasmic | | AGAACCTGTTTAATCATCATAGGCTAAA |
| | | | | | | | protein | | CGTGCAAACACACTGCGGTGTCCGCAT |
| | | | | | | | | | TCGATTTCGGCGCATTGATAATCAGTC |
| | | | | | | | | | CGGCCTGAAAAGGTCGGGTAACTGATT |
| | | | | | | | | | ATCAGATGATGACATTCTCCAGCATCAA |
| | | | | | | | | | AGCCTCGGGTTGAGTTGAAAGGTATTT |
| | | | | | | | | | ACGTCGTGAATGATAACACCTGATTTCT |
| | | | | | | | | | GTAAGTGAATAACCGGGAGTGAAAAGT |
| | | | | | | | | | GTGATCTCAAAGGGAGGCTCATGACGT |
| | | | | | | | | | TTAGCGTATCAGATGAATAGCTCCCGC |

TABLE 3B
Regions that induce GFP expression in both tumor and spleen (cont'd, presented in the same order as Table 3A)

| | | 3′ gene |
|---|---|---|
| 3′ gene | Function | orientation |
| STM0649 | putative hydrolase N-terminus | + |
| hutU | pseudogene; frameshift relative to Pseudomonas putida urocanate hydratase (HUTU) (SW: P25080) | + |
| STM1056 | Gifsy-2 prophage; homologue of msgA | − |
| STM1265 | putative response regulators consisting of a CheY-like receiver domain and a HTH DNA-binding domain | + |
| ydgF | putative membrane transporter of cations and cationic drugs | + |
| pspD | phage shock protein | − |
| STM1698 | putative inner membrane protein | − |
| nhaB | NhaB family of transport protein, Na+/H+ antiporter, regulator of intracellular pH | + |
| STM1839 | putative periplasmic or exported protein | − |
| yegE | putative PAS/PAC domain; Diguanylate cyclase/phosphodiesterase domain 1, Diguanylate | + |
| | cyclase/phosphodiesterase domain 2, | |
| cdd | cytidine/deoxycytidine deaminase | + |
| yfgB | putative Fe—S-cluster redox enzyme | − |
| gshA | gamma-glutamate-cysteine ligase | − |
| deaD | cysteine sulfinate desulfinase | − |
| hopD | leader peptidase HopD | + |
| pckA | phosphoenolpyruvate carboxykinase | + |
| ftsX | putative integral membrane cell division protein | − |
| yhjS | putative cytoplasmic protein | + |
| STM3624A | putative protein | + |
| rpmH | 50S ribosomal subunit protein L34 | + |
| cyaA | adenylate cyclase | + |
| udp | uridine phosphorylase | + |
| yiiU | putative cytoplasmic protein | + |
| rsd | regulator of sigma D, has binding activity to the major sigma subunit of RNAP | − |
| ecnB | putative entericidin B precursor | + |
| ytfF | putative cationic amino acid transporter | − |
| ytfK | putative cytoplasmic protein | + |
| idnK | D-gluconate kinase, thermosensitive | + |
| STM4552 | putative inner membrane protein | + |
| deoC | 2-deoxyribose-5-phosphate aldolase | + |
| PSLT048 | alpha-helical coiled coil protein | + |
| djlA | DnaJ like chaperone protein | + |
| stfA | putative fimbrial subunit | + |
| frr | ribosome releasing factor | + |
| uppS | undecaprenyl pyrophosphate synthetase (di-trans,poly-cis-decaprenylcistransferase) | + |
| yaeQ | putative cytoplasmic protein | + |
| STM0307 | homology to Shigella VirG protein | − |
| STM0341 | putative inner membrane protein | + |
| STM0343 | putative Diguanylate cyclase/phosphodiesterase domain 1 | + |
| phoB | response regulator in two-component regulatory system with PhoR (or CreC), regulates pho regulon | + |
| | (OmpR family) | |
| cypD | peptidyl prolyl isomerase | + |
| ybaY | glycoprotein/polysaccharide metabolism | + |
| acrR | acrAB operon repressor (TetR/AcrR family) | + |
| aefA | putative small-conductance mechanosensitive channel | + |
| cysS | cysteine tRNA synthetase | + |
| fepE | ferric enterobactin (enterochelin) transporter | + |
| cobC | alpha ribazole-5′-P phosphatase in cobalamin synthesis | − |
| kdpE | response regulator in two-component regulatory system with KdpD, regulates kdp operon encoding a high- | − |
| | affinity K translocating ATPase (OmpR family) | |
| STM0763.s | transcriptional regulator | − |
| STM0835 | putative Mn-dependent transcriptional regulator. | + |
| STM0860 | putative inner membrane protein | − |
| yljA | putative cytoplasmic protein | + |
| STM0947 | putative integrase protein | − |
| lrp | regulator for lrp regulon and high-affinity branched-chain amino acid transport system; mediator of of | + |
| | leucine response (AsnC family) | |
| serS | serine tRNA synthetase; also charges selenocystein tRNA with serine | + |
| ycaO | putative cytoplasmic protein | − |
| STM1001 | putative leucine response regulator | − |
| STM1020 | Gifsy-2 prophage | + |
| sulA | suppressor of lon; inhibitor of cell division and FtsZ ring formation upon DNA damage/inhibition, HsIVU and | − |
| | Lon involved in its turnover | |
| copS | Copper resistance; histidine kinase | − |
| ycdF | pseudogene; in-frame stops following codons 5 and 21 | + |
| rluC | 23S rRNA pseudouridylate synthase | + |
| potB | ABC superfamily (membrane), spermidine/putrescine transporter | − |
| STM1263 | putative periplasmic protein | + |
| yeaR | putative cytoplasmic protein | + |
| celA | PTS family, sugar specific enzyme IIB for cellobiose, arbutin, and salicin | + |
| ydiM | putative MFS family transport protein | − |
| ydiJ | paral putative oxidase | + |
| pykF | pyruvate kinase I (formerly F), fructose stimulated | − |
| orf242 | putative regulatory proteins, merR family | − |
| ydhL | putative oxidoreductase | + |
| malY | pseudogene; in-frame stop following codon 16 | − |
| ydgC | putative inner membrane protein | + |
| yncC | putative regulatory protein, gntR family | − |
| ynaF | putative universal stress protein | + |
| adhE | iron-dependent alcohol dehydrogenase of the multifunctional alcohol dehydrogenase AdhE | + |
| hnr | Response regulator in protein turnover: mouse virulence | − |
| STM1786 | hydrogenase-1 small subunit | + |
| STM1795 | putative homologue of glutamic dehyrogenase | + |
| minC | cell division inhibitor; activated MinC inhibits FtsZ ring formation | + |
| yobG | putative inner membrane protein | − |
| STM1841 | putative outer membrane or exported | + |
| STM1856 | putative cytoplasmic protein | + |
| pagK | PhoPQ-activated gene | + |
| STM1934 | putative outer membrane lipoprotein | + |
| fliB | N-methylation of lysine residues in flagellin | − |
| STM1967 | putative 50S ribosomal protein | + |
| STM2148 | putative periplasmic protein | + |
| yehV | putative transcriptional repressor (MerR family) | + |
| yohJ | putative effector of murein hydrolase LrgA | + |
| yejL | putative cytoplasmic protein | + |
| STM2281 | putative transcriptional regulator, LysR family | + |
| yfbQ | putative aminotransferase (ortho), paral putative regulator | + |
| yfcX | paral putative dehydrogenase | − |
| nupC | NUP family, nucleoside transport | + |
| yffB | putative glutaredoxin family | + |
| ndk | nucleoside diphosphate kinase | − |
| hmpA | dihydropteridine reductase 2 and nitric oxide dioxygenase activity | + |
| gogB | Gifsy-1 prophage: leucine-rich repeat protein | + |
| STM2621 | Gifsy-1 prophage | − |
| nadB | quinolinate synthetase, B protein | + |
| yfiO | putative lipoprotein | + |
| ygaM | putative inner membrane protein | + |
| proV | ABC superfamily (atp_bind), glycine/betaine/proline transport protein | + |
| hilD | regulatory helix-turn-helix proteins, araC family | + |
| STM2904 | putative ABC-type transport system | + |
| STM2954.1n | hypothetical protein | − |
| kduD | 2-deoxy-D-gluconate 3-dehydrogenase | − |
| yohM | putative inner membrane protein | + |
| ygfE | putative cytoplasmic protein | + |
| rpiA | ribosephosphate isomerase, constitutive | − |
| STM3084 | putative regulatory protein, gntR family | − |
| STM3169 | putative dicarboxylate-binding periplasmic protein | + |
| yqiC | putative cytoplasmic protein | + |
| ygiM | putative SH3 domain protein | + |
| yqjI | putative transcriptional regulator | + |
| rnpB | regulatory RNA | + |
| yhbY | putative RNA-binding protein containing KH domain | + |
| STM3343 | putative cytoplasmic protein | − |
| STM3357 | putative regulatory protein, gntR family | − |
| accB | acetylCoA carboxylase, BCCP subunit, carrier of biotin | + |
| def | peptide deformylase | + |
| slyX | putative cytoplasmic protein | + |
| hofQ | putative transport protein, possibly in biosynthesis of type IV pilin | − |
| yrfF | putative inner membrane protein | + |
| feoA | ferrous iron transport protein A | + |
| gntT | GntP family, high-affinity gluconate permease in GNT I system | + |
| livF | ABC superfamily (atp_bind), branched-chain amino acid transporter, high-affinity | − |
| uspA | universal stress protein A | + |
| STM3631 | putative xanthine permease | − |
| mtlA | PTS family, mannitol-specific enzyme IIABC components | + |
| STM3794 | putative regulatory protein, deoR family | + |
| torD | cytoplasmic chaperone which interacts with TorA | − |
| STM3858 | putative phosphotransferase system fructose-specific component IIB | − |
| ilvL | ilvGEDA operon leader peptide | + |
| ilvC | ketol-acid reductoisomerase | + |
| yifL | putative outer membrane lipoprotein | + |
| ubiE | S-adenosylmethionine: 2-DMK methyltransferase and 2-octaprenyl-6-methoxy-1,4-benzoquinone | + |
| | methylase | |
| STM4032 | putative acetyl esterase | − |
| yiiG | putative cytoplasmic protein | + |
| ego | putative ABC-type sugar, aldose transport system, ATPase component | + |
| priA | primosomal protein N′ (=factor Y) directs replication fork assembly at D-loops | − |
| frwC | PTS system fructose-like IIC component | + |
| secE | preprotein translocase IISP family, membrane subunit | + |
| yjcC | putative diguanylate cyclase/phosphodiesterase | + |
| fxsA | suppresses F exclusion of bacteriophage T7 | + |
| sgaT | putative PTS enzyme IIsga subunit | + |
| fklB | FKBP-type 22 KD peptidyl-prolyl cis-trans isomerase (rotamase) | + |
| msrA | peptide methionine sulfoxide reductase | − |
| ytfM | putative outer membrane protein | + |
| STM4417 | putative transcriptional regulator | + |
| yjgN | putative inner membrane protein | + |
| STM4502 | putative cytoplasmic protein | + |

TABLE 4
Intergenic regions that induce higher GFP expression in spleen than in tumor

| | | | |
|---|---|---|---|
| | Tumor | Tumor | |
| Spleen | (+) | (+)(−)(+) | |
| lib1 | lib2 | lib3 | Genome |

| | | |
|---|---|---|
| Median of | Tumor | position |
| experiment versus | (+)(−)(+) | of |
| input library | lib4 | peak |

| | | | | |
|---|---|---|---|---|
| lib-1 | lib-2 | lib-3 | lib-4 | signal |

| | | | | | | | | |
|---|---|---|---|---|---|---|---|---|
| | moving | moving | moving | moving | | | | |
| Clone | median | median | median | median | | Gene | Gene | |
| ID | of 10 | of 10 | of 10 | of 10 | Gene | symbol | orient. | Sequence |

| | | | | | | | | |
|---|---|---|---|---|---|---|---|---|
| 16.24 | 0.84 | 0.41 | 0.37 | 7389 | STM0006 | yaaJ | − | |
| 22.42 | 1.98 | 0.38 | 0.33 | 7513 | IR STM0006- | | | GTATTTCGTTAATAAAACTGAAAAAC |
| | | | | | STM0007 | | | TCAGGCATTAACGTCCCTCTTGTTG |
| | | | | | | | | ATGCCGGCACGCTTTGATAATCCTG |
| | | | | | | | | TATAAGCGTGACCCATGATGTAGAT |
| | | | | | | | | GACCTTGTCAGACTAATATTAACGG |
| | | | | | | | | CAGTTTACCATAAATACGGTGGTAT |
| | | | | | | | | CCTTTAATTGCGCATCAACCGTCGG |
| | | | | | | | | CAGATACGCAAACAGTGCACAAGG |
| | | | | | | | | GCAGCCAGGTGCATGTAGGCGGTT |
| | | | | | | | | GCGCTGTGAGTGCGTCGTGTTATCA |
| | | | | | | | | TCAGGGTAGACCGGTTACATCCCCT |
| | | | | | | | | AACAAGCTGTTTAAAGAGAAACTCT |
| | | | | | | | | AT |
| 21.01 | 1.73 | 0.38 | 0.30 | 7662 | STM0007 | talB | + | |
| 1.58 | 0.92 | 1.20 | 0.38 | 93836 | STM0080 | | + | |
| 20.94 | 0.46 | 0.93 | 0.29 | 94051 | IR STM0080- | | | TGCGAATAAACGGATGCCTGAACAG |
| | | | | | STM0081 | | | GCAGGGACGCCGGAAAACGTCGAA |
| | | | | | | | | ATACGTTAGACCATTCGCCCGTGTT |
| | | | | | | | | CCCGCTTTCCCCACCGCGCTGTCC |
| | | | | | | | | GCTTACATGAGGTTACACTCATCGA |
| | | | | | | | | CATTTCTCTGAACAGCGGCTCAACA |
| | | | | | | | | TTTCCCGGAAAAAAACATATCGCAG |
| | | | | | | | | GGCATTTATCCTTATGATTAGGTATA |
| | | | | | | | | AATGATGAGGTATAAGGAACAGGAG |
| | | | | | | | | TCTGTAATGAAACCAATACCTTTTTA |
| | | | | | | | | TTTGCTCGCGCTATTTTCTGCCGCC |
| | | | | | | | | TCCGGGGCTACGGAGATAAACGTC |
| | | | | | | | | TG |
| 25.94 | 0.56 | 1.06 | 0.31 | 94098 | STM0081 | | + | |
| 17.77 | 1.63 | 2.35 | 0.31 | 442273 | STM0390 | aroM | + | |
| 14.65 | 0.81 | 0.65 | 0.28 | 442548 | IR STM0390- | | | TCAAGGCGCGGACGTCATTATGCT |
| | | | | | STM0391 | | | GGATTGTCTGGGTTTTCATCAGCGT |
| | | | | | | | | CATCGGGATATTTTACAGCAGGCGC |
| | | | | | | | | TGGATGTGCCGGTTTTACTCTCTAA |
| | | | | | | | | CGTTTTGATTGCGCGGTTAGCTTCA |
| | | | | | | | | GAACTGCTTGTCTAATTTTACGTGA |
| | | | | | | | | CAGGCCGAACGTCAGGACTCTATAT |
| | | | | | | | | TGGGTGTTAATTTAATAATGAGACG |
| | | | | | | | | GGGCCTGATTATGCTACAAAGCAAT |
| | | | | | | | | GAATACTTTTCCGGGAAAGTTAAGT |
| | | | | | | | | CTATTGGATTTACCAGCAGTAGCAC |
| | | | | | | | | CGGCCGGGCCAGCGTTGGTGTGAT |
| | | | | | | | | GGC |
| 8.00 | 0.73 | 0.68 | 0.29 | 442570 | STM0391 | yaiE | + | |
| 9.82 | 1.66 | 0.42 | 0.52 | 667851 | STM0605 | ybdN | − | |
| 9.82 | 1.76 | 0.43 | 0.61 | 667878 | IR STM0605- | | | CAACGTTGCCGTCAGGTGCAACATA |
| | | | | | STM0606 | | | AGTCCTGAATCTTTACCACCAGAAA |
| | | | | | | | | ATGAGACGCAGACCCGGGGTAAGG |
| | | | | | | | | TTTCCAGGGTCCACATTATACGCTC |
| | | | | | | | | TTGAGCCGCTTCCAGAACATTTTGC |
| | | | | | | | | TCGAGCGGAACTTTATAAACCGACA |
| | | | | | | | | TCTCTGGATAGTCTCCGATGTGTTA |
| | | | | | | | | ACTACAGTATATTCGAAATAATTAAC |
| | | | | | | | | ATAAAGGATAAGCAGATTAGATGAA |
| | | | | | | | | CTTGCAATGCTTTATTATATTTGTAA |
| | | | | | | | | AATAAATATATTCCATAAACATATAC |
| | | | | | | | | ATTAAATTTATATTAATATCCGTT |
| 4.72 | 0.66 | 0.90 | 0.70 | 668757 | STM0606 | ybdO | − | |
| 15.90 | 0.66 | 0.71 | 0.25 | 962476 | STM0892 | ybjP | − | |
| 10.80 | 0.44 | 0.63 | 0.31 | 962530 | IR STM0892- | | | TGAGCCACGCTGTCCGGGCCGCCT |
| | | | | | STM0893 | | | TCCACACACGCGCCGATACGCGGG |
| | | | | | | | | CCATTATCTTTGTAGGCGGGAGTGA |
| | | | | | | | | CGGTCGTACAGGCGCTAAGCAGAA |
| | | | | | | | | GCGCGCACGGGATGAGCAAAGAGA |
| | | | | | | | | GTTTAGAATAGCGCATGATGATTTC |
| | | | | | | | | CTTATAGGCGATCGAGCAAAAACCG |
| | | | | | | | | ATCTACGATAATCAATTATATCCTTT |
| | | | | | | | | CAGTGATTGCATAACCACTTAACAT |
| | | | | | | | | CTTGTTTTATCTAAATAAAATTAAGC |
| | | | | | | | | ATGTTATCTTTTTGGGGCACTCCTG |
| | | | | | | | | GGGCAGTAGATGCCAGTTGTTGATT |
| | | | | | | | | CAG |
| 6.64 | 0.41 | 0.75 | 0.58 | 962570 | STM0893 | | − | |
| 5.69 | 0.32 | 0.27 | 0.39 | 1E+06 | STM1044 | sodC | − | |
| 8.09 | 0.63 | 0.32 | 0.39 | 1E+06 | IR STM1044- | | | ATGTTTTCTCCTGTTCCGCTGGACA |
| | | | | | STM1045 | | | GGGCATCGTTCATCTTTACAGTCAG |
| | | | | | | | | GGTATTCTCTGCCATTGCTGAACAA |
| | | | | | | | | CTGATGAGCGCACCAGCTACCAGC |
| | | | | | | | | GACAATATTGTGTATTTCATTAGTTA |
| | | | | | | | | CCTCGTTTTTTGGTTGTATCGTAAAT |
| | | | | | | | | ACCATTAATAAAAGCAGGTATATGTT |
| | | | | | | | | TGCAAGATAAATAATAAAGGATCTC |
| | | | | | | | | TCATATATGCAGGATATACCACAGG |
| | | | | | | | | AAACCCTGAGCGAGACCACCAAAG |
| | | | | | | | | CGGAGCAGTCCGCGAAGGTGGATT |
| | | | | | | | | TGTGGGAATTTGATTTAACCGCGATT |
| 10.05 | 0.88 | 0.38 | 0.50 | 1E+06 | STM1045 | | + | |
| 12.79 | 0.74 | 1.01 | 0.23 | 1E+06 | STM1231 | phoP | − | |
| 12.76 | 0.74 | 0.45 | 0.23 | 1E+06 | IR STM1231- | | | AGGTGTTCATTAAGGTAGTAATCAG |
| | | | | | STM1232 | | | CTTCCCTGGCATCTTCTGCGGCATC |
| | | | | | | | | GACCTGGTGACCTGAATCCTGGAG |
| | | | | | | | | CTGAACCTTCAGGTGGTGGCGTAAT |
| | | | | | | | | AATGCATTATCCTCTACAACCAGTA |
| | | | | | | | | CGCGCATCATCTCTTCTCCCTTGTG |
| | | | | | | | | TTAACAATAAGAACAGTCTAGCGTT |
| | | | | | | | | GATTATGGTGCTTTGGGGATAAACA |
| | | | | | | | | GTTAATAAACCAGACAAATAGTCAC |
| | | | | | | | | CCTCTTTCTGAAGAAAAGAGGGTGA |
| | | | | | | | | GGCAGGCATTATTTAAGTTCGTCGA |
| | | | | | | | | CCAGAGTCACAGCGCGACCGATAT |
| | | | | | | | | AAT |
| 9.96 | 0.61 | 0.45 | 0.30 | 1E+06 | STM1232 | purB | − | |
| 1.16 | 2.63 | 6.81 | 5.31 | 1E+06 | STM1249 | | − | |
| 31.95 | 0.64 | 1.01 | 0.40 | 1E+06 | IR STM1249- | | | TCAGTGAAACTATTTCTTCAAATGAT |
| | | | | | STM1250 | | | GGTCTTTTTATTATCGATCAGATAAT |
| | | | | | | | | GGCATCAACAGGGGTTATTCAGGA |
| | | | | | | | | GTATATGTGAAAAAGTGGCTTATAG |
| | | | | | | | | GAGGGATATTGATCGCAAGTTTTCT |
| | | | | | | | | GACCGGTTGTCTGATGTGGCACAA |
| | | | | | | | | CATTGATAAATGGTTTAATAAAGATA |
| | | | | | | | | TCGAATTTTTCTACGTCGGAGACGA |
| | | | | | | | | TAGCTAAAATTCCAGTCAGTTGGCA |
| | | | | | | | | ACGGGTGTCATATCTTCAGGTATGG |
| | | | | | | | | CGCCCGGAGCCGCCGGGCGCAAAT |
| | | | | | | | | TGTAGGTGTATAAAAGTCATTTCATT |
| 12.37 | 0.82 | 0.82 | 0.48 | 1E+06 | STM1250 | | + | |
| 11.46 | 1.34 | 0.41 | 0.33 | 2E+06 | STM1583 | | − | |
| 10.52 | 1.60 | 0.34 | 0.44 | 2E+06 | IR STM1583- | | | TGCGGTAAGCACATACAAGATGCCT |
| | | | | | STM1584 | | | TTCATGATTTTTGTTGATAATTTATTT |
| | | | | | | | | TCATAATCTCCTGCAGCAACATGAG |
| | | | | | | | | GTAGCTTATTTCCTGATAAAGCTCT |
| | | | | | | | | GGCATAGGTAGAAACTGATGTATAT |
| | | | | | | | | GGCATATCCTACTCCTTCAAATTTTG |
| | | | | | | | | CTCAATAGCTTTATATGTCCTACTCC |
| | | | | | | | | TCTCTCATTATGACGATATGTCAATC |
| | | | | | | | | AACAAAATTGCTCAAAGGCATACAT |
| | | | | | | | | TTTCAGGAGAAAATGAGAATAACAG |
| | | | | | | | | GCGCAACGGCCTGATCTTATGCTG |
| | | | | | | | | CTTCAATATCGTCAGGTGGTTT |
| 2.44 | 0.56 | 0.92 | 0.41 | 2E+06 | STM1584 | ansP | + | |
| 34.34 | 1.01 | 0.56 | 0.26 | 2E+06 | STM1736 | yciA | + | |
| 38.32 | 1.01 | 0.57 | 0.29 | 2E+06 | IR STM1736- | | | ACGACGTCTATTAGCATAAATATTG |
| | | | | | STM1737 | | | AAGTCTGGGTGAAAAAAGTCGCGTC |
| | | | | | | | | AGAACCGATTGGGCAGCGCTACAA |
| | | | | | | | | GGCCACCGAGGCGCTGTTTATTTAT |
| | | | | | | | | GTTGCCGTCGATCCGGACGGTAAA |
| | | | | | | | | CCTCGCCCGCTCCCGGTTCAGGGT |
| | | | | | | | | TAAGTATACCCGCTTACGCCGCCAG |
| | | | | | | | | CAGGTGATGGTATATTCCTGGCTGG |
| | | | | | | | | CGGCGCCAGAGATTACTCAATCTGC |
| | | | | | | | | GCCGTACCGTTCAGACGGAAGATA |
| | | | | | | | | ATATTGACCACCAGCCCGGAACCC |
| | | | | | | | | GGCTTGCCTGCTTCATAGCGCCATT |
| | | | | | | | | TTCGCA |
| 39.25 | 0.95 | 0.69 | 0.30 | 2E+06 | STM1737 | tonB | − | |
| 1.31 | 1.19 | 2.93 | 0.37 | 2E+06 | STM1868.1N | | − | |

| | | | | | | |
|---|---|---|---|---|---|---|
| 10.59 | 1.46 | 0.38 | 0.48 | 2E+06 | IR | GTTCGCCGTCCATTTTTACCTCTGG |
| | | | | | STM1868.1N- | GGCTGTTTCTTAGCGCGCCCTCCC |
| | | | | | STM1868A | CCGGAAAAACAAAATATAATGAACA |
| | | | | | | AAAAACATACAAACCATCATCTTTTA |
| | | | | | | AAAATAAATTACATTAAAACAGAGAG |
| | | | | | | TTACAACATGATGATGATGCATGAA |
| | | | | | | AAATCAAAAATGCGCCAAATCCCGC |
| | | | | | | GCCGCTGCCGCCCCGTGGCAGGC |
| | | | | | | CGCCCCGCCGGGAGTACCTTTTTAA |
| | | | | | | AATGCGAACAATTATCAACAACTAC |
| | | | | | | CACTTAATGATTATTTATTTCATTTT |
| | | | | | | GCGATATTGATTATCATTTTCAATAA |

| | | | | | | | | |
|---|---|---|---|---|---|---|---|---|
| 8.17 | 1.52 | 0.22 | 0.31 | 2E+06 | STM1868A | | + | |
| 11.80 | 1.45 | 0.68 | 0.33 | 2E+06 | STM1876 | holE | + | |
| 14.81 | 1.25 | 0.83 | 0.34 | 2E+06 | IR | | | GCTACAATATGCCAGTTGTCGCGGA |
| | | | | | STM1876- | | | GGCGGTCGAACGTGAGCAGCCAGA |
| | | | | | STM1877 | | | GCATCTACGCGCCTGGTTTCGCGA |
| | | | | | | | | GCGGCTGATTGCCCATCGTCTGGC |
| | | | | | | | | TTCCGTATCACTATCCCGACTCCCT |
| | | | | | | | | TACGAACCCAAAGTTAAATAAAAATT |
| | | | | | | | | ATATAACGTTACACTTCCTTACATGC |
| | | | | | | | | AGACGACTACATTATAAGGCGATTC |
| | | | | | | | | TTAACCTATGCTTTTTAGAATGGCTG |
| | | | | | | | | TAGAGACTATGAAAAGGAAGTCATT |
| | | | | | | | | ATGTCCTCCTGGAAAATTGCTGCTG |
| | | | | | | | | CGCAGTATGCGCCCCTGAACGCCT |
| | | | | | | | | CG |
| 12.07 | 0.81 | 0.97 | 0.37 | 2E+06 | STM1877 | | + | |
| 14.41 | 0.62 | 0.43 | 0.33 | 2E+06 | STM2153 | yehE | − | |
| 19.07 | 0.61 | 0.39 | 0.37 | 2E+06 | IR | | | GGTTAATGTTGCGGTGTCGGAGGC |
| | | | | | STM2153- | | | AAAAACAGGTACGCTTATCCCATAA |
| | | | | | STM2154 | | | GCCGAAACTATAATTCCCATCAGCA |
| | | | | | | | | AATATTTTTTCATAGTGAGTAATTGT |
| | | | | | | | | TCCTCTGGTGAACGTCAAACAGTAT |
| | | | | | | | | GCAGGCCGTCCTGATGAGCAGTAT |
| | | | | | | | | GAACGTATCGATACCTTAAAACCAA |
| | | | | | | | | TTGAAAAAATAAATCAGTAGGATAG |
| | | | | | | | | GTATGATCAATTCAAATAATGTTTTT |
| | | | | | | | | GCCGATTATTTCAGATAAACACCTG |
| | | | | | | | | TCTGTTTAAGCAGGAATTAACAATG |
| | | | | | | | | CGGGGGCTATTATTTTATTAATACAT |
| 4.64 | 1.02 | 0.57 | 0.41 | 2E+06 | STM2154 | mrp | − | |
| 11.33 | 1.37 | 0.82 | 0.45 | 2E+06 | STM2169 | yohC | − | |
| 11.99 | 1.53 | 0.81 | 0.45 | 2E+06 | IR | | | ACGACGGGAATCGCCGCCATCAGC |
| | | | | | STM2169- | | | AAAACATGGTGCGTATAGTGATGCG |
| | | | | | STM2170 | | | AAACAGTTTCGTTTTCGCTTTTGATC |
| | | | | | | | | ACCTGCATTTCCCGATCGGGATGG |
| | | | | | | | | GAAAAAAGCCCCCATACATGGTTCA |
| | | | | | | | | TACTGCCCCCTTCTGCTGCCTCAGA |
| | | | | | | | | TGCCAGTATGTTCAAGTATAATTCA |
| | | | | | | | | GTTTCTGGTTATTTTATGAACAATGG |
| | | | | | | | | CAAAATAGTCTCCGGCAAAACGTCG |
| | | | | | | | | GCTTTGCCGCGCACGCCTCTTGCC |
| | | | | | | | | AGGGTGTATGCTTAATGCCGGAGG |
| | | | | | | | | TGGTTTACGCATGGATATCAACACG |
| | | | | | | | | CTT |
| 11.13 | 1.58 | 0.80 | 0.47 | 2E+06 | STM2170 | yohD | + | |
| 20.97 | 0.90 | 1.83 | 0.42 | 2E+06 | STM2349 | yfcG | + | |
| 17.50 | 0.66 | 1.54 | 0.33 | 2E+06 | IR | | | GATCTTGATACCTACCCGGCGGTGT |
| | | | | | STM2349- | | | ATAACTGGTTTGAACGCATTCGCAC |
| | | | | | STM2350 | | | GCGTCCTGCGACAGCGCGCGCACT |
| | | | | | | | | GTTACAAGCGCAACTGCACTGTAAC |
| | | | | | | | | AGTACGAAAGCGTAACGCGGTAGC |
| | | | | | | | | ATACATCATGTATGATGTAGAGGTG |
| | | | | | | | | TATACACGGAAAAAACCTGCGTCCG |
| | | | | | | | | GCACCCTTATTCGTATTAAAAACCT |
| | | | | | | | | GACATTAGGGAAGAGGAAATCCTCC |
| | | | | | | | | CTACTCTGGAGGTCATATGCAGATT |
| | | | | | | | | CTGATTACCGGCGGTACAGGCCTG |
| | | | | | | | | ATAGGGCGTCATCTCATTCCCCGGC |
| | | | | | | | | TGTT |
| 13.83 | 0.67 | 1.52 | 0.33 | 2E+06 | STM2350 | yfcH | + | |
| 14.01 | 1.14 | 1.19 | 0.43 | 2E+06 | STM2366 | accD | − | |
| 11.78 | 1.29 | 1.15 | 0.39 | 2E+06 | IR | | | CTCAAGATTACGTTCCAGCTCAGCG |
| | | | | | STM2366- | | | CGGTATAAAACCTGACCGCAGCTAT |
| | | | | | STM2367 | | | CACACTTGGTCCACACCCCTTCAGG |
| | | | | | | | | AATGCTAGCCTTGCGGGTGGGAGT |
| | | | | | | | | AATGTTGCTTTTAATTCGTTCAATCC |
| | | | | | | | | AGCTCATTGGTGACCTTTCTGCCTG |
| | | | | | | | | AACCTTAGTCAGCTTTATTATAAGG |
| | | | | | | | | GGCGCATAATGCCATTTTTGCCCCC |
| | | | | | | | | AACAGACCATGAATGTTGCACATTA |
| | | | | | | | | AAACATAACAGCCCGAAACTTTGGA |
| | | | | | | | | TAAAAAAGTGGTCGAACCGCTGAGT |
| | | | | | | | | TACTTTCTATTTTGCGGCACGCGACG |
| 3.49 | 0.92 | 0.89 | 0.35 | 2E+06 | STM2367 | dedA | − | |
| 1.89 | 0.55 | 0.31 | 0.26 | 3E+06 | STM3047 | ygfY | − | |
| 10.99 | 0.73 | 0.24 | 0.26 | 3E+06 | IR | | | ATTGTGAATATCCATGTTCTTCCTGC |
| | | | | | STM3047- | | | CTCGCGAAAATGAAGTACCGGGCT |
| | | | | | STM3048 | | | ATTGTAACGTGTTTTTGGCGTTGTTT |
| | | | | | | | | TACGGGAATCTCAGTAATCTGGAAC |
| | | | | | | | | GCGATCGCGAAATAAAAGGCTGGG |
| | | | | | | | | AATCAATATGTTCATCCATTTTGGAT |
| | | | | | | | | ACCGCCTCGCAAAACGATCAATCCG |
| | | | | | | | | CTCTCAATGGGCTATTTAAAGCACT |
| | | | | | | | | TGCAATGACCGATGGCTCTTTTACC |
| | | | | | | | | ATTAACCATTATTGTTGCAGCTAACC |
| | | | | | | | | AGGACATTATTTATGGCTTTTATCTC |
| | | | | | | | | CTTTCCACCACGTCATCCTTCAT |
| 12.16 | 1.18 | 0.31 | 0.30 | 3E+06 | STM3048 | ygfZ | + | |
| 9.40 | 0.58 | 0.91 | 0.42 | 3E+06 | STM3231 | yqjK | + | |
| 14.81 | 0.63 | 1.13 | 0.54 | 3E+06 | IR | | | GGTCGGTAGCAGCGTAATGGCCAT |
| | | | | | STM3231- | | | CTGGACCATCCGTCATCCTAATATG |
| | | | | | STM3232 | | | TTGGTACGCTGGGCGAAACGCGGC |
| | | | | | | | | CTGGGTATCTGGAGCGCCTGGCGC |
| | | | | | | | | CTGGTAAAAACTACCCTCCGTCAAC |
| | | | | | | | | AACAGCTCCGCGGTTAATATCTTTT |
| | | | | | | | | CTTTTATAGCATCGCGCCATCAGGT |
| | | | | | | | | TATCACCTGGTGGCGCGATACTTTT |
| | | | | | | | | ATGCATATCGTCTCTTTAGCAATCA |
| | | | | | | | | CTCAAATTTTTTGAAAAAATTTGGCA |
| | | | | | | | | ATTTTCCTTGCTAACAATTCCTGCAC |
| | | | | | | | | GCCACGTTTATGATTCTCTCCAGCG |
| | | | | | | | | AT |
| 11.41 | 1.09 | 1.30 | 0.41 | 3E+06 | STM3232 | yqjF | + | |
| 2.83 | 0.88 | 1.96 | 0.25 | 4E+06 | STM3805 | yidH | − | |
| 10.53 | 0.55 | 1.90 | 0.28 | 4E+06 | IR | | | GACGCCTGCCGCCAGAAATCCCAG |
| | | | | | STM3805- | | | CGAGGTGCGAATCCACGCCAGAAA |
| | | | | | STM3806 | | | GGTGCGCTCATTTGCCAGTGAGAA |
| | | | | | | | | GCGATAATCCGGCGCTTCTCCGAG |
| | | | | | | | | GCGGGAAATCTTCATGACGACTCCT |
| | | | | | | | | TTTACGTTCTTATGTATTCCCGTTCG |
| | | | | | | | | TTTTCAGAATACCACTCACGTTGTT |
| | | | | | | | | GCTGATATGCTTCACATTATCCCGC |
| | | | | | | | | AGCAAGGGAATCTTATTGCAAAATA |
| | | | | | | | | ACTGTAGTTCACTGGTGATGCGTTT |
| | | | | | | | | TGGCGCAACCGCGCTCATTGCCGC |
| | | | | | | | | TATTTTTCATTTCAGTTACGACCTTT |
| | | | | | | | | TTCA |
| 14.49 | 0.95 | 0.95 | 0.37 | 4E+06 | STM3806 | | + | |
| 3.74 | 1.05 | 0.59 | 0.26 | 5E+06 | STM4286 | lpxO | − | |

| | | | | | | |
|---|---|---|---|---|---|---|
| 9.12 | 1.26 | 0.50 | 0.36 | 5E+06 | IR STM4286- | CGGTGATGCCAAAGAGAAAAGTGTA |
| | | | | | STM4287.S | GTTCGTTGACAATAAATTTACATTTC |
| | | | | | | TACAACTTAAAAGGGCCATTTTTGC |
| | | | | | | TAAAGAAGCGAGTCAGCCCGTTTAA |
| | | | | | | CCTTTATCCAGGCTTGTCGACAGTA |
| | | | | | | GAATTGAGATGACTCCGCTACTTCA |
| | | | | | | CCCGGTGATGGCTGATTACGTTATG |
| | | | | | | CCTTATCTCCCGATGACGGCTGCCA |
| | | | | | | GATCACAATGCTTTCGTAAACCGAA |
| | | | | | | AATGACTTTGCTTGTAACCTTCGCG |
| | | | | | | AAGATAAAAACGGTGTGCATCGCG |
| | | | | | | GCGTTTAATATTTGTGGAAAGCTCCG |

| | | | | | | | | |
|---|---|---|---|---|---|---|---|---|
| 9.12 | 1.29 | 0.50 | 0.36 | 5E+06 | STM4287 + | | | |
| | | | | | STM4287.S | | | |
| 7.62 | 1.72 | 0.64 | 0.41 | 5E+06 | STM4290 | proP | + | |
| 7.69 | 1.57 | 0.62 | 0.41 | 5E+06 | IR | | | GCGTCGGACATCCAGGAAGCGAAG |
| | | | | | STM4290- | | | GAAATTCTGGGCGAGCATTACGATA |
| | | | | | STM4291 | | | ATATTGAGCAGAAAATCGACGACAT |
| | | | | | | | | CGATCAGGAAATTGCGGAGCTGCA |
| | | | | | | | | GGTCAAACGTTCGCGTCTGGTACA |
| | | | | | | | | GCAACATCCGCGTATCGATGAATAA |
| | | | | | | | | ATTTCGCGCTTAAGGTTCGCTTAAT |
| | | | | | | | | CTCTCGCGGGCATACTCTCCTCCAT |
| | | | | | | | | ACCTTTGGAGGAGAGCGTCATGAAA |
| | | | | | | | | AGCTATATTTATAAAAGTTTGACGAC |
| | | | | | | | | CCTGTGTAGTGTGCTGATTGTCAGC |
| | | | | | | | | AGTTTTATCTATGTGTGGGTCACGA |
| | | | | | | | | CGT |
| 1.41 | 0.75 | 1.79 | 0.35 | 5E+06 | STM4291 | basS | − | |
| 18.03 | 1.30 | 0.20 | 0.27 | 5E+06 | STM4328 | yjeH | − | |
| 17.61 | 1.11 | 0.22 | 0.30 | 5E+06 | IR | | | GATGTGGTTAACAAGATAACGCCCT |
| | | | | | STM4328- | | | GAACCAACCCAAGCTCTTTTTTTAG |
| | | | | | STM4329 | | | TTCATTCATCAGCTCATTATCCGGC |
| | | | | | | | | GGCATTGTAACGTCAGGTGACGAC |
| | | | | | | | | AGACATTTTTAAGCGTATCACACAC |
| | | | | | | | | GCCTTTTCTTATAGCAGGATGTTCT |
| | | | | | | | | AAACCTTGGGTAAACGTGAGATAAG |
| | | | | | | | | TAGCGTTTTTACCGCTTTTTTCGCTC |
| | | | | | | | | AGAAGAATTTTTTTTCATCTCCCCCC |
| | | | | | | | | TTGAAGGGGCAAAACCCCATCCCC |
| | | | | | | | | ATCTCTCTGGTCACCAGCCGGGAAA |
| | | | | | | | | CCGTTTACGGGCCGGCGTCACCCA |
| | | | | | | | | TA |
| 2.21 | 1.06 | 0.57 | 0.48 | 5E+06 | STM4329 | mopB | + | |
| 28.58 | 0.84 | 1.28 | 0.56 | 5E+06 | STM4362 | hflX | + | |
| 35.05 | 1.86 | 1.16 | 0.37 | 5E+06 | IR | | | AGCGTCAGTCTGCAGGTACGAATG |
| | | | | | STM4362- | | | CCGATTGTCGACTGGCGTCGCCTC |
| | | | | | STM4363 | | | TGTAAACAAGAACCGGCGTTGATCG |
| | | | | | | | | AATACGTGATCTAGACGCGAAGTCA |
| | | | | | | | | TTCAGGTCGTATTGAGGCGGTAGCT |
| | | | | | | | | GGAGAGAATCTCAGGAGCTCACAA |
| | | | | | | | | CGAAGTGACCTGGGGTAAAAAAGC |
| | | | | | | | | CGCCACTCAAGACGCAGCCTGAAA |
| | | | | | | | | GATGATGTCTGTAACGGCGGTTCGT |
| | | | | | | | | CTGAAGCATGGAGTAATTTCGCCTT |
| | | | | | | | | ATCCTCTGAGGTCGAAAGACAACG |
| | | | | | | | | GGGATCACCGCATAACAAATATGGA |
| | | | | | | | | GCACAAA |
| 33.31 | 0.91 | 1.01 | 0.29 | 5E+06 | STM4363 | hflK | + | |
| 9.82 | 0.90 | 1.26 | 0.48 | 3113 | IR PSLT006- | | | AAACTGCCGCCGGAGCCGCGTGAA |
| | | | | | PSLT007 | | | AATATTGTTTATCAGTGCTGGGAAC |
| | | | | | | | | GTTTTTGCCAGGCATTGGGGAAAAC |
| | | | | | | | | CATCCCGGTGGCGATGACGCTGGA |
| | | | | | | | | AAAAAATATGCCGATTGGTTCCGGG |
| | | | | | | | | TTAGGGTCCAGCGCCTGTTCCGTC |
| | | | | | | | | GTCGCCGCGCTGGTCGCGATGAAT |
| | | | | | | | | GAGCACTGCGGCAAACCGTTAAAC |
| | | | | | | | | GACACGCGTCTGTTGGCGCTGATG |
| | | | | | | | | GGCGAGCTGGAAGGCCGTATCTCC |
| | | | | | | | | GGCAGCATCCATTACGATAACGTCG |
| | | | | | | | | CGCCGTGCTTTCTTGGCGGTATGCA |
| | | | | | | | | GTTGATGA |
| 2.88 | 0.48 | 0.74 | 0.34 | 3721 | PSLT007 | | + | |
| 7.69 | 0.92 | 1.67 | 0.45 | 17888 | IR PSLT024- | | | TCATTTTTATGATTTTTATATCATCTA |
| | | | | | PSLT025 | | | AAAAGATGATGTTTTGTGATTAGCTA |
| | | | | | | | | TTTTTTATGCCTGTAACGATTATGGA |
| | | | | | | | | CCCCGCAGAACGAGCTGCGACAAT |
| | | | | | | | | TTTGAAACGTAAAAGGAAATTTGAA |
| | | | | | | | | AATGGCTACAAGCAAACTGATTCAA |
| | | | | | | | | GGCGATACAATTACTGAAACTACTC |
| | | | | | | | | ATGCAGCGAATGGTTTTGACCCTGC |
| | | | | | | | | AACAAGCGATGATAAAATAAGCTAT |
| | | | | | | | | ACTTCCGCTCGTGTTGCGAAACCG |
| | | | | | | | | GTATACAATAAATATAAAAATTCCAC |
| | | | | | | | | GACTAAACCGAAGGTATTCGGTT |
| 5.19 | 0.66 | 1.53 | 0.40 | 18097 | PSLT025 | | − | |
| 3.20 | 1.01 | 0.82 | 0.38 | 18666 | IR PSLT025- | | | AACTGTTCAAACAGTTCCCGATGTT |
| | | | | | PSLT026 | | | CAGCGAAGTGGATATTGACTGGGA |
| | | | | | | | | ATACCCGAACAATGAAGGGGCGGG |
| | | | | | | | | CAACCCGTTTGGTCCGGAAGATGG |
| | | | | | | | | CGCTAACTACGCGCTGCTGATTGCC |
| | | | | | | | | GAACTGCGTAAACAGCTGGATTCCG |
| | | | | | | | | CGGGTCTGAGCAATGTGAAGATCTC |
| | | | | | | | | TATTGCCGCTTCTGCTGTCACTACT |
| | | | | | | | | ATTTTTGACTATGCGAAAGTAAAAG |
| | | | | | | | | ATCTGATGGCTGCCGGCCTGTATG |
| | | | | | | | | GCATCAACCTGATGACCTATGACTT |
| | | | | | | | | TTTCGGTACGCCGTGGGCGGAAAC |
| | | | | | | | | GCTGGG |
| 3.84 | 1.29 | 0.49 | 0.36 | 30863 | PSLT040 | spvA | − | |
| 12.30 | 0.93 | 1.84 | 0.37 | 31227 | IR PSLT040- | | | CGTGGCTCCCTTTGCAACGCGTCAA |
| | | | | | PSLT041 | | | ACGGACTGGTGCCGGCACACGGTT |
| | | | | | | | | CGCTGCACTGTGCGCTGGCAAAGT |
| | | | | | | | | ATTAATGACTATGGGCGGGTAATGC |
| | | | | | | | | CAGCGCAAACCGTGGATCTGACGC |
| | | | | | | | | GTATTCATTAACCTATTTTTCAGGCG |
| | | | | | | | | TCTCCCGATAGCGGGAGGCTTTCC |
| | | | | | | | | GAACTTATCGAACGAGACTTTTATTA |
| | | | | | | | | TGTATTATCACGCGTTAAAACTTTCC |
| | | | | | | | | CGACTGGCGATGTTGACGTTGGCA |
| | | | | | | | | GGCGTTGCCGTATCCGCCTCGGCA |
| | | | | | | | | ATCGCCGCCGATTCTGCCCCGACG |
| | | | | | | | | TCGCA |
| 7.27 | 1.02 | 3.20 | 0.51 | 31383 | PSLT041 | spvR | − | |
| 7.16 | 0.55 | 1.08 | 0.74 | 32347 | IR PSLT041- | | | TCCTTTATCGTTCATGAAGGGACAG |
| | | | | | PSLT042 | | | CGAAACCGACCGCTCAGATTCATTT |
| | | | | | | | | TATGGGATCGGTTGTTGAGGCAGG |
| | | | | | | | | CTGCTGGAATGACGTAGGAACCTTA |
| | | | | | | | | GAAATTCAATGCCATAATAAAGAGG |
| | | | | | | | | GAGTTGAACGTTATATTATTGTCGA |
| | | | | | | | | GAATATTATCACGCCGATATCGTCT |
| | | | | | | | | CCTCATGCAACGGTAAAACGAGATT |
| | | | | | | | | ATTTGGATGAAGATAAGCAATTAAC |
| | | | | | | | | AGTGCTACGCATTGTCTATGACTGA |
| | | | | | | | | ACCGCGTAGCAGACCGCAGATGGT |
| | | | | | | | | GTCCCGTCAGTGTCGTGTGAGAATA |
| | | | | | | | | TTA |
| 11.80 | 1.53 | 1.25 | 0.51 | 35187 | PSLT044 | | − | |
| 2.87 | 1.13 | 1.28 | 0.40 | 37474 | IR PSLT045- | | | CAATACGCTGGCCCAGCGGTTTGG |
| | | | | | PSLT046 | | | TGCTGTCATATTTAAACTGGACGGT |
| | | | | | | | | TTTAGATACGTGCAGCATACCGTTT |
| | | | | | | | | TTCAGATCGGCAGCGTGTGACATGA |
| | | | | | | | | TGGATTTCAGGTCCTTACCGCTGAT |
| | | | | | | | | TTCCATGCTCATGACATCGTTGGTG |
| | | | | | | | | AACGGATACATACTCAGCACATCAC |
| | | | | | | | | CATAGGTGATATTACCTTTAGGCAA |
| | | | | | | | | TTCGGTACGGATGCCGCCAGCATTA |
| | | | | | | | | TAGAAGGAAGCGTCGGCGCCAGGA |
| | | | | | | | | ACGGTAGCCATCAGGGCATCGGTG |
| | | | | | | | | ATTAAGTTGCCGGTTGGCGCGGATT |
| | | | | | | | | CACC |
| 10.57 | 1.16 | 0.91 | 0.60 | 38107 | PSLT046 | | − | |
| 5.16 | 1.15 | 1.60 | 1.64 | 38398 | IR PSLT046- | | | CATTATCCAACAATACCGGGAATTG |
| | | | | | PSLT047 | | | CAATTTGCTGAGTTGTTTAACCAGA |
| | | | | | | | | TTCTCATGGCCATGGTCAAATTCAT |
| | | | | | | | | GGTTACCGACAGAGACGGCGTCGT |
| | | | | | | | | AAGGCATGGTATTTAAAATATCAATA |
| | | | | | | | | ATAGCCTCGCCTTTGGTCAGCGTAC |
| | | | | | | | | TGATAAAAGGTCCGGTGAAATAGTC |
| | | | | | | | | GCCAGCATCAAAGAAAAAGACATCT |
| | | | | | | | | TTCTCTTTCGCTTTTGCATCTTTGAC |
| | | | | | | | | AATTTTCGAGATGGGCGCAAAGCC |
| | | | | | | | | GCCTACCGGACGTGTCTTGGATACA |
| | | | | | | | | TAGGGGATAATTTCTGGGGTTACATG |

Sequencing of Promoters.

One hundred and ninety-two clones from a library that underwent two rounds of enrichment in tumor (library-3) were picked at random and sequenced, yielding 100 different sequences. These were mapped to the genome and their potential regulation (tumor-specific activation, or activation in both spleen and tumor) was determined by comparison with the microarray data (see Table 5, presented below). The clones included 26 that were preferentially activated in tumors, and 40 that were activated both in tumor and spleen. 77% of the tumor enriched clones (20 of 26) and 75% of the clones induced in both tumor and spleen (30 of 40) mapped at least partly to intergenic regions. As expected, none of these 100 clones were spleen-specific. The 20 intergenic clones supported by both biological replicates on array experiments are presented in Tables 6A and 6B.

TABLE 5
Microarry status of active promoter clones in Salmonella

| | Promoter Status | | |
|---|---|---|---|
| | | | Preferentially |
| | | Active in Spleen | Active in |
| Genome Location | Not Detected | and Tumor | Tumor |
| Intragenic sequences | 27 | 10 | 6 |
| Intergenic sequences | 7 | 30 | 20 |

TABLE 6A
Cloned candidate intergenic tumor-specific Salmonella promoters

| | | Median ratio of experiment versus input | | | | |
|---|---|---|---|---|---|---|
| | Genome | | | Tumor | Tumor | Tumor |
| | position of | Clone | Spleen | (+) | (+)(−)(+) | (+)(−)(+) |
| Intergenic regions | peak signal | ID | Lib-1 | Lib-2 | Lib-3 | Lib-4 |
| STM0468-STM0469 | 526177 | 85 | 0.9 | 2.3 | 5.5 | 9.5 |
| STM0474-STM0475 | 529126 | 86 | 1.9 | 1.7 | 3.2 | 2.6 |
| STM0580-STM0581 | 638735 | 87 | 0.9 | 3.2 | 0.3 | 8.5 |
| STM0844-STM0845 | 914762 | 10 | 0.8 | 1.9 | 5.8 | 0.4 |
| STM0937-STM0938 | 1014704 | 11 | 0.7 | 4.2 | 6.5 | 10.3 |
| STM1382-STM1383 | 1466034 | 16 | 0.7 | 4.6 | 7.4 | 13.9 |
| STM1529-STM1530 | 1606103 | 20 | 1.9 | 5.5 | 2.8 | 13 |
| STM1807-STM1808 | 1909051 | 26 | 1.2 | 1.6 | 6.5 | 9.7 |
| STM1914-STM1915 | 2011503 | 28 | 0.9 | 3.9 | 7.2 | 7.5 |
| STM1996-STM1997 | 2079476 | 30 | 1.2 | 2.9 | 7.4 | 4 |
| STM2035-STM2036 | 2114187 | 31 | 1.3 | 5.9 | 4.7 | 8 |
| STM2261-STM2262 | 2359663 | 34 | 0.6 | 2.1 | 3.5 | 4.8 |
| STM2309-STM2310 | 2417301 | 36 | 0.6 | 2.7 | 6.5 | 6.3 |
| STM3070-STM3071 | 3233025 | 44 | 0.8 | 1.4 | 2.8 | 3.1 |
| STM3106-STM3107 | 3266543 | 45 | 1.1 | 3.5 | 4.6 | 4.6 |
| STM3525-STM3526 | 3688646 | 55 | 0.8 | 3.8 | 1.8 | 5.6 |
| STM3880-STM3881 | 4091492 | 61 | 0.9 | 5.4 | 0.1 | 13.8 |
| STM4289-STM4290 | 4530650 | 71 | 0.9 | 2 | 8.3 | 10 |
| STM4418-STM4419 | 4661108 | 77 | 0.8 | 3.4 | 8.3 | 6 |
| STM4430-STM4431 | 4674477 | 78 | 1.3 | 6.1 | 5.6 | 8 |

TABLE 6B
Cloned candidate intergenic tumor-specific Salmonella promoters

| | | | | 5′ | | 3′ | | Stable/ |
|---|---|---|---|---|---|---|---|---|
| Intergenic | Clone | Cloned | | gene | | gene | Anerobic | Unstable |
| regions | ID | Promoter | 5′ gene | orient | 3′ gene | orient | induction? | GFP |
| STM0468- | 85 | + | ylaB | − | rpmE2 | + | | Unstable |
| STM0469 | | | | | | | | |
| STM0474- | 86 | − | ybaJ | − | acrB | − | | Stable |
| STM0475 | | | | | | | | |
| STM0580- | 87 | − | STM0580 | − | STM0581 | + | | Stable |
| STM0581 | | | | | | | | |
| STM0844- | 10 | − | pflE | − | moeB | − | Yes | Unstable |
| STM0845 | | | | | | | | |
| STM0937- | 11 | − | hcp | − | ybjE | − | Yes | Unstable |
| STM0938 | | | | | | | | |
| STM1382- | 16 | − | orf408 | − | ttrA | − | | Stable |
| STM1383 | | | | | | | | |
| STM1529- | 20 | − | STM1529 | + | STM1530 | + | | Stable |
| STM1530 | | | | | | | | |
| STM1807- | 26 | + | dsbB | + | STM1808 | + | | Stable |
| STM1808 | | | | | | | | |
| STM1914- | 28 | − | flhB | − | cheZ | − | | Unstable |
| STM1915 | | | | | | | | |
| STM1996- | 30 | − | cspB | − | umuC | − | | Stable |
| STM1997 | | | | | | | | |
| STM2035- | 31 | − | cbiA | − | pocR | − | | Stable |
| STM2036 | | | | | | | | |
| STM2261- | 34 | − | napF | − | eco | + | Yes | Stable |
| STM2262 | | | | | | | | |
| STM2309- | 36 | − | menD | − | menF | − | | Stable |
| STM2310 | | | | | | | | |
| STM3070- | 44 | − | epd | − | STM3071 | + | | Unstable |
| STM3071 | | | | | | | | |
| STM3106- | 45 | − | ansB | − | yggN | − | Yes | Stable |
| STM3107 | | | | | | | | |
| STM3525- | 55 | + | glpE | + | glpD | + | | Stable |
| STM3526 | | | | | | | | |
| STM3880- | 61 | + | kup | + | rbsD | + | | Stable |
| STM3881 | | | | | | | | |
| STM4289- | 71 | − | phnA | − | proP | + | | Unstable |
| STM4290 | | | | | | | | |
| STM4418- | 77 | + | STM4418 | − | STM4419 | + | | Stable |
| STM4419 | | | | | | | | |
| STM4430- | 78 | + | STM4430 | − | STM4431 | + | | Stable |
| STM4431 | | | | | | | | |

Some possible tumor promoters mapped inside annotated genes; 23% of the sequenced clones (6 of 26) and 18% of candidates identified by microarray (19 of 105; see Table 7, presented below). Some “promoters” may be artifacts that could arise from a variety of effects such as the inherent high copy number of the plasmid clone, or mutations that cause the copy number to increase or a new promoter to be generated. However, based on data from Escherichia coli, a close relative of Salmonella, intragenic regions might indeed contain promoters, based on evidence from transcription start sites, binding sites for RNA polymerase (Reppas et al, “The transition between transcriptional initiation and elongation in E. coli is highly variable and often rate limiting”, Mol. Cell 24:747-757, 2006, Grainger et al, “Studies of the distribution of Escherichia coli cAMP-receptor protein and RNA polymerase along the E. coli chromosome”, Proc. Natl. Acad. Sci. USA 102:17693-17698, 2005), and sigma factors (Wade et al, “Extensive functional overlap between sigma factors in Escherichia coli”, Nat. Struct. Mol. Biol. 13:806-814, 2006) as well as motif finders (Tutukina et al, “Intragenic promoter-like sites in the genome of Eschericia coli discovery and functional implication”, J. Bioinform. Comput. Biol. 5:549-560, 2007). Further work may provide confirmatory evidence of promoter activity in some cases.

Some weaker promoters may generate detectable GFP in the stable, but not the destabilized, GFP plasmid library. Fifty clones sequenced after FACS selection could be assigned to either the stabilized or destabilized library. Forty of these were of the stable GFP variety versus an expected 25 of each type if there had been no bias. Therefore, the destabilized library is, as expected, underrepresented following FACS.

TABLE 7
Intragenic regions that induce higher GFP expression in tumor than in spleen

| | | | | | |
|---|---|---|---|---|---|
| | Tumor | Tumor | Tumor | | |
| Spleen | (+) | (+)(−)(+) | (+)(−)(+) | Genome | |
| lib1 | lib2 | lib3 | lib4 | position | in- |

| | | | | | | |
|---|---|---|---|---|---|---|
| Clone | Median of | of | | | tragenic | |
| ID | experiment versus | peak | | Gene | seq. | Gene |
| Seq'd | input library | signal | Gene | symbol | orient. | orient |

| | | | | | | | | | | |
|---|---|---|---|---|---|---|---|---|---|---|
| 1 | 0.64 | 3.16 | 4.47 | 3.01 | 40,802 | STM0035 | STM0035 | − | + | CCCGCGCTATGGCGTGGT |
| | | | | | | | | | | GCATCCTACGGGGTGGAT |
| | | | | | | | | | | TCGTAATGGCCAACATATT |
| | | | | | | | | | | GGCCGCGCAGATAAGATG |
| | | | | | | | | | | AGCGGCGAGTTTGTGAGC |
| | | | | | | | | | | TCTGAAGTGGTGAACTGG |
| | | | | | | | | | | CTGGATAATAAGAAAGACG |
| | | | | | | | | | | ATAATCCGTTCTTCTTATAT |
| | | | | | | | | | | GTCGCCTTTACCGAAGTCC |
| | | | | | | | | | | ATAGCCCGCTGGCGTCGC |
| | | | | | | | | | | CGAAAAAATACCTTGATAT |
| | | | | | | | | | | GTATTCGCAGTACATGACC |
| | | | | | | | | | | GACTACCAGAAGCAGCAT |
| | | | | | | | | | | CCGGATCTGTTCTACGGC |
| | | | | | | | | | | GACTGGGCAGACAAACCG |
| | | | | | | | | | | TGGCGCGGCACCGGCGAA |
| | | | | | | | | | | TATTAC |
| 84 | 0.61 | 1.48 | 3.99 | 2.76 | 558,116 | STM0498 | ybaR | − | − | CAATAGCCGGTTGGCATTG |
| | | | | | | | | | | CTGACGACGGTAATGGAA |
| | | | | | | | | | | GACAGCGCCATTGCCGCG |
| | | | | | | | | | | CCTGCTACTACCGGGTTTA |
| | | | | | | | | | | ACAAGGTACCGGTAAACG |
| | | | | | | | | | | GCCACAGAATACCGGCGG |
| | | | | | | | | | | CCACCGGGATACCAATGC |
| | | | | | | | | | | TGTTGTAGATAAATGCGCC |
| | | | | | | | | | | AAGCAGGTTTTGTTTCATA |
| | | | | | | | | | | TTGCGCAACGTCGCGCGC |
| | | | | | | | | | | GAAATGGCCAGCGCATCC |
| | | | | | | | | | | GCCACGCCCATCAGACTAT |
| | | | | | | | | | | GGCGCATCAGCGTAATCG |
| | | | | | | | | | | CCGCGGTTTCAATCGCCA |
| | | | | | | | | | | CATCGCTGCCGCCGCCCA |
| | | | | | | | | | | TCGCGATACCGACGTCCG |
| | | | | | | | | | | CCTGCGCC |
| 7 | 0.68 | 6.89 | 4.77 | 10.76 | 743,461 | STM0683 | nagA | − | − | TAGTCGACATGCAGACCAT |
| | | | | | | | | | | CGGCGATAACGCCGCAAT |
| | | | | | | | | | | AAATATCCGCTTCGTCCAG |
| | | | | | | | | | | AACAGCGCCAGCAAGGCC |
| | | | | | | | | | | CGGCTCACGCCCTGTAAT |
| | | | | | | | | | | GTACGGCATCGCGTTAAAC |
| | | | | | | | | | | AGGTGAGTCGCAAAGGTA |
| | | | | | | | | | | ATCCCGGCGCGGAAGCCC |
| | | | | | | | | | | GCTTTCGCCTCTTTTAACG |
| | | | | | | | | | | TCGCGTTGGAGTGACCTG |
| | | | | | | | | | | CGGAAACCACAATGCCCG |
| | | | | | | | | | | CATTCGCCAGTTTAGCGAT |
| | | | | | | | | | | TACGTCAGCAGGCACCATT |
| | | | | | | | | | | TCCGGCGCGAGTGTGACT |
| | | | | | | | | | | TTGGTGATGACGTCGGCAT |
| | | | | | | | | | | TATCGCATAAGAAATCGAC |
| | | | | | | | | | | CAGCG |
| 15 | 0.73 | 6.11 | 0.24 | 14.71 | 1,418,744 | STM1338 | pheT | + | + | ATGAATCCGGCTCTGCATC |
| | | | | | | | | | | CGGGACAGTCTGCGGCGA |
| | | | | | | | | | | TTTATCTGAAAGATGAACG |
| | | | | | | | | | | TATTGGTTTTATTGGGGTT |
| | | | | | | | | | | GTTCACCCTGAACTGGAAC |
| | | | | | | | | | | GTAAACTGGATCTGAATGG |
| | | | | | | | | | | TCGTACGCTGGTGTTTGAA |
| | | | | | | | | | | CTGGAATGGAATAAGCTCG |
| | | | | | | | | | | CAGACCGTATCGTGCCGC |
| | | | | | | | | | | AGGCGCGGGAGATTTCAC |
| | | | | | | | | | | GCTTCCCGGCCAACCGTC |
| | | | | | | | | | | GCGATATTGCGGTTGTTGT |
| | | | | | | | | | | TGCAGAAAACGTTCCCGCA |
| | | | | | | | | | | GCGGATATTTTATCCGAAT |
| | | | | | | | | | | GTAAGAAAGTTGGCGTAAA |
| | | | | | | | | | | TCAGGTAGTTGGCGTAAACT |
| 17 | 0.83 | 3.46 | 3.23 | 5.23 | 1,504,175 | STM1426 | ribE | + | + | CGTGCATCTCATTCCGGAA |
| | | | | | | | | | | ACGTTGGAACGTACTACGC |
| | | | | | | | | | | TTGGCAGAAAAAAACTGGG |
| | | | | | | | | | | TGAGCGTGTGAATATCGAG |
| | | | | | | | | | | ATCGATCCGCAAACGCAG |
| | | | | | | | | | | GCGGTTGTCGATACCGTA |
| | | | | | | | | | | GAACGCGTACTGGCTGCG |
| | | | | | | | | | | CGAGAAAATGCGGTCAGA |
| | | | | | | | | | | AATCAGGCCGACATTGGCT |
| | | | | | | | | | | AACGGAAAATAAGATTCCC |
| | | | | | | | | | | CCGCATGAAATGCGGGGG |
| | | | | | | | | | | AGATGATTAGCGAGGAAC |
| | | | | | | | | | | GCGCAGTCCGTTTTCAACG |
| | | | | | | | | | | CCGCGCGTAAATACCACCT |
| | | | | | | | | | | GCCAAAGCTGGATATCAC |
| | | | | | | | | | | GCGCGCGAAACGCACCCG |
| | | | | | | | | | | CGCAG |
| 56 | 0.70 | 6.90 | 4.49 | 23.58 | 3,523,313 | STM3355 | STM3355 | + | − | TTTCAACAGAGGTCGCTAC |
| | | | | | | | | | | GCCCACGCCAACCAGCAG |
| | | | | | | | | | | CGGCGGACAAGCGTTGAG |
| | | | | | | | | | | GCCGTAGCTGGTCATCAC |
| | | | | | | | | | | ATCCAGTACAAAGCGGGT |
| | | | | | | | | | | CACACCTTCATAGCCTGCA |
| | | | | | | | | | | CCCGGCATCAGCACCATC |
| | | | | | | | | | | GCTTTCCCCGGCAGAGAA |
| | | | | | | | | | | CAACCACCGCCCGCCATA |
| | | | | | | | | | | TAGGTATAAATGCTGCACT |
| | | | | | | | | | | GATCGGAATTGGGAACGA |
| | | | | | | | | | | TTTCCCAGAAGACCGTCG |
| | | | | | | | | | | GCGTACCTTTACCCACGTT |
| | | | | | | | | | | TTTACCGGTGTTGTATTCA |
| | | | | | | | | | | TCAAAAGTTTCTACGCTGT |
| | | | | | | | | | | TGTGGCGCAGCGGAGAAT |
| | | | | | | | | | | CTACAGT |

array data
only

| | | | | | | | |
|---|---|---|---|---|---|---|---|
| 0.91 | 7.43 | 3.70 | 5.41 | 18,084 | STM0018 | STM0018 | ACCCTGCAACAAGCGATG |
| | | | | | | | ATAAAATAAGCTATACTTC |
| | | | | | | | CGCTCGTGTTGCGAAACC |
| | | | | | | | GGTATACAATAAATATAAA |
| | | | | | | | AATTCCACGACTAAACCGA |
| | | | | | | | AGGTATTCGGTTATTACAC |
| | | | | | | | CGACTGGTCACAGTATGAC |
| | | | | | | | AGCCGTCTGCAAGGCAAT |
| | | | | | | | ATGTCCCAACCGGGCCGT |
| | | | | | | | GGTTATGATTTAACCAAAG |
| | | | | | | | TTTCACCGACGGCTTATGA |
| | | | | | | | CAAACTGATTTTTGGCTTT |
| | | | | | | | GTTGGCATCACCGGTTTCA |
| | | | | | | | GAAAAATTGATACAGAAGA |
| | | | | | | | CCGCGATGTCGTAGCAGA |
| | | | | | | | AGCGGCAGCGCTGTGCGG |
| | | | | | | | CAA |
| 0.92 | 2.12 | 4.85 | 6.29 | 1,071,228 | STM0984 | msbA | AAGAGGTACTGATTTTTGG |
| | | | | | | | CGGTCAGGAAGTCGAAAC |
| | | | | | | | TAAACGCTTTGATAAAGTC |
| | | | | | | | AGCAATAAGATGCGACTGC |
| | | | | | | | AAGGCATGAAAATGGTCTC |
| | | | | | | | TGCCTCGTCAATTTCCGAT |
| | | | | | | | CCTATCATTCAGCTCATTG |
| | | | | | | | CCTCGCTGGCGCTGGCGT |
| | | | | | | | TTGTCCTCTATGCTGCGAG |
| | | | | | | | CTTCCCAAGCGTAATGGAT |
| | | | | | | | AGCCTGACGGCAGGGACC |
| | | | | | | | ATCACCGTGGTGTTCTCCT |
| | | | | | | | CCATGATCGCGCTGATGC |
| | | | | | | | GTCCATTAAAATCGCTGAC |
| | | | | | | | AAACGTTAACGCGCAGTTC |
| | | | | | | | CAGCGTGGGATGGCGGCT |
| | | | | | | | TG |
| 0.46 | 3.08 | 2.56 | 4.03 | 1,342,729 | STM1258 | STM1258 | GCGCGAGACGCTGGTCGC |
| | | | | | | | CGTTATTACAGAATGTCTC |
| | | | | | | | TTTTGATATCGCGCCCGGC |
| | | | | | | | GAAATGGTGGCATTGGTTG |
| | | | | | | | GCGGCAGCGGGGAGGGC |
| | | | | | | | AAAAGTCTGCTGCTGCAAT |
| | | | | | | | GCCTGCTCGATCTGCTGC |
| | | | | | | | CGGAAAATTTACGCTTTCG |
| | | | | | | | GGGGGAGATTACGCTTGA |
| | | | | | | | TGGCAACCGGCTGGACAG |
| | | | | | | | ACATACCATCAGGCAGCTT |
| | | | | | | | AGGGGCAATACGTTTAGCT |
| | | | | | | | ACGTGCCGCAGGGGGTAC |
| | | | | | | | AGGCGCTTAATCCCATGCT |
| | | | | | | | GAATATCAGAAAACATTTG |
| | | | | | | | AACAGAGCATGTCATCTGA |
| | | | | | | | CCGG |
| 0.91 | 2.09 | 3.01 | 4.08 | 2,358,604 | STM2259 | napA | ATTGACCCGATCCAAACAT |
| | | | | | | | GCCGATCGCTTCTGGTCCT |
| | | | | | | | TTCTCTTTCAGGGAGGTTT |
| | | | | | | | TAAACTTCTCTTCCATCAC |
| | | | | | | | ATCGAAGGCCTGTTCCCA |
| | | | | | | | GCTCACCGGCGTAAACTC |
| | | | | | | | GCCGTCTTTGTGATAGCTG |
| | | | | | | | CCGTCTTTCATGCGCAGCA |
| | | | | | | | TCGGCTGCGTCAGACGAT |
| | | | | | | | CTTTACCGTACATGATTTT |
| | | | | | | | GGGCAGGAAGTAGCCTTT |
| | | | | | | | AATGCAGTTCAGACCACG |
| | | | | | | | GTTGACCGGCGCGTCGGG |
| | | | | | | | GTCGCCCTGGCAGGCGAC |
| | | | | | | | CACACGGCCCTGCTGCGT |
| | | | | | | | TCCCACCAACACACCGCAA |
| | | | | | | | CCCGT |
| 1.40 | 2.88 | 3.62 | 9.57 | 3,002,027 | STM2857 | hypD | CACATTACGCTGATCCCGA |
| | | | | | | | CGCTGCGTAGCCTACTGG |
| | | | | | | | AGCAGCCGGACAACGGCA |
| | | | | | | | TTGACGCCTTTCTTGCGCC |
| | | | | | | | AGGCCACGTCAGCATGGT |
| | | | | | | | CATCGGCACCGAGGCGTA |
| | | | | | | | CCAGTTTATCGCCGCCGAT |
| | | | | | | | TTTCATCGCCCGCTGGTG |
| | | | | | | | GTGGCTGGATTCGAACCG |
| | | | | | | | CTTGATCTACTGCAAGGCG |
| | | | | | | | TGGTCATGCTGGTTGAGCA |
| | | | | | | | GAAAATAGCGGCCCTAAG |
| | | | | | | | CCAGGTTGAAAATCAATAC |
| | | | | | | | CGTCGCGTGGTGCCGGAT |
| | | | | | | | GCCGGAAACATGCTGGCG |
| | | | | | | | CAGCAGGCCATTGCCGAT |
| | | | | | | | GTGTTCT |
| 0.74 | 2.66 | 7.94 | 22.93 | 3,026,126 | STM2882 | sipA | AGCAGCAGGGGTATCAAC |
| | | | | | | | GTTTGCATTTCAAGGTGCC |
| | | | | | | | GGGCTTCCCGTCCTACGC |
| | | | | | | | TGGTACCCTGCTCTTGCGT |
| | | | | | | | TAATTTTTGGTGGCACATA |
| | | | | | | | TCAAGCGCCTCAACAGCCT |
| | | | | | | | TCGCCGCCGCTTTGTCAAC |
| | | | | | | | AAGGTGCGTAAGATTGCTG |
| | | | | | | | CGGGTTAACGGATCTAAC |
| | | | | | | | GTACAGCCAAAGTTATGTT |
| | | | | | | | CAATGCAGCTGGCAATATA |
| | | | | | | | GGGCATCACCTCCTGCATA |
| | | | | | | | ACAAGATTCGTCGATAATT |
| | | | | | | | TACTTAATTCACCGCCAGT |
| | | | | | | | GTTATTTTTGATAATATCTA |
| | | | | | | | ACAGCTGCTTTCCAGGT |
| 0.74 | 3.02 | 5.85 | 17.96 | 3,087,704 | STM2945 | sopD | TAGAATCTATGAGTAGAGA |
| | | | | | | | GGAGAGACAATTATTTTTA |
| | | | | | | | CAAATATGTGAGGTGATTG |
| | | | | | | | GTTCGAAGATGACCTGGC |
| | | | | | | | ACCCGGAATTACTTCAGGA |
| | | | | | | | GTCGATTTCAACTCTACGA |
| | | | | | | | AAAGAAGTGACGGGAAAT |
| | | | | | | | GCACAAATCAAAACGGCG |
| | | | | | | | GTTTATGAGATGATGCGTC |
| | | | | | | | CCGCAGAGGCTCCAGACC |
| | | | | | | | ACCCGCTTGTCGAATGGC |
| | | | | | | | AGGACTCACTTACTGCAGA |
| | | | | | | | TGAAAAATCAATGCTGGCC |
| | | | | | | | TGTATTAATGCCGGTAACT |
| | | | | | | | TTGAGCCTACGACTCAGTT |
| | | | | | | | TTGCAAAATAGGTTATCAG |
| | | | | | | | GA |
| 0.81 | 3.08 | 3.19 | 7.02 | 3,472,959 | STM3304 | rplU | GTGAACCACTGACGATGG |
| | | | | | | | CCCTGCTGCTTACGGTAGT |
| | | | | | | | GTTTACGGCGACGAAACTT |
| | | | | | | | AACGATTTTAACTTTCTCG |
| | | | | | | | CCACGACCGTGGGCAACA |
| | | | | | | | ACTTCAGCTTTGATTACGC |
| | | | | | | | CGCCATCAACGAAAGGAA |
| | | | | | | | CGCCGATTTTGACTTCTTC |
| | | | | | | | ACCGTTTGCGATCATCAGA |
| | | | | | | | ACTTCAGCGAACTCGATAG |
| | | | | | | | TTTCGCCAGTTGCGATGTC |
| | | | | | | | CAGCTTTTCCAGGCGAAC |
| | | | | | | | GGTCTGACCTTCGCTTACT |
| | | | | | | | CGGTGTTGTTTACCACCAC |
| | | | | | | | TTTGGAAAACCGCGTACAT |
| | | | | | | | AAAAAACTCCGCTTCCGCGC |
| 0.73 | 2.63 | 2.53 | 5.18 | 3,660,088 | STM3502 | ompR | CGCCGGGCAGTTCGTTTG |
| | | | | | | | CCTGACGACGTAACACGG |
| | | | | | | | CGCGAATACGCGCCAACA |
| | | | | | | | GCTCGCGCGGGTTAAACG |
| | | | | | | | GTTTAGGAATGTAGTCATC |
| | | | | | | | GGCGCCGATTTCCAGCCC |
| | | | | | | | GACGATACGGTCAACCTCT |
| | | | | | | | TCACCCTTCGCCGTGACCA |
| | | | | | | | TAATGATCGGCATTGGATT |
| | | | | | | | ACTTTGACTACGCAGGCGA |
| | | | | | | | CGACAAATCGACAGACCAT |
| | | | | | | | CTTCACCTGGCAGCATTAA |
| | | | | | | | ATCCAGTACCATGAGATGG |
| | | | | | | | AAAGATTCACGGGTCAGCA |
| | | | | | | | GACGATCCATCTGCTCAGC |
| | | | | | | | GTTAGCGACGCTTCGAAC |
| | | | | | | | CTG |
| 0.89 | 3.00 | 3.86 | 3.92 | 3,957,871 | STM3758 | fidL | GCTTAATGCGTACAGAAAA |
| | | | | | | | ATATCGGGCGTTTCCCGAT |
| | | | | | | | GGTGAACATAAAGCCACG |
| | | | | | | | ATGGCCCTGAGTCAGGAT |
| | | | | | | | GGTGTAACTGATACTTTTC |
| | | | | | | | CCTGGATAGACATAAAAAT |
| | | | | | | | CGGGTAAAACCGTCTCGAT |
| | | | | | | | AACCGCATCGGACAGTGTT |
| | | | | | | | TCGTCACGCGTGACTTTGT |
| | | | | | | | TGATATCCGTCGATATAAA |
| | | | | | | | ATGGGTGCTGTCTTTATTT |
| | | | | | | | TCACTCCATACATAGGAAA |
| | | | | | | | CATCACGGCGGATCACGC |
| | | | | | | | CGCTCATTTTATTATCGAC |
| | | | | | | | GTAATATGTTCCGCTGATG |
| | | | | | | | GAAACCACCCCAGTGCGTT |
| 0.73 | 7.03 | 2.38 | 11.84 | 4,601,412 | STM4358 | amiB | CCGAACTGTTAGGCGGCG |
| | | | | | | | CTGGCGATGTGCTGGCGA |
| | | | | | | | ACAGTCAGTCAGACCCTTA |
| | | | | | | | CCTGAGCCAGGCGGTACT |
| | | | | | | | GGATTTGCAATTCGGTCAT |
| | | | | | | | TCGCAGCGGGTAGGGTAT |
| | | | | | | | GATGTGGCGACGAACGTA |
| | | | | | | | CTAAGCCAACTCGACGGC |
| | | | | | | | GTGGGGTCGCTGCATAAA |
| | | | | | | | CGCCGCCCGGAACACGCT |
| | | | | | | | AGCCTGGGCGTGTTGCGT |
| | | | | | | | TCGCCGGATATCCCGTCC |
| | | | | | | | ATTTTGGTGGAGACGGGC |
| | | | | | | | TTTATCAGTAATCACGGCG |
| | | | | | | | AAGAGCGATTGCTGGCGA |
| | | | | | | | GCGACCGCTATCAGCAGC |
| | | | | | | | AGATTGCTGA |
| 0.49 | 5.44 | 8.71 | 19.81 | 4,735,184 | STM4489 | STM4489 | TTTCCTGAATCAGACGTTT |
| | | | | | | | GAAAATACCGATAAACACA |
| | | | | | | | TCACGATAGTTTCTCCATG |
| | | | | | | | GCTAACCTGGCAAAAACTG |
| | | | | | | | GAGCAAACCGGTTTTCTTG |
| | | | | | | | ATTCCATGATTACGGCGTG |
| | | | | | | | TTCACGTGGTATTAACGTC |
| | | | | | | | ACGGTAGTCACTGACAGAA |
| | | | | | | | GCTACAACACTGAACATAA |
| | | | | | | | TGATTTTGAGAAGCGAAAA |
| | | | | | | | GAGAAGCAGCAGAACCTT |
| | | | | | | | AAAGCGGCGCTGGAGAAA |
| | | | | | | | CTGAACGCCCTTGGTATTG |
| | | | | | | | CGACAAAACTGGTCAATCG |
| | | | | | | | TGTTCATAGCAAAATTGTT |
| | | | | | | | ATTGGTGATGATGGTTTG |
| 0.64 | 11.20 | 6.44 | 19.39 | 4,748,275 | STM4496 | STM4496 | TTTGCGCGCCAGACGGGC |
| | | | | | | | AACCAGCAGCTTCACTTCT |
| | | | | | | | TCTTCCGGCCATCCATAAG |
| | | | | | | | GACGGCGGGCAAAGTGGT |
| | | | | | | | TCAGAATATCGCGTAAATA |
| | | | | | | | AACCGGCTTATTGAACTCG |
| | | | | | | | ATATTCATGCTGACCCAGG |
| | | | | | | | TTTCTACTTCGCGCATCGC |
| | | | | | | | GTCGGGGTTGGATTCCTC |
| | | | | | | | CAGTTCGCCCAGATCCAG |
| | | | | | | | CTCCGCATCATTCTCCACC |
| | | | | | | | GTGAGTAGTGCATGGATTT |
| | | | | | | | CACGTGCGATATCACCGTT |
| | | | | | | | GAACGGGCGCAGCATTTT |
| | | | | | | | CAGCTTGGCAAACGTGTTT |
| | | | | | | | TCAATCACATAGCGGCAAG |
| | | | | | | | CT |

Confirmation of Tumor Specificity of Individual Clones In Vivo.

Five cloned promoters potentially activated in bacteria growing in tumor but not in the spleen were selected to be individually confirmed in vivo. A group of tumor-bearing mice and normal mice were injected i.v. with bacteria containing the cloned promoters. Tumors and spleens were imaged after 2 days, at low and high resolution using the Olympus OV 100 small animal imaging system. Three of the five tumor-specific candidates (clones 10, 28, and 45) were induced much more in tumor than in spleen. Clone 44 produced low signals and clone 84 was highly expressed in tumor but was detectable in the spleen.

Among the most likely promoters to be uncovered in this study are those induced by hypoxia, which is thought to be an important contributor to Salmonella targeting of tumors (Mengesha et al, “Development of a flexible and potent hypoxia-inducible promoter for tumor-targeted gene expression in attenuated Salmonella”, Cancer Biol. Ther. 5:1120-1128, 2006). Salmonella promoters induced by hypoxia include those controlled directly or indirectly by the two global regulators of anaerobic metabolism, Fnr and ArcA (luchi and Weiner, Cellular and molecular physiology of Escherichia coli in the adaptation to aerobic environments”, J. Biochem. 120:1055-1063, 1996).

Clone 45 contains the promoter region of ansB, which encodes part of asparaginase. In E. coli, ansB is positively coregulated by Fnr and by CRP (cyclic AMP receptor protein), a carbon source utilization regulator (24). In S. enterica, the anaerobic regulation of ansB may require only CRP (Jennings et al, “Regulation of the ansB gene of Salmonella enterica”, Mol. Miicrobiol. 9:165-172, 1993, Scott et al, “Transcriptional co-activation at the ansB promoters: involvement of the activating regions of CRP and FNR when bound in tandem”, Mol. Microbiol. 18:521-531, 1995).

Clone 10 is the promoter region of a putative pyruvate-formate-lyase activating enzyme (pflE). This clone was only observed in library-3, but enrichment was considerable in that library (see Tables 2A and 2B). This clone was pursued further because the operon is co-regulated in E. coli by both ArcA and Fnr (Sawers and Suppmann, “Anaerobic induction of pyruvate formate-lyase gene expression is mediated by the ArcA and FNR proteins”, J. Bacteriol. 174:3474-3478, 1992, Knappe and Sawers, “A radical-chemical route to acetyl-CoA: the anaerobically induced pyruvate formate-lyase system of Escherichia coli”, FEMS Microbiol. Rev. 6:383-398, 1990).

Finally, clone 28 contains the promoter region of flhB, a gene that is required for the formation of the flagellar apparatus (Williams et al, “Mutations in fliK and flhB affecting flagellar hook and filament assembly in Salmonella typhimurium” J. Bacteriol. 178:2960-2970, 1996) and is not known to be regulated in anaerobic metabolism.

Further screening was performed on these three clones. Bacteria containing these clones were i.v. injected at 5×10⁶, 5×10⁷, and 5×10⁷ cfu into tumor and non-tumor-bearing nude mice. One or 2 days post-injection, spleens and tumors were imaged using the OV100 imaging system, homogenized, and the bacterial titer was quantified on LB+ Amp. Spleens from normal mice were compared with tumors that had a similar number of colony-forming units, so that any difference in fluorescence would be attributable to increased GFP expression rather than bacterial numbers. FIG. 2 confirms that tumors are much more fluorescent than spleens infected with the same number of bacteria for each of the three clones. A positive control that constitutively expresses TurboGFP resulted in strong fluorescence in spleen even with doses as low as 2×10⁵ cfu.

The Salmonella endogenous promoter for pepT is regulated by CRP and Fnr (Mengesha et al, 2006). In previous studies, the TATA and the Fnr binding sites of this promoter were modified to engineer a hypoxia-inducible promoter that drives reporter gene expression under both acute and chronic hypoxia in vitro (Mengesha et al, 2006). Induction of the engineered hypoxia-inducible promoter in vivo became detectable in mice 12 hours after death, when the mouse was globally hypoxic (Mengesha et al, 2006). In our experiments, the wild-type pepT intergenic region did not pass the threshold to be included in the tumor-specific promoter group. Perhaps the appropriate clone is not represented in the library, or induction (i.e., level of hypoxia in the PC3 tumors) was not enough for this particular promoter.

In summary, Salmonella thrives in the hypoxic conditions found in solid tumors (Mengesha et al, 2006). There are four promoters known to be regulated by hypoxia among the 20 sequenced intergenic clones (see Tables 2A and 2B), of which two (clones 10 and 45) were tested and shown to be induced in tumors (see FIG. 2). Many candidate promoters that seem to be preferentially activated within tumors may be unrelated to hypoxia, including clone 28 (FIG. 2). Any promoters that are later proven to respond in their natural context in the genome may illuminate conditions within tumors, other than hypoxia, that are sensed by Salmonella.

Attenuated Salmonella strains with tumor targeting ability can be used to deliver therapeutics under the control of promoters preferentially induced in tumors (Pawelek et al. “Tumor-targeted Salmonella as a novel anticancer vector”, Cancer Res 1997; 57:4537-44; Zhao et al. “Targeted therapy with a Salmonella typhimurium leucine-arginine auxotroph cures orthotopic human breast tumors in nude mice”, Cancer Res 2006; 66:7647-52; Zhao et al. “Tumor-targeting bacterial therapy with amino acid auxotrophs of GFP-expressing Salmonella typhimurium”, Proc Natl Acad Sci USA 2005; 102:755-60; Zhao et al. “Monotherapy with a tumor-targeting mutant of Salmonella typhimurium cures orthotopic metastatic mouse models of human prostate cancer”, Proc Natl Acad Sci USA 2007; Nishikawa et al. “In vivo antigen delivery by a Salmonella typhimurium type III secretion system for therapeutic cancer vaccines”, J Clin Invest 2006; 116:1946-54; Panthel et al. “Prophylactic anti-tumor immunity against a murine fibrosarcoma triggered by the Salmonella type III secretion system”, Microbes Infect 2006; 8:2539-46; Thamm et al. “Systemic administration of an attenuated, tumor-targeting Salmonella typhimurium to dogs with spontaneous neoplasia: phase I evaluation”, Clin Cancer Res 2005; 11:4827-34; Forbes et al. “Sparse initial entrapment of systemically injected Salmonella typhimurium leads to heterogeneous accumulation within tumors”, Cancer Res 2003; 63:5188-93; Toso et al. “Phase I study of the intravenous administration of attenuated Salmonella typhimurium to patients with metastatic melanoma”, J Clin Oncol 2002; 20:142-52; Avogadri, et al. “Cancer immunotherapy based on killing of Salmonella-infected tumor cells”, Cancer Res 2005; 65:3920-7). Such promoters are technically useful whether or not they are regulated in the same way in their natural context in the genome. These promoters would be tools to reduce the expression of the therapeutic in bacteria outside the tumor and thus reduce side-effects, and thereby produce a highly selective and effective therapy of metastatic cancer. Further sophistications are also possible. For example, combinations of two or more promoters that are preferentially induced in tumors by differing regulatory mechanisms would allow delivery of two or more separate protein components of a therapeutic system under different regulatory pathways. In addition, new promoter systems induced by external agents such as arabinose (Loessner et al. “Remote control of tumor-targeted Salmonella enterica serovar Typhimurium by the use of L-arabinose as inducer of bacterial gene expression in vivo”, Cell Microbiol. 9:1529-37, 2007) or salicylic acid (Royo et al. “In vivo gene regulation in Salmonella spp. by a salicylate-dependent control circuit”, Nat. Methods 4:937-42, 2007) allow promoters in Salmonella to be induced throughout the body at a time of choice. Such inducible regulation could be combined with tumor-specific Salmonella promoters to express useful products in the tumor only when the exogenous activator is added; therapy delivery would be exquisitely controlled both in time and space.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, yet these modifications and improvements are within the scope and spirit of the invention.

The invention illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the invention claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present invention has been specifically disclosed by representative embodiments and optional features, modification and variation of the concepts herein disclosed may be resorted to by those skilled in the art, and such modifications and variations are considered within the scope of this invention.

Certain embodiments of the invention are set forth in the claims that follow:

1. An isolated nucleic acid molecule which comprises a recombinant expression system, which expression system comprises a nucleotide sequence encoding a toxic or therapeutic RNA or protein, or an RNA or protein that participates in generating a toxin or therapeutic agent, operably linked to a heterologous promoter, which promoter is preferentially activated in solid tumors.

2. The isolated nucleic acid molecule of claim 1 wherein the promoter is an Enterobacteriaceae promoter.

3. The isolated nucleic acid molecule of claim 2 wherein the promoter is a Salmonella promoter.

4. The isolated nucleic acid molecule of claim 3, wherein the promoter comprises (i) a nucleotide sequence of Table 7A and Table 7B, or (ii) a functional promoter subsequence of (i).

5. (canceled)

6. Recombinant host cells that contain the nucleic acid molecule of claim 1.

7. The cells of claim 6 that are avirulent Salmonella.

8-9. (canceled)

10. A method for identifying a promoter preferentially activated in tumor tissue which method comprises:
(a) providing a library of expression systems each comprising a nucleotide sequence encoding a detectable protein operably linked to a different candidate promoter;
(b) providing said library to solid tumor tissue and to normal tissue;
(c) identifying cells from each tissue that show high levels of expression of the detectable protein; and
(d) obtaining said expression systems from the cells that produce greater levels of detectable protein in tumor tissue as compared to normal tissue, and identifying the promoters of said expression system.

11-15. (canceled)

16. The method of claim 10, which comprises scoring promoters identified in (d).

17-21. (canceled)

22. An expression system which comprises a first promoter nucleotide sequence operably linked to a first coding sequence and second promoter nucleotide sequence operably linked to a second coding sequence, wherein:
the first coding sequence and the second coding sequence encode polypeptides that individually do not inhibit tumor growth;
polypeptides encoded by the first coding sequence and the second coding sequence, in combination, inhibit tumor growth; and
the first promoter nucleotide sequence and the second promoter nucleotide sequence are preferentially activated in solid tumors.

23. The expression system of claim 22, wherein the first promoter nucleotide sequence and the second promoter nucleotide sequence are in the same nucleic acid molecule.

24. The expression system of claim 22, wherein the first promoter nucleotide sequence and the second promoter nucleotide sequence are in different nucleic acid molecules.

25. (canceled)

26. The expression system of claim 22, wherein the first promoter nucleotide sequence and the second promoter nucleotide sequence are Enterobacteriaceae sequences.

27. The expression system of claim 26, wherein the Enterobacteriaceae sequences are Salmonella sequences.

28. The expression system of claim 22, wherein:
the first coding sequence encodes an enzyme,
the second coding sequence encodes a prodrug, and
the enzyme processes the prodrug into a drug that inhibits tumor growth.

29. (canceled)

30. The expression system of claim 22, wherein the first promoter nucleotide sequence, the second promoter nucleotide sequence, or the first promoter nucleotide sequence and the second promoter nucleotide sequence comprise (i) a nucleotide sequence of Table 7A and Table 7B, (ii) a functional promoter nucleotide sequence 80% or more identical to a nucleotide sequence of Table 7A and Table 7B, or (iii) or a functional promoter subsequence of (i) or (ii).

31. (canceled)

32. Recombinant host cells that contain the expression system of claim 22.

33. The cells of claim 32 that are avirulent Salmonella.

34. An expression system which comprises three or more heterologous promoter nucleotide sequences operably linked to three or more coding sequences, wherein the promoter nucleotide sequences are preferentially activated in solid tumors.

35-44. (canceled)
